(19) United States
(12) Patent Application Publication
(10) Pub. No.: US 2026/0212953 A1
(43) Pub. Date: Jul. 23, 2026

(54) An Integrative Framework to Identify Therapeutic Molecules for Treating Preterm Birth

(71) Applicant: The Regents of the University of California, Oakland, CA (US)

(72) Inventors: Jingjing Li, San Francisco, CA (US); Cheng Wang, San Francisco, CA (US)

(21) Appl. No.: 19/142,914

(22) PCT Filed: Jan. 12, 2024
(86) PCT No.: PCT/US2024/011372

§ 371 Date: Jun. 24, 2025

Related U.S. Application Data
(60) Provisional application No. 63/440,317, filed on Jan. 20, 2023.

(51) Int. Cl. G16B 20/20; G16B 15/10; G16B 30/00; G16B 40/20; G16B 50/00; G16H 10/40; G16H 10/60; G16H 20/10; G16H 50/30

(52) U.S. Cl. CPC G16B 20/20; G16B 15/10; G16B 30/00; G16B 40/20; G16B 50/00; G16H 10/40; G16H 10/60; G16H 20/10; G16H 50/30

(57) ABSTRACT

Methods, systems, and devices, including computer programs encoded on a computer storage medium are provided for genome-wide identification of non-coding somatic mutations associated with preterm birth. A predictive deep learning model is provided that estimates the risk of preterm birth for an individual based on detection of non-coding somatic mutations that alter tissue-specific chromatin structure resulting in gene regulatory changes that lead to myometrial transition to preterm labor. Methods of predicting therapeutic responsiveness of an individual to treatment with progestin for preterm labor and methods of treating preterm labor are also provided.

### CROSS-REFERENCE TO RELATED APPLICATION

This application claims benefit under 35 U.S.C. § 119 (e) of provisional application 63/440,317, filed Jan. 20, 2023, which application is hereby incorporated by reference in its entirety.

### BACKGROUND

Preterm birth (delivery prior to 37 weeks of gestation) is the leading cause of neonatal mortality and morbidity that annually affects 15 million pregnancies worldwide (Goldenberg et al. (2008) Lancet 371, 75-84; Lee et al. (2019) Lancet Glob Health 7, e2-e3; Romero et al. (2014) Science 345, 760-765). Compared with medically indicated cases (e.g. pre-eclampsia), many preterm birth cases are spontaneous (sPTB) (Chen et al. (2021) Front Neurol 12, 649749; Gyamfi-Bannerman and Ananth (2014) Obstet Gynecol 124, 1069-1074; Loftin et al. (2010) Rev Obstet Gynecol 3, 10-19) resulting from a premature conversion of the myometrium (the thick smooth-muscle layer of the uterus) from a quiescent to a contractile state. Myometrial quiescence is regulated by the progesterone receptor (PR, isoforms PR-A and PR-B), which blocks labor by suppressing contractile proteins and pro-labor inflammatory factors throughout pregnancy (Amini et al. (2019) Mol Cell Endocrinol 479, 1-11; Nadeem et al. (2017) Sci Rep 7, 13357). For example, during pregnancy, progesterone/PR signaling blocks myometrial cell NF-kB activation to achieve its anti-inflammatory function (Hardy et al., 2006), and further suppresses expression of genes encoding proteins involved in myometrial contractility (Lindstrom and Bennett (2005) Reproduction 130, 569-581) (e.g. the oxytocin receptor (Fuchs et al. (1984) Am J Obstet Gynecol 150, 734-741), cyclooxygenase-2 (Soloff et al. (2004) Endocrinology 145, 1248-1254), and the prostaglandin F₂α receptor (Olson (2003) Best Pract Res Clin Obstet Gynaecol 17, 717-730). Transition of the myometrium from quiescence to labor is initiated by functional progesterone withdrawal, where the anti-inflammatory progesterone-PR signaling is attenuated due to PR isoform alterations (Merlino et al., 2007; Nadeem et al., 2016). This change subsequently promotes pro-labor inflammatory stimuli to induce tissue-level inflammation that induces myometrial contraction and the initiation of parturition (Stanfield et al. (2019) Front Genet 10, 185; Tan et al. (2012) J Clin Endocrinol Metab 97, E719-730). Given the critical role of PGR in regulating labor timing, our recent work together with previous studies has associated the presence of genomic variants in PGR with preterm birth risk (Ehn et al. (2007) Nucleic Acids Res 46, D649-D655; Li et al. (2018) Am J Hum Genet 103, 45-57; Manuck et al. (2010) Obstet Gynecol 115, 765-770). As such, in clinical practice, progestin (i.e., compounds that mimic progesterone actions to maintain pregnancy) therapy (e.g., hydroxyprogesterone caproate injection) has been developed for preventing preterm labor. However, this approach has been associated with heterogeneous clinical outcomes (Blackwell et al. (2020) Am J Perinatol 37, 127-136; Group (2021) Lancet 397, 1183-1194; Meis et al. (2003) N Engl J Med 348, 2379-2385, likely due to substantial genetic heterogeneity and the multi-factorial nature underlying preterm labor. Therefore, identifying the complete genetic architecture of myometrial contractility at parturition and myometrial cell progesterone/PR signaling would be critical for the development of pre-screening assays to personalize and improve the efficacy of preterm birth prevention therapy.

### SUMMARY

Methods, systems, and devices, including computer programs encoded on a computer storage medium are provided for genome-wide identification of non-coding somatic mutations associated with preterm birth. A predictive deep learning model is provided that estimates the risk of preterm birth for an individual based on detection of non-coding somatic mutations that alter tissue-specific chromatin structure resulting in gene regulatory changes that lead to myometrial transition to preterm labor. Methods of predicting therapeutic responsiveness of an individual to treatment with progestin for preterm labor and methods of treating preterm labor are also provided.

In one aspect, a method for genome-wide identification of non-coding somatic mutations associated with preterm birth is provided, the method comprising: a) providing a database comprising epigenomic correlation data for associations between non-coding somatic mutations and chromatin structural changes associated with myometrial transition to preterm labor based on genome-wide epigenomic screening of a population of patients experiencing preterm birth; b) generating a deep learning model to compute the probability that a given genomic sequence has an open chromatin structure; and c) using the deep learning model to identify non-coding somatic mutations associated with the myometrial transition to preterm labor, wherein a non-coding somatic mutation is considered to contribute to risk of preterm birth if an allelic change from its corresponding reference wild-type allele to the somatic mutation results in an alteration in predicted chromatin openness based on the deep learning model.

In certain embodiments, the deep learning model uses a deep residual neural network or deep convolutional neural network.

In certain embodiments, the method further comprises calculating deep estimation from epigenome prediction plus (DEEP+) scores for each non-coding somatic mutation that is identified as contributing to the risk of preterm birth.

In certain embodiments, the DEEP+ scores are used in combination with genome-wide association study (GWAS) risk scores for each non-coding somatic mutation to determine the risk of preterm birth.

In certain embodiments, the DEEP+ scores are used in combination with haploinsufficiency scores for each non-coding somatic mutation to determine the risk of preterm birth.

In certain embodiments, the DEEP+ scores are used in combination with myometrial transcriptomic profiling data to determine the risk of preterm birth.

In certain embodiments, the method further comprises using a Bayesian estimation for altered regulation (BEAR) model to calculate a BEAR composite risk score for each non-coding somatic mutation that is identified as contributing to the risk of preterm birth, wherein the BEAR model uses a mixture Gaussian model of the distribution of the DEEP+ scores, the GWAS risk scores, the gene haploinsufficiency scores, and the myometrial transcriptomic profiling data to calculate the BEAR composite risk score, wherein the BEAR composite risk score is used to determine the risk of preterm birth for an individual.

In certain embodiments, the patient is European or African American.

In certain embodiments, one or more of the non-coding somatic mutations are in genes that regulate myometrial muscle relaxation or inflammatory responses.

In certain embodiments, the non-coding somatic mutations are in an intronic genomic region, a promoter, a 5′ untranslated region (5′ UTR), a 3′ untranslated region (3′ UTR), an exonic genomic region, an intergenic genomic region, or a genomic region encoding a non-coding RNA.

In certain embodiments, the non-coding somatic mutations comprise at least one insertion, deletion, or single-nucleotide variant.

In certain embodiments, the epigenomic correlation data comprises assay for transposase-accessible chromatin sequencing (ATAC-Seq) data.

In another aspect, a method of predicting risk of preterm birth for an individual is provided, the method comprising: a) obtaining a biological sample from the individual; b) genotyping one or more cells in the biological sample to determine if the individual has one or more non-coding somatic mutations associated with the risk of preterm birth; and c) calculating a composite DEEP+ score for the one or more non-coding somatic mutations associated with the risk of preterm birth detected by genotyping, wherein the composite DEEP+ score indicates the risk of preterm birth.

In certain embodiments, the composite DEEP+ score is used in combination with genome-wide association study (GWAS) risk scores for each non-coding somatic mutation associated with the risk of preterm birth detected by genotyping to determine the risk of preterm birth.

In certain embodiments, the composite DEEP+ score is used in combination with haploinsufficiency scores for each non-coding somatic mutation associated with the risk of preterm birth detected by genotyping to determine the risk of preterm birth.

In certain embodiments, the composite DEEP+ score is used in combination with myometrial transcriptomic profiling data to determine the risk of preterm birth.

In certain embodiments, the method further comprises using a Bayesian estimation for altered regulation (BEAR) model to calculate a composite BEAR risk score for each non-coding somatic mutation associated with the risk of preterm birth detected by genotyping, wherein the BEAR model uses a mixture Gaussian model of the distribution of the DEEP+ scores, the GWAS risk scores, the gene haploinsufficiency scores, and the myometrial transcriptomic profiling data to calculate the composite BEAR risk score, wherein the composite BEAR risk score indicates the risk of preterm birth.

In certain embodiments, the non-coding somatic mutations are in an intronic genomic region, a promoter, a 5′ untranslated region (5′ UTR), a 3′ untranslated region (3′ UTR), an exonic genomic region, an intergenic genomic region, or a genomic region encoding a non-coding RNA.

In certain embodiments, the non-coding somatic mutations comprise at least one insertion, deletion, or single-nucleotide variant.

In certain embodiments, the one or more non-coding somatic mutations associated with the risk of preterm birth comprise one or more non-coding somatic mutations selected from Table 1.

In certain embodiments, genotyping comprises sequencing at least part of a genome of a cell from the biological sample.

In certain embodiments, genotyping comprises sequencing the whole genome of a cell from the biological sample.

In certain embodiments, the biological sample is a myometrium sample.

In certain embodiments, the method further comprises treating the individual to reduce the risk of preterm birth if the composite DEEP+ score indicates the individual is at risk of preterm birth.

In certain embodiments, the method further comprises treating the individual to reduce the risk of preterm birth if the composite BEAR risk score indicates the individual is at risk of preterm birth.

In certain embodiments, the method further comprises administering progestin to the individual if the DEEP+ score or BEAR risk score indicates the individual is at risk of preterm birth.

In certain embodiments, the method further comprises predicting non-responsiveness of the individual to treatment with progestin based on identifying one or more non-coding somatic mutations in one or more genes selected from the group consisting of AHNAK, ANTXR2, ATP1B1, ATP2B4, CALM2, CAPZA2, CAV1, CDC42EP3, CITED2, CNN1, CORO1C, CPQ, CSDE1, DCN, DPP6, DPYSL3, DST, DSTN, DYNC1LI2, FHL1, FILIP1L, GSN, HADH, HSPB8, IGFBP7, ITM2B, KANK2, KCNMA1, LDB2, MAP4, MBNL1, MFAP5, MGP, MSRB3, MYH11, MYLK, MYO1C, NR2F2, PALLD, PARVA, PBX1, PGR, PKD2, PLN, PLS3, PPP1R12B, PRUNE2, PTN, RAP2C, RSPO3, SERINC1, SH3BGRL, SLMAP, SORBS1, SPARCL1, SUN1, SVIL, SYNPO2, TACC1, TBC1D1, TCEAL4, TES, TIMP2, TJP1, TMEM123, TNS1, TPM1, YAP1, and YWHAZ.

In another aspect, a database comprising DEEP+ scores for a plurality of non-coding somatic mutations associated with preterm birth is provided.

In certain embodiments, the database comprises or consists of DEEP+ scores for non-coding somatic mutations selected from Table 1.

In certain embodiments, the database further comprises BEAR risk scores for the plurality of non-coding somatic mutations associated with preterm birth.

In another aspect, a computer implemented method for predicting risk of preterm birth for an individual is provided, the computer performing steps comprising: a) receiving genome sequencing data for an individual; b) identifying non-coding somatic mutations associated with preterm birth present in the individual from the genome sequencing data, wherein the individual has a plurality of non-coding somatic mutations selected from Table 1; c) calculating a composite deep estimation from epigenome prediction plus (DEEP+) risk score for the non-coding somatic mutations detected in the individual by genotyping using a database described herein, wherein the composite DEEP+ score indicates the risk of preterm birth for the individual; and d) displaying information regarding the risk of preterm birth for the individual.

In certain embodiments, the computer implemented method further comprises calculating a composite BEAR risk score for the non-coding somatic mutations detected in the individual by genotyping using a database described herein, wherein the composite BEAR risk score indicates the risk of preterm birth for the individual.

In certain embodiments, the computer implemented method further comprises storing the information regarding the risk of preterm birth for the individual in a database.

In another aspect, a system for predicting the risk of preterm birth for an individual using a computer implemented method, described herein, is provided, the system comprising: a) a storage component for storing data, wherein the storage component has instructions for predicting the risk of preterm birth for an individual based on analysis of the genome sequencing data stored therein; b) a computer processor for processing the genome sequencing data using one or more algorithms, wherein the computer processor is coupled to the storage component and configured to execute the instructions stored in the storage component in order to receive the inputted genome sequencing data and analyze the data according to the computer implemented method described herein; and c) a display component for displaying the information regarding the risk of preterm birth for the individual.

In another aspect, a non-transitory computer-readable medium is provided, the non-transitory computer-readable medium comprising program instructions that, when executed by a processor in a computer, causes the processor to perform the computer implemented method for predicting risk of preterm birth for an individual, as described herein.

In another aspect, a kit comprising the non-transitory computer-readable medium described herein and instructions for predicting the risk of preterm birth for an individual are provided.

In another aspect, a method of predicting therapeutic responsiveness of an individual to treatment with progestin for preterm labor is provided, the method comprising: a) obtaining a biological sample from the individual; b) genotyping one or more cells in the biological sample to determine if the individual has one or more non-coding somatic mutations associated with risk of preterm birth in one or more genes selected from the group consisting of AHNAK, ANTXR2, ATP1B1, ATP2B4, CALM2, CAPZA2, CAV1, CDC42EP3, CITED2, CNN1, CORO1C, CPQ, CSDE1, DCN, DPP6, DPYSL3, DST, DSTN, DYNC1LI2, FHL1, FILIP1L, GSN, HADH, HSPB8, IGFBP7, ITM2B, KANK2, KCNMA1, LDB2, MAP4, MBNL1, MFAP5, MGP, MSRB3, MYH11, MYLK, MYO1C, NR2F2, PALLD, PARVA, PBX1, PGR, PKD2, PLN, PLS3, PPP1R12B, PRUNE2, PTN, RAP2C, RSPO3, SERINC1, SH3BGRL, SLMAP, SORBS1, SPARCL1, SUN1, SVIL, SYNPO2, TACC1, TBC1D1, TCEAL4, TES, TIMP2, TJP1, TMEM123, TNS1, TPM1, YAP1, and YWHAZ; and c) calculating a composite DEEP+ score for the one or more non-coding somatic mutations detected in the individual by genotyping using the database described herein, wherein if the composite DEEP+ score is above a reference threshold value, the individual is identified as a non-responder who will not benefit from the treatment with the progestin, and wherein if the composite DEEP+ score is below a reference threshold value, the individual is identified as a responder who will benefit from the treatment with the progestin.

In certain embodiments, the method further comprises calculating a composite BEAR risk score for the non-coding somatic mutations detected in the individual by genotyping using a database described herein, wherein if the composite BEAR risk score is above a reference threshold value, the individual is identified as a non-responder who will not benefit from the treatment with the progestin, and wherein if the composite BEAR risk score is below a reference threshold value, the individual is identified as a responder who will benefit from the treatment with the progestin.

In certain embodiments, the method further comprises administering progestin to the individual if the individual is identified as a responder.

In certain embodiments, the genotyping comprises sequencing at least part of a genome of a cell from the biological sample.

In certain embodiments, the genotyping comprises sequencing the whole genome of a cell from the biological sample.

In certain embodiments, the biological sample is a myometrium sample.

In another aspect, a computer implemented method for predicting therapeutic responsiveness of an individual to treatment with progestin for preterm labor is provided, the computer performing steps comprising: a) receiving genome sequencing data for an individual; b) identifying one or more non-coding somatic mutations associated with risk of preterm birth in one or more genes selected from the group consisting of AHNAK, ANTXR2, ATP1B1, ATP2B4, CALM2, CAPZA2, CAV1, CDC42EP3, CITED2, CNN1, CORO1C, CPQ, CSDE1, DCN, DPP6, DPYSL3, DST, DSTN, DYNC1LI2, FHL1, FILIP1L, GSN, HADH, HSPB8, IGFBP7, ITM2B, KANK2, KCNMA1, LDB2, MAP4, MBNL1, MFAP5, MGP, MSRB3, MYH11, MYLK, MYO1C, NR2F2, PALLD, PARVA, PBX1, PGR, PKD2, PLN, PLS3, PPP1R12B, PRUNE2, PTN, RAP2C, RSPO3, SERINC1, SH3BGRL, SLMAP, SORBS1, SPARCL1, SUN1, SVIL, SYNPO2, TACC1, TBC1D1, TCEAL4, TES, TIMP2, TJP1, TMEM123, TNS1, TPM1, YAP1, and YWHAZ; c) calculating a composite DEEP+ score for the one or more non-coding somatic mutations detected in the individual by genotyping using a database described herein, wherein if the composite DEEP+ score is above a reference threshold value, the individual is identified as a non-responder who will not benefit from the treatment with the progestin, and wherein if the composite DEEP+ score is below a reference threshold value, the individual is identified as a responder who will benefit from the treatment with the progestin; and d) displaying information regarding whether the individual is identified as a non-responder or a responder.

In certain embodiments, the computer implemented method further comprises calculating a BEAR risk score for the non-coding somatic mutations detected in the individual by genotyping using a database described herein, wherein if the composite BEAR risk score is above a reference threshold value, the individual is identified as a non-responder who will not benefit from the treatment with the progestin, and wherein if the composite BEAR risk score is below a reference threshold value, the individual is identified as a responder who will benefit from the treatment with the progestin.

In certain embodiments, the computer implemented method further comprises storing the information regarding whether the individual is identified as a non-responder or a responder in a database.

In another aspect, a system for predicting therapeutic responsiveness of an individual to treatment with progestin for preterm labor using the computer implemented method, described herein, is provided, the system comprising: a) a storage component for storing data, wherein the storage component has instructions for predicting the therapeutic responsiveness of an individual to treatment with progestin based on analysis of the genome sequencing data stored therein; b) a computer processor for processing the genome sequencing data using one or more algorithms, wherein the computer processor is coupled to the storage component and configured to execute the instructions stored in the storage component in order to receive the inputted genome sequencing data and analyze the data according to the computer implemented method for predicting the therapeutic responsiveness of an individual to treatment with progestin, described herein; and c) a display component for displaying the information regarding whether the individual is identified as a responder or a non-responder.

In another aspect, a non-transitory computer-readable medium comprising program instructions that, when executed by a processor in a computer, causes the processor to perform a computer implemented method for predicting the therapeutic responsiveness of an individual to treatment with progestin, described herein, is provided.

In another aspect, a kit comprising the non-transitory computer-readable medium described herein and instructions for predicting the therapeutic responsiveness of an individual to treatment with progestin is provided.

In another aspect, a method of treating preterm labor in a pregnant female subject is provided, the method comprising administering a therapeutically effective amount of a composition comprising RKI-1447, bisindolylmaleimide-ix, TBBt, LY294002, URMC099, Bosutinib, or SB-203580 to the pregnant female subject.

In certain embodiments, the composition is administered orally, intravenously, intramuscularly, or vaginally.

In certain embodiments, the composition is administered locally to the myometrium.

In certain embodiments, the pregnant female subject is having preterm labor or identified as having a risk of preterm labor.

In certain embodiments, the multiple cycles of treatment are administered to the pregnant female subject. In some embodiments, the composition is administered daily or intermittently. In some embodiments, the composition is administered to the pregnant female subject during pregnancy beginning at 16 to 20 weeks of gestation. In some embodiments, the composition is administered to the pregnant female subject until delivery.

In another aspect, a composition comprising RKI-1447, bisindolylmaleimide-ix, TBBt, LY294002, URMC099, Bosutinib, or SB-203580 for use in a method of treating preterm labor is provided. In some embodiments, the composition further comprises a pharmaceutically acceptable excipient.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1M. A machine learning framework for identifying genomic loci in spontaneous preterm birth. (FIG. 1A) The flowchart of the deep learning model for quantifying mutational consequences on altering local chromatin architecture in the term pregnant myometrium (non-laboring). (FIG. 1B) The enhanced performance based on the DEEP+ model compared with the original DEEP model. (FIGS. 1C, 1D) Validation of the DEEP+ model performance on independently collected clinical samples. Performance was evaluated by the areas under the receiver operating characteristic curve (AUROC, FIG. 1C) and the precision-recall curve (AUPR, FIG. 1D). (FIG. 1E) High-confidence eQTLs (HC_eQTLs) in the uterus received the highest DEEP+ score, followed by low-confidence eQTLs (LC_eQTLs) and non-eQTL loci in the uterus. (FIG. 1F) The hierarchical Bayesian model to compute a posterior probability of each genomic locus for its implication in spontaneous preterm labor conditioned on its mutational effect (the DEEP+ score), disease risk estimated from GWAS Z scores (maternal genomes), dosage sensitivity and molecular activity of the affected gene(s). (FIG. 1G) A graphical representation of the Bayesian model, where nodes represent variables and edges denote dependencies among stochastic variables. The model integrates mutational deleteriousness scores (the DEEP+ score, X), GWAS effect sizes (the Z scores), gene haploinsufficiency scores (H), and gene expression (E) in a tissue of interest to derive a posterior probability of any genomic locus for its disease association. F={Z, H, E}. The graph was then used for variational inference. See Supplementary Information for details of model construction and variational learning. (FIG. 1H) An example of the model output, where genomic mutations in SORBS1 were highly scored by the model for their implication in spontaneous preterm labor, evidenced by the ATAC-seq signals showing strong regulatory activity, by the DEEP+ scores showing mutational deleteriousness in altering local chromatin architecture, and by the GWAS Z scores showing the increased risk for developing spontaneous preterm labor. SORBS1 also received a high haploinsufficiency score (pLI scores) indicating its dosage sensitivity and its expression in the term pregnancy myometrium (non-laboring) was also confirmed. FIG. 1 (I) The LD (linkage disequilibrium) linked heterozygous loci in the SORBS1 gene body with the identified promoter loci displayed significant allele-specific expression relative to all heterozygous loci in the term pregnant myometrial transcriptome. P values were derived from Wilcoxon rank-sum test. (FIG. 1J) Variant-level model checking confirmed increased mutational effects (DEEP+ scores) and risk for spontaneous preterm labor (GWAS Z scores) for the identified genomic loci. (FIG. 1K) Gene-level model checking confirmed increased gene expression in the non-laboring term pregnant myometrium and gene dosage sensitivity (haploinsufficiency scores) for the identified genomic loci. TP stands for term pregnant. (FIG. 1L) Performing the analysis using an independent African American cohort with spontaneous preterm birth revealed a shared genetic architecture with individuals with European ancestries. (FIG. 1M) The enriched TF binding motifs among the flanking sequences centered on the identified 1,079 identified BEAR loci.

FIGS. 2A-2B. Functional analysis of the identified genomic loci in spontaneous preterm birth. (FIG. 2A) Gene ontology analysis revealed the enriched functional categories, particularly including the muscle contraction/relaxation module and the immune response module. (FIG. 2B) Genes affected by the identified genomic loci were highly enriched for target genes bound by the progesterone receptor in the non-laboring term pregnancy myometrium. P values were derived from Fisher's exact test.

FIGS. 3A-3L Mechanistic investigation of the identified genomic loci in spontaneous preterm birth. (FIG. 3A) The identified genes displayed up-regulation in the term-pregnancy myometrium (nonlaboring) relative to the non-pregnant myometrium. NP, TP stand for non-pregnant and term pregnant, respectively. P values were derived from Wilcoxon rank-sum test. (FIG. 3B) The identified genes displayed significant down-regulation upon labor onset. TIL and TNL stand for term in labor and term not in labor, respectively. (FIG. 3C) 99 genes displayed differential gene expression upon labor onset, where genes in group 1 (G1) and group 2 (G2) exhibited down- and up-regulation in primary myometrium samples, respectively, at the labor onset. (FIG. 3D) Validation of G1 and G2 gene expression dynamics during labor on an independent myometrium transcriptome dataset from 5 TNL and 5 TIL samples. (FIGS. 3E-3G) Gene expression variation of the identified genes (G1 and G2) is correlated with uterine contractility (FIG. 3E), cervical dilation diameter (FIG. 3F) and neonatal birth weight (FIG. 3G). Gene expression variation was represented by the first principal components (PC1) computed from expression values of the G1 and G2 genes among 31 individuals with varying physiological status. The p and P values were derived from Spearman correlation test. (FIGS. 3H-31) The enriched functional categories for G1 (panel H) and G2 (panel I) genes, respectively. (FIG. 3J) Only G1 genes displayed significant up-regulation in primary myometrial cell cultures when treated with forskolin (FSK) to promote myometrial relaxation. The identified genes (G1 and G2) were not responsive to the IL-1B treatment. P values were derived from Wilcoxon rank-sum test. (FIG. 3K) Both G1 and G2 genes were enriched for genes transcriptionally targeted by the progesterone receptor in the non-laboring term pregnant myometrium. P values were derived from Fisher's exact test. (FIG. 3L) The schematic model describing mechanistic modes of the progesterone receptor (PR) in pregnant myometrium before (left) and during (right) labor onset. PR interacts with different sets of co-factors to maintain uterine quiescence before labor onset and induce parturition before labor onset. MRP, muscle relaxation protein; CAP, contractility associated protein; P4, progesterone; PR-B, the isoform B of PR; PR-A, the isoform A of PR; PPI, protein-protein interaction.

FIGS. 4A-4D. Genetic contribution to the outcomes of progestin treatment for recurrent preterm labor. (FIGS. 4A-4B) The non-responders to the treatment tended to have increased mutation load ablating the myometrial chromatin architecture in the G1 genes (FIG. 4A), but not in the G2 genes (FIG. 4B), relative to the responders. NR and R stand for non-responders and responders, respectively. P values were derived from Wilcoxon rank-sum test. (FIGS. 4C-4D) The load of the deleterious regulatory mutations in the G1 genes predicted the responses to progestin treatment for recurrent preterm labor. Performance was evaluated by the areas under the receiver operating characteristic curve (AUROC, FIG. 4C) and the precision-recall curve (AUPR, FIG. 4D). The optimal threshold was indicated on the curves corresponding to 0 false positive rate (~100% specificity) and ~50% true positive rate (sensitivity).

FIGS. 5A-5H. Identifying small molecules for treating spontaneous preterm labor. (FIG. 5A) The algorithm ranks all the 4,293 FDR-approved drugs, clinical trial drugs, and pre-clinical tool compounds based on the confidence on their treatment effectiveness on spontaneous preterm labor. (FIG. 5B) The top 50 highly ranked drug displayed significant functional associations with the preterm birth genes identified in this study, where the bottom ranked 50 drugs exhibited the least functional associations with the preterm birth genes from this study. (FIG. 5C) Our model predicted scores and rank percentiles for drugs that have been used or are under clinical trials to treat spontaneous preterm birth. (FIG. 5D) The top ten candidate drugs predicted by our model for experimental validation. (FIG. 5E) The effects on increasing or decrease myometrial cell contractility determined by the collagen gel contraction assay when treating primary human uterine smooth muscle cells (HUtSMC) with the identified small molecules. The treatment effectiveness was determined in the contractile (stimulated by OXY, oxytocin) or in the quiescence state (PBS). P values were derived from Wilcoxon rank-sum test. (FIG. 5F) The dose-response curve for the top candidate drug RKI-1447 in quiescent HUtSMCs. The inset shows the dose-response curve of HUtSMCs treated with different concentrations of RKI-1447. (FIG. 5G) Studying the effects of RKI-1447 on the multi-scale network identified its functional associations with many muscle proteins with the strongest association with MYLK. (FIG. 5H) Docking analysis between RKI-1447 and MYLK confirmed their binding affinity.

FIGS. 6A-6D. Mutational burden analysis on 48 individuals with recurrent sPTB. (FIG. 6A) Population ancestry analysis identified a subgroup of 36 individuals with African ancestry. (FIGS. 6B-6C) Increased load of deleterious regulatory mutations in G1 genes was confirmed on all 48 individuals (FIG. 6B), and the lack of signal in G2 genes was also confirmed (FIG. 6C). (FIG. 6D) The predictability of clinical outcomes was confirmed on all 48 recruited females in this clinical trial. All p values were derived from Wilcoxon rank-sum test.

### DETAILED DESCRIPTION OF EMBODIMENTS

Methods, systems, and devices, including computer programs encoded on a computer storage medium are provided for genome-wide identification of non-coding somatic mutations associated with preterm birth. A predictive deep learning model is provided that estimates the risk of preterm birth for an individual based on detection of non-coding somatic mutations that alter tissue-specific chromatin structure resulting in gene regulatory changes that lead to myometrial transition to preterm labor. Methods of predicting therapeutic responsiveness of an individual to treatment with progestin for preterm labor and methods of treating preterm labor are also provided.

Before the present methods, systems, and devices are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the nucleic acid” includes reference to one or more nucleic acids and equivalents thereof, e.g., polynucleotides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Biological sample. The term “sample” with respect to an individual encompasses blood, urine, and other liquid samples of biological origin, solid tissue samples such as a biopsy or specimen or tissue cultures or cells derived or isolated therefrom and the progeny thereof. The definition also includes samples that have been manipulated in any way after their procurement, such as by treatment with reagents; washed; or enrichment for certain cell populations, such as myometrium cells. The definition also includes samples that have been enriched for particular types of molecules, e.g., nucleic acids, polypeptides, etc.

DNA samples, e.g., samples useful in genotyping, are readily obtained from any nucleated cells of an individual, e.g. hair follicles, cheek swabs, white blood cells, cells from myometrium tissue, etc., as known in the art.

The term “biological sample” encompasses a clinical sample. The types of “biological samples” include, but are not limited to: biological fluids, tissue samples, tissue obtained by surgical resection, tissue obtained by biopsy, cells in culture, cell supernatants, cell lysates, organs, bone marrow, blood, plasma, serum, saliva, urine, fine needle aspirate, lymph node aspirate, cystic aspirate, a paracentesis sample, a thoracentesis sample, and the like.

Obtaining and assaying a sample. The term “assaying” is used herein to include the physical steps of manipulating a biological sample to generate data related to the sample. As will be readily understood by one of ordinary skill in the art, a biological sample must be “obtained” prior to assaying or genotyping cells in the sample. Thus, the term “assaying” or “genotyping” implies that the sample has been obtained. The terms “obtained” or “obtaining” as used herein encompass the act of receiving an extracted or isolated biological sample. For example, a testing facility can “obtain” a biological sample in the mail (or via delivery, etc.) prior to assaying the sample. In some such cases, the biological sample was “extracted” or “isolated” from an individual by another party prior to mailing (i.e., delivery, transfer, etc.), and then “obtained” by the testing facility upon arrival of the sample. Thus, a testing facility can obtain the sample and then assay the sample, thereby producing data related to the sample.

The terms “obtained” or “obtaining” as used herein can also include the physical extraction or isolation of a biological sample from a subject. Accordingly, a biological sample can be isolated from a subject (and thus “obtained”) by the same person or same entity that subsequently assays or genotypes cells in the sample. When a biological sample is “extracted” or “isolated” from a first party or entity and then transferred (e.g., delivered, mailed, etc.) to a second party, the sample was “obtained” by the first party (and also “isolated” by the first party), and then subsequently “obtained” (but not “isolated”) by the second party. Accordingly, in some embodiments, the step of obtaining does not comprise the step of isolating a biological sample.

In some embodiments, the step of obtaining comprises the step of isolating a biological sample (e.g., a pre-treatment biological sample, a post-treatment biological sample, etc.). Methods and protocols for isolating various biological samples (e.g., a blood sample, a urine sample, a biopsy sample, a surgical specimen, an aspirate, etc.) will be known to one of ordinary skill in the art and any convenient method may be used to isolate a biological sample.

The terms “determining”, “measuring”, “evaluating”, “assessing,” “assaying,” and “analyzing” are used interchangeably herein to refer to any form of measurement, and include determining if an element is present or not.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those having preterm labor, etc.) as well as those in which prevention is desired (e.g., those at risk of preterm labor, etc.).

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

“Substantially purified” generally refers to isolation of a substance (e.g., compound, molecule, agent) such that the substance comprises the majority percent of the sample in which it resides. Typically, in a sample, a substantially purified component comprises 50%, preferably 80%-85%, more preferably 90-95% of the sample.

By “isolated” is meant an indicated cell, population of cells, or molecule is separate and discrete from a whole organism or is present in the substantial absence of other cells or biological macromolecules of the same type.

The terms “subject,” “individual” or “patient” are used interchangeably herein and refer to a vertebrate, preferably a mammal. By “vertebrate” is meant any member of the subphylum Chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered.

As used herein, the term “probe” refers to a polynucleotide that contains a nucleic acid sequence complementary to a nucleic acid sequence present in the target nucleic acid analyte (e.g., at location of a somatic mutation). The polynucleotide regions of probes may be composed of DNA, and/or RNA, and/or synthetic nucleotide analogs. Probes may be labeled in order to detect the target sequence. Such a label may be present at the 5′ end, at the 3′ end, at both the 5′ and 3′ ends, and/or internally.

An “allele-specific probe” hybridizes to only one of the possible alleles of a gene (e.g., hybridizes at the location of a mutation) under suitably stringent hybridization conditions.

The term “primer” as used herein, refers to an oligonucleotide that hybridizes to the template strand of a nucleic acid and initiates synthesis of a nucleic acid strand complementary to the template strand when placed under conditions in which synthesis of a primer extension product is induced, i.e., in the presence of nucleotides and a polymerization-inducing agent such as a DNA or RNA polymerase and at suitable temperature, pH, metal concentration, and salt concentration. The primer is preferably single-stranded for maximum efficiency in amplification, but may alternatively be double-stranded. If double-stranded, the primer can first be treated to separate its strands before being used to prepare extension products. This denaturation step is typically effected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA or RNA synthesis. Typically, nucleic acids are amplified using at least one set of oligonucleotide primers comprising at least one forward primer and at least one reverse primer capable of hybridizing to regions of a nucleic acid flanking the portion of the nucleic acid to be amplified.

An “allele-specific primer” matches the sequence exactly of only one of the possible alleles of a gene (e.g., hybridizes at the location of a mutation), and amplifies only one specific allele if it is present in a nucleic acid amplification reaction.

The term “common genetic variant” or “common variant” refers to a genetic variant having a minor allele frequency (MAF) of greater than 5%.

The term “rare genetic variant” or “rare variant” refers to a genetic variant having a minor allele frequency (MAF) of less than or equal to 5%.

### Methods

Methods are provided for genome-wide identification of deleterious non-coding somatic mutations associated with preterm birth. A predictive deep learning model is provided that estimates the risk of preterm birth for an individual based on detection of non-coding somatic mutations that alter tissue-specific chromatin structure resulting in gene regulatory changes that lead to myometrial transition to preterm labor. Methods are also provided for predicting therapeutic responsiveness of an individual to treatment with progestin.

The methods typically involve tissue-specific genotyping of an individual to identify deleterious non-coding somatic mutations present in the genome of cells and calculating a composite DEEP+ score for the non-coding somatic mutations detected by genotyping, wherein the composite DEEP score indicates whether the individual is at risk of preterm birth. Cells of interest for genotyping and analysis according to the subject methods include myometrial cells.

A deep learning model is used to evaluate the effect of each somatic mutation on chromatin openness compared to a reference allele. A somatic allele is considered deleterious if an allelic change from a reference allele (e.g., in the cellular genome of normal healthy tissue of the individual) to the somatic allele results in an alteration of the predicted chromatin status. For each somatic mutation, a DEEP score is used to quantify the overall allelic impact on chromatin openness. The chromatin status of a given genomic region can be predicted using the deep learning model based on the sequence of somatic alleles in the region by calculating a composite DEEP score for the somatic alleles.

Additionally, a database is provided comprising DEEP+ scores for a plurality of non-coding somatic mutations associated with preterm birth, wherein the DEEP+ scores are calculated using the predictive deep learning model as described further below (e.g., see Examples). In certain embodiments, the database comprises or consists of DEEP+ scores for non-coding somatic mutations selected from Table 1.

The methods described herein are useful for identifying individuals in need of close monitoring and treatment for preterm labor. Individuals at high risk of preterm birth may be monitored more frequently for preterm labor.

In addition, the methods described herein may be useful for determining that an individual should be administered a therapy to inhibit preterm labor and prevent preterm birth. In certain embodiments, a therapy is administered to a patient if an individual is identified as being at risk of preterm birth by the methods described herein. Treatment may include administering progestin, RKI-1447, bisindolylmaleimide-ix, TBBt, LY294002, URMC099, Bosutinib, or SB-203580, or a combination thereof, to the individual.

### Genotyping

Individuals may be genotyped to detect non-coding somatic mutations by any convenient method known in the art. Non-coding somatic mutations associated with preterm birth may include common or rare genetic variants, such as mutations (e.g., nucleotide replacements, insertions, or deletions) in an intronic genomic region, a promoter, a 5′ untranslated region (5′ UTR), a 3′ untranslated region (3′ UTR), an exonic genomic region, an intergenic genomic region, or a genomic region encoding a non-coding RNA. In certain embodiments, the non-coding somatic mutations are single nucleotide variants. In some embodiments, the non-coding somatic mutations are in dosage-sensitive genes.

For genetic testing, a biological sample containing nucleic acids is collected from an individual. The biological sample can be any sample from bodily fluids, tissue or cells that contains genomic DNA or RNA of the individual. In some embodiments, the biological sample is myometrium tissue or cells. In certain embodiments, nucleic acids from the biological sample are isolated, purified, and/or amplified prior to analysis using methods well-known in the art. See, e.g., Green and Sambrook Molecular Cloning: A Laboratory Manual (Cold Spring Harbor Laboratory Press; 4th edition, 2012); and Current Protocols in Molecular Biology (Ausubel ed., John Wiley & Sons, 1995); herein incorporated by reference in their entireties.

Detection of a mutation can be direct or indirect. For example, the mutated DNA itself can be detected directly. Alternatively, the mutation can be detected indirectly from cDNAs, amplified RNAs or DNAs, or proteins expressed by a mutated allele. Any method that detects a base change in a nucleic acid sample or an amino acid change in a protein can be used. For example, allele-specific probes that specifically hybridize to a nucleic acid containing the mutated sequence can be used to detect the mutation. A variety of nucleic acid hybridization formats are known to those skilled in the art. For example, common formats include sandwich assays and competition or displacement assays. Hybridization techniques are generally described in Hames, and Higgins “Nucleic Acid Hybridization, A Practical Approach,” IRL Press (1985); Gall and Pardue, Proc. Natl. Acad. Sci. U.S.A., 63:378-383 (1969); and John et al Nature, 223:582-587 (1969).

Sandwich assays are commercially useful hybridization assays for detecting or isolating nucleic acids. Such assays utilize a “capture” nucleic acid covalently immobilized to a solid support and a labeled “signal” nucleic acid in solution. The clinical sample will provide the target nucleic acid. The “capture” nucleic acid and “signal” nucleic acid probe hybridize with the target nucleic acid to form a “sandwich” hybridization complex.

In one embodiment, the allele-specific probe is a molecular beacon. Molecular beacons are hairpin shaped oligonucleotides with an internally quenched fluorophore. Molecular beacons typically comprise four parts: a loop of about 18-30 nucleotides, which is complementary to the target nucleic acid sequence; a stem formed by two oligonucleotide regions that are complementary to each other, each about 5 to 7 nucleotide residues in length, on either side of the loop; a fluorophore covalently attached to the 5′ end of the molecular beacon, and a quencher covalently attached to the 3′ end of the molecular beacon. When the beacon is in its closed hairpin conformation, the quencher resides in proximity to the fluorophore, which results in quenching of the fluorescent emission from the fluorophore. In the presence of a target nucleic acid having a region that is complementary to the strand in the molecular beacon loop, hybridization occurs resulting in the formation of a duplex between the target nucleic acid and the molecular beacon. Hybridization disrupts intramolecular interactions in the stem of the molecular beacon and causes the fluorophore and the quencher of the molecular beacon to separate resulting in a fluorescent signal from the fluorophore that indicates the presence of the target nucleic acid sequence.

For detection, the molecular beacon is designed to only emit fluorescence when bound to a specific allele of a gene. When the molecular beacon probe encounters a target sequence with as little as one non-complementary nucleotide, the molecular beacon preferentially stay in its natural hairpin state and no fluorescence is observed because the fluorophore remains quenched. See, e.g., Nguyen et al. (2011) Chemistry 17(46):13052-13058; Sato et al. (2011) Chemistry 17(41):11650-11656; Li et al. (2011) Biosens Bioelectron. 26(5):2317-2322; Guo et al. (2012) Anal. Bioanal. Chem. 402(10):3115-3125; Wang et al. (2009) Angew. Chem. Int. Ed. Engl. 48(5):856-870; and Li et al. (2008) Biochem. Biophys. Res. Commun. 373(4):457-461; herein incorporated by reference in their entireties.

In another embodiment, detection of the mutated sequence is performed using allele-specific amplification. In the case of PCR, amplification primers can be designed to bind to a portion of one of the disclosed genes, and the terminal base at the 3′ end is used to discriminate between the major and minor alleles or mutant and wild-type forms of the genes. If the terminal base matches the major or minor allele, polymerase-dependent three prime extension can proceed. Amplification products can be detected with specific probes. This method for detecting point mutations or polymorphisms is described in detail by Sommer et al. in Mayo Clin. Proc. 64:1361-1372 (1989).

Tetra-primer ARMS-PCR uses two pairs of primers that can amplify two alleles of a gene in one PCR reaction. Allele-specific primers are used that hybridize at the location of the mutated sequence, but each matches perfectly to only one of the possible alleles. If a given allele is present in the PCR reaction, the primer pair specific to that allele will amplify that allele, but not the other allele of the gene. The two primer pairs for the different alleles may be designed such that their PCR products are of significantly different length, which allows them to be distinguished readily by gel electrophoresis. See, e.g., Muñoz et al. (2009) J. Microbiol. Methods. 78(2):245-246 and Chiapparino et al. (2004) Genome. 47(2):414-420; herein incorporated by reference.

Mutations in a gene may also be detected by ligase chain reaction (LCR) or ligase detection reaction (LDR). The specificity of the ligation reaction is used to discriminate between the major and minor alleles of a gene. Two probes are hybridized at the site of the mutation in a nucleic acid of interest, whereby ligation can only occur if the probes are identical to the target sequence. See e.g., Psifidi et al. (2011) PLOS One 6(1):e14560; Asari et al. (2010) Mol. Cell. Probes. 24(6):381-386; Lowe et al. (2010) Anal Chem. 82(13):5810-5814; herein incorporated by reference.

As another example, an array comprising probes for detecting mutant alleles can be used. For example, SNP arrays are commercially available from Affymetrix and Illumina, which use multiple sets of short oligonucleotide probes for detecting known SNPs. The design of SNP arrays, such as manufactured by Affymetrix or Illumina, is described further in LaFamboise, “Single nucleotide polymorphism arrays: a decade of biological, computational and technological advances,” Nuc. Acids Res. 37(13):4181-4193 (2009).

Another method that can be used for detection of mutant alleles is PCR-dynamic allele specific hybridization (DASH), which involves dynamic heating and coincident monitoring of DNA denaturation, as disclosed by Howell et al. (Nat. Biotech. 17:87-88, 1999). A target sequence is amplified (e.g., by PCR) using one biotinylated primer. The biotinylated product strand is bound to a streptavidin-coated microtiter plate well (or other suitable surface), and the non-biotinylated strand is rinsed away with alkali wash solution. An oligonucleotide probe, specific for one allele (e.g., the wild-type allele), is hybridized to the target at low temperature. This probe forms a duplex DNA region that interacts with a double strand-specific intercalating dye. When subsequently excited, the dye emits fluorescence proportional to the amount of double-stranded DNA (probe-target duplex) present. The sample is then steadily heated while fluorescence is continually monitored. A rapid fall in fluorescence indicates the denaturing temperature of the probe-target duplex. Using this technique, a single-base mismatch between the probe and target results in a significant lowering of melting temperature (Tm) that can be readily detected.

A variety of other techniques can be used to detect mutations, including but not limited to, the Invader assay with Flap endonuclease (FEN), the Serial Invasive Signal Amplification Reaction (SISAR), the oligonucleotide ligase assay, restriction fragment length polymorphism (RFLP), single-strand conformation polymorphism, temperature gradient gel electrophoresis (TGGE), and denaturing high performance liquid chromatography (DHPLC). See, for example Molecular Analysis and Genome Discovery (R. Rapley and S. Harbron eds., Wiley 1^{st} edition, 2004); Jones et al. (2009) New Phytol. 183(4):935-966; Kwok et al. (2003) Curr. Issues Mol. Biol. 5(2):43-60; Muñoz et al. (2009) J. Microbiol. Methods. 78(2):245-246; Chiapparino et al. (2004) Genome. 47(2):414-420; Olivier (2005) Mutat. Res. 573(1-2):103-110; Hsu et al. (2001) Clin. Chem. 47(8):1373-1377; Hall et al. (2000) Proc. Natl. Acad. Sci. U.S.A. 97(15):8272-8277; Li et al. (2011) J. Nanosci. Nanotechnol. 11(2):994-1003; Tang et al. (2009) Hum. Mutat. 30(10):1460-1468; Chuang et al. (2008) Anticancer Res. 28(4A):2001-2007; Chang et al. (2006) BMC Genomics 7:30; Galeano et al. (2009) BMC Genomics 10:629; Larsen et al. (2001) Pharmacogenomics 2(4):387-399; Yu et al. (2006) Curr. Protoc. Hum. Genet. Chapter 7: Unit 7.10; Lilleberg (2003) Curr. Opin. Drug Discov. Devel. 6(2):237-252; and U.S. Pat. Nos. 4,666,828; 4,801,531; 5,110,920; 5,268,267; 5,387,506; 5,691,153; 5,698,339; 5,736,330; 5,834,200; 5,922,542; and 5,998,137 for a description of such methods; herein incorporated by reference in their entireties.

In certain embodiments, a probe set is used, wherein the probe set comprises a plurality of allele-specific probes for detecting deleterious non-coding somatic mutations in the subject's genome. The probe set may comprise one or more allele-specific polynucleotide probes. An allele-specific probe hybridizes to only one of the possible alleles of a gene under suitably stringent hybridization conditions. Individual polynucleotide probes comprise a nucleotide sequence derived from the nucleotide sequence of the target mutated allele sequences or complementary sequences thereof. The nucleotide sequence of the polynucleotide probe is designed such that it corresponds to, or is complementary to the target mutated allele sequences. The allele-specific polynucleotide probe can specifically hybridize under either stringent or lowered stringency hybridization conditions to a region of the target mutated allele sequences, to the complement thereof, or to a nucleic acid sequence (such as a cDNA) derived therefrom.

The selection of the allele-specific polynucleotide probe sequences and determination of their uniqueness may be carried out in silico using techniques known in the art, for example, based on a BLASTN search of the polynucleotide sequence in question against gene sequence databases, such as the Human Genome Sequence, UniGene, dbEST or the non-redundant database at NCBI. In one embodiment of the invention, the allele-specific polynucleotide probe is complementary to the region of a single mutated allele target DNA or mRNA sequence. Computer programs can also be employed to select allele-specific probe sequences that may not cross hybridize or may not hybridize non-specifically.

The allele-specific polynucleotide probes of the present invention may range in length from about 15 nucleotides to the full length of the coding target or non-coding target. In one embodiment of the invention, the polynucleotide probes are at least about 15 nucleotides in length. In another embodiment, the polynucleotide probes are at least about 20 nucleotides in length. In a further embodiment, the polynucleotide probes are at least about 25 nucleotides in length. In another embodiment, the polynucleotide probes are between about 15 nucleotides and about 500 nucleotides in length. In other embodiments, the polynucleotide probes are between about 15 nucleotides and about 450 nucleotides, about 15 nucleotides and about 400 nucleotides, about 15 nucleotides and about 350 nucleotides, about 15 nucleotides and about 300 nucleotides, about 15 nucleotides and about 250 nucleotides, about 15 nucleotides and about 200 nucleotides in length. In some embodiments, the probes are at least 15 nucleotides in length. In some embodiments, the probes are at least 15 nucleotides in length. In some embodiments, the probes are at least 20 nucleotides, at least 25 nucleotides, at least 50 nucleotides, at least 75 nucleotides, at least 100 nucleotides, at least 125 nucleotides, at least 150 nucleotides, at least 200 nucleotides, at least 225 nucleotides, at least 250 nucleotides, at least 275 nucleotides, at least 300 nucleotides, at least 325 nucleotides, at least 350 nucleotides, at least 375 nucleotides in length.

The allele-specific polynucleotide probes of a probe set can comprise RNA, DNA, RNA or DNA mimetics, or combinations thereof, and can be single-stranded or double-stranded. Thus, the polynucleotide probes can be composed of naturally-occurring nucleobases, sugars and covalent internucleoside (backbone) linkages as well as polynucleotide probes having non-naturally-occurring portions which function similarly. Such modified or substituted polynucleotide probes may provide desirable properties such as, for example, enhanced affinity for a target gene and increased stability. The probe set may comprise a coding target and/or a non-coding target. Preferably, the probe set comprises a combination of a coding target and non-coding target.

In another embodiment, a set of allele-specific primers is used, wherein the set of allele-specific primers comprises a plurality of allele-specific primers for detecting deleterious non-coding somatic mutations associated with preterm birth in the subject's genome. An allele-specific primer matches the sequence exactly of only one of the possible somatic alleles, hybridizes at the location of the deleterious non-coding somatic mutation, and amplifies only one specific mutated allele if it is present in a nucleic acid amplification reaction. For use in amplification reactions such as PCR, a pair of primers can be used for detection of a mutated allele sequence. Each primer is designed to hybridize selectively to a single allele at the site of the mutation in the gene under stringent conditions, particularly under conditions of high stringency, as known in the art. The pairs of allele-specific primers are usually chosen so as to generate an amplification product of at least about 50 nucleotides, more usually at least about 100 nucleotides. Algorithms for the selection of primer sequences are generally known, and are available in commercial software packages. These primers may be used in standard quantitative or qualitative PCR-based assays for SNP genotyping of subjects. Alternatively, these primers may be used in combination with probes, such as molecular beacons in amplifications using real-time PCR.

A label can optionally be attached to or incorporated into an allele-specific probe or primer polynucleotide to allow detection and/or quantitation of a target mutated allele sequence. The target mutated polynucleotide may be from genomic DNA, expressed RNA, a cDNA copy thereof, or an amplification product derived therefrom, and may be the positive or negative strand, so long as it can be specifically detected in the assay being used. Similarly, an antibody may be labeled that detects a polypeptide expression product of the mutated allele.

In certain multiplex formats, labels used for detecting different mutant alleles may be distinguishable. The label can be attached directly (e.g., via covalent linkage) or indirectly, e.g., via a bridging molecule or series of molecules (e.g., a molecule or complex that can bind to an assay component, or via members of a binding pair that can be incorporated into assay components, e.g. biotin-avidin or streptavidin). Many labels are commercially available in activated forms which can readily be used for such conjugation (for example through amine acylation), or labels may be attached through known or determinable conjugation schemes, many of which are known in the art.

Detectable labels useful in the practice of the invention may include any molecule or substance capable of detection, including, but not limited to, fluorescers, chemiluminescers, chromophores, bioluminescent proteins, enzymes, enzyme substrates, enzyme cofactors, enzyme inhibitors, isotopic labels, semiconductor nanoparticles, dyes, metal ions, metal sols, ligands (e.g., biotin, streptavidin or haptens) and the like. The term “fluorescer” refers to a substance or a portion thereof which is capable of exhibiting fluorescence in the detectable range. Particular examples of labels which may be used in the practice of the invention include, but are not limited to, SYBR green, SYBR gold, a CAL Fluor dye such as CAL Fluor Gold 540, CAL Fluor Orange 560, CAL Fluor Red 590, CAL Fluor Red 610, and CAL Fluor Red 635, a Quasar dye such as Quasar 570, Quasar 670, and Quasar 705, an Alexa Fluor such as Alexa Fluor 350, Alexa Fluor 488, Alexa Fluor 546, Alexa Fluor 555, Alexa Fluor 594, Alexa Fluor 647, and Alexa Fluor 784, a cyanine dye such as Cy 3, Cy3.5, Cy5, Cy5.5, and Cy7, fluorescein, 2′, 4′, 5′, 7′-tetrachloro-4-7-dichlorofluorescein (TET), carboxyfluorescein (FAM), 6-carboxy-4′,5′-dichloro-2′,7′-dimethoxyfluorescein (JOE), hexachlorofluorescein (HEX), rhodamine, carboxy-X-rhodamine (ROX), tetramethyl rhodamine (TAMRA), FITC, dansyl, umbelliferone, dimethyl acridinium ester (DMAE), Texas red, luminol, and quantum dots, enzymes such as alkaline phosphatase (AP), beta-lactamase, chloramphenicol acetyltransferase (CAT), adenosine deaminase (ADA), aminoglycoside phosphotransferase (neo^{r}, G418^{r}) dihydrofolate reductase (DHFR), hygromycin-B-phosphotransferase (HPH), thymidine kinase (TK), β-galactosidase (lacZ), and xanthine guanine phosphoribosyltransferase (XGPRT), beta-glucuronidase (gus), placental alkaline phosphatase (PLAP), and secreted embryonic alkaline phosphatase (SEAP). Enzyme tags are used with their cognate substrate. Detectable labels also include chemiluminescent labels such as luminol, isoluminol, acridinium esters, and peroxyoxalate and bioluminescent proteins such as firefly luciferase, bacterial luciferase, Renilla luciferase, and aequorin. Detectable labels also include isotopic labels, including radioactive and non-radioactive isotopes, such as, ³H, ²H, ¹²⁰I, ¹²³I, ¹²⁴I, ¹²⁵I, ¹³¹I, ³⁵S, ¹¹C, ¹³C, ¹⁴C, ³²P, ¹⁵N, ¹³N, ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹⁵⁴Gd, ¹⁵⁵Gd, ¹⁵⁶Gd, ¹⁵⁷Gd, ¹⁵⁸Gd, ¹⁵O, ¹⁸⁶Re, ¹⁸⁸Re, ⁵¹M, ^{52m}Mn, ⁵⁵Co, ⁷²As, ⁷⁵Br, ⁷⁶Br, ^{82m}Rb, and ⁸³Sr. Detectable labels also include color-coded microspheres of known fluorescent light intensities (see e.g., microspheres with xMAP technology produced by Luminex (Austin, TX); microspheres containing quantum dot nanocrystals, for example, containing different ratios and combinations of quantum dot colors (e.g., Qdot nanocrystals produced by Life Technologies (Carlsbad, CA); glass coated metal nanoparticles (see e.g., SERS nanotags produced by Nanoplex Technologies, Inc. (Mountain View, CA); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), glass microparticles with digital holographic code images (see e.g., CyVera microbeads produced by Illumina (San Diego, CA), near infrared (NIR) probes, and nanoshells. Detectable labels also include contrast agents such as ultrasound contrast agents (e.g. SonoVue microbubbles comprising sulfur hexafluoride, Optison microbubbles comprising an albumin shell and octafluoropropane gas core, Levovist microbubbles comprising a lipid/galactose shell and an air core, Perflexane lipid microspheres comprising perfluorocarbon microbubbles, and Perflutren lipid microspheres comprising octafluoropropane encapsulated in an outer lipid shell), magnetic resonance imaging (MRI) contrast agents (e.g., gadodiamide, gadobenic acid, gadopentetic acid, gadoteridol, gadofosveset, gadoversetamide, gadoxetic acid), and radiocontrast agents, such as for computed tomography (CT), radiography, or fluoroscopy (e.g., diatrizoic acid, metrizoic acid, iodamide, iotalamic acid, ioxitalamic acid, ioglicic acid, acetrizoic acid, iocarmic acid, methiodal, diodone, metrizamide, iohexol, ioxaglic acid, iopamidol, iopromide, iotrolan, ioversol, iopentol, iodixanol, iomeprol, iobitridol, ioxilan, iodoxamic acid, iotroxic acid, ioglycamic acid, adipiodone, iobenzamic acid, iopanoic acid, iocetamic acid, sodium iopodate, tyropanoic acid, and calcium iopodate). As with many of the standard procedures associated with the practice of the invention, skilled artisans will be aware of additional labels that can be used.

Genotyping may also comprise sequencing nucleic acids from a sample collected from an individual using any convenient sequencing protocol. Sequencing platforms that can be used include but are not limited to: pyrosequencing, sequencing-by-synthesis, single-molecule sequencing, second-generation sequencing, nanopore sequencing, sequencing by ligation, or sequencing by hybridization. Preferred sequencing platforms are those commercially available from Illumina (RNA-Seq) and Helicos (Digital Gene Expression or “DGE”). “Next generation” sequencing methods include, but are not limited to those commercialized by: 1) 454/Roche Lifesciences including but not limited to the methods and apparatus described in Margulies et al., Nature (2005) 437:376-380 (2005); and U.S. Pat. Nos. 7,244,559; 7,335,762; 7,211,390; 7,244,567; 7,264,929; 7,323,305; 2) Helicos BioSciences Corporation (Cambridge, MA) as described in U.S. application Ser. No. 11/167,046, and U.S. Pat. Nos. 7,501,245; 7,491,498; 7,276,720; and in U.S. Patent Application Publication Nos. US20090061439; US20080087826; US20060286566; US20060024711; US20060024678; US20080213770; and US20080103058; 3) Applied Biosystems (e.g. SOLID sequencing); 4) Dover Systems (e.g., Polonator G.007 sequencing); 5) Illumina as described U.S. Pat. Nos. 5,750,341; 6,306,597; and 5,969,119; and 6) Pacific Biosciences as described in U.S. Pat. Nos. 7,462,452; 7,476,504; 7,405,281; 7,170,050; 7,462,468; 7,476,503; 7,315,019; 7,302,146; 7,313,308; and US Application Publication Nos. US20090029385; US20090068655; US20090024331; and US20080206764. All references are herein incorporated by reference. Such methods and apparatuses are provided here by way of example and are not intended to be limiting.

Genetic testing services exist, which provide full genome sequencing using massively parallel sequencing. Massively parallel sequencing is described e.g. in U.S. Pat. No. 5,695,934, entitled “Massively parallel sequencing of sorted polynucleotides,” and U.S. Patent Application Publication No. 2010/0113283 A1, entitled “Massively multiplexed sequencing.” Massively parallel sequencing typically involves obtaining DNA representing an entire genome, fragmenting it, and obtaining millions of random short sequences, which are assembled by mapping them to a reference genome sequence. Commercial services are available that are capable of genotyping approximately 1 million sequences for a fixed fee.

Genetic analysis can be carried out with a variety of methods that do not involve massively parallel random sequencing. For example, a commercially available MassARRAY system can be used. This system uses matrix-assisted laser desorption ionization time-of-flight mass spectrometry (MALDI-TOF MS) coupled with single-base extension PCR for high-throughput multiplex detection of mutations. Another commercial system, the Illumina Golden Gate assay, generates mutation-specific PCR products that are subsequently hybridized to beads either on a solid matrix or in solution. Three oligonucleotides are synthesized for each mutant: two allele specific oligonucleotides (ASOs) that distinguish the mutated sequence, and a locus specific sequence (LSO) just downstream of the mutation site. The ASO and LSO sequences also contain target sequences for a set of universal primers, while each LSO also contains a particular address sequences (the “illumicode”) complementary to sequences attached to beads.

### Data Analysis

In some embodiments, one or more pattern recognition methods can be used in automating analysis of genetic data and generating a predictive model. The predictive models and/or algorithms can be provided in a machine-readable format and may be used to correlate non-coding somatic mutations identified in a patient by genotyping with the risk of preterm birth. Generating the predictive model may comprise, for example, the use of an algorithm or classifier. In some embodiments, a deep learning model based on a deep residual neural network (RNN) or a deep convolution neural network (CNN) is used to predict tissue-specific chromatin structure for a given genomic sequence and to identify somatic mutations that alter the chromatin structure in a deleterious manner that promotes preterm birth. The deep learning model can be used for genome-wide computation of DEEP or DEEP+ scores for somatic mutations (see Examples).

### System and Computer Implemented Methods for Predicting the Risk of Preterm Birth

In another aspect, a computer implemented method is provided for predicting the risk of preterm birth for an individual. The computer performs steps comprising: a) receiving genome sequencing data for an individual; b) identifying non-coding somatic mutations associated with preterm birth present in the individual from the genome sequencing data, wherein the individual has a plurality of non-coding somatic mutations selected from Table 1; c) calculating a composite DEEP+ score for the non-coding somatic mutations detected in the individual by genotyping using a database described herein, wherein the composite DEEP+ score indicates the risk of preterm birth for the individual; and d) displaying information regarding the risk of preterm birth for the individual. In certain embodiments, the computer implemented method further comprises storing the information regarding the risk of preterm birth for the individual in a database.

In certain embodiments, the computer implemented method further comprises calculating a composite BEAR risk score for the non-coding somatic mutations detected in the individual by genotyping using a database described herein, wherein the composite BEAR risk score indicates the risk of preterm birth for the individual.

In another aspect, a database comprising DEEP+ scores for a plurality of non-coding somatic mutations associated with preterm birth is provided. In some embodiments, the database comprises or consists of DEEP+ scores for non-coding somatic mutations selected from Table 1. In some embodiments, the database further comprises BEAR risk scores for the plurality of non-coding somatic mutations associated with preterm birth. A computer implemented method may utilize such a database to calculate a composite DEEP+ score and/or BEAR risk score for the non-coding somatic mutations detected by genotyping.

In a further aspect, a computer implemented method is provided for predicting therapeutic responsiveness of an individual to treatment with progestin for preterm labor. The computer performs steps comprising: a) receiving genome sequencing data for an individual; b) identifying one or more non-coding somatic mutations associated with risk of preterm birth in one or more genes selected from the group consisting of AHNAK, ANTXR2, ATP1B1, ATP2B4, CALM2, CAPZA2, CAV1, CDC42EP3, CITED2, CNN1, CORO1C, CPQ, CSDE1, DCN, DPP6, DPYSL3, DST, DSTN, DYNC1LI2, FHL1, FILIP1L, GSN, HADH, HSPB8, IGFBP7, ITM2B, KANK2, KCNMA1, LDB2, MAP4, MBNL1, MFAP5, MGP, MSRB3, MYH11, MYLK, MYO1C, NR2F2, PALLD, PARVA, PBX1, PGR, PKD2, PLN, PLS3, PPP1R12B, PRUNE2, PTN, RAP2C, RSPO3, SERINC1, SH3BGRL, SLMAP, SORBS1, SPARCL1, SUN1, SVIL, SYNPO2, TACC1, TBC1D1, TCEAL4, TES, TIMP2, TJP1, TMEM123, TNS1, TPM1, YAP1, and YWHAZ; c) calculating a composite DEEP+ score for the one or more non-coding somatic mutations detected in the individual by genotyping using a database described herein, wherein if the composite DEEP+ score is above a reference threshold value, the individual is identified as a non-responder who will not benefit from the treatment with the progestin, and wherein if the composite DEEP+ score is below a reference threshold value, the individual is identified as a responder who will benefit from the treatment with the progestin; and d) displaying information regarding whether the individual is identified as a non-responder or a responder.

In certain embodiments, the computer implemented method further comprises calculating a composite BEAR risk score for the non-coding somatic mutations detected in the individual by genotyping using the database, wherein if the composite BEAR risk score is above a reference threshold value, the individual is identified as a non-responder who will not benefit from the treatment with the progestin, and wherein if the composite BEAR risk score is below a reference threshold value, the individual is identified as a responder who will benefit from the treatment with the progestin.

In certain embodiments, the computer implemented method further comprises storing the information regarding whether the individual is identified as a non-responder or a responder in a database.

The computer implemented methods can be implemented in digital electronic circuitry, or in computer software, firmware, or hardware. The disclosed and other embodiments can be implemented as one or more computer program products, i.e., one or more modules of computer program instructions encoded on a computer readable medium for execution by, or to control the operation of, a data processing apparatus. The computer readable medium can be a machine-readable storage device, a machine-readable storage substrate, a memory device, a composition of matter effecting a machine-readable propagated signal, or any combination thereof.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

In a further aspect, a system for performing a computer implemented method, as described, is provided. Such a system includes a computer containing a processor, a storage component (i.e., memory), a display component, and other components typically present in general purpose computers. The storage component stores information accessible by the processor, including instructions that may be executed by the processor and data that may be retrieved, manipulated or stored by the processor.

The storage component includes instructions. For example, the storage component may include instructions for predicting the risk of preterm birth in the individual based on analysis of genomic sequencing data stored therein. Alternatively or additionally, the storage component may include instructions for predicting therapeutic responsiveness of an individual to treatment with progestin for preterm labor based on analysis of the genomic sequencing data stored therein. The computer processor is coupled to the storage component and configured to execute the instructions stored in the storage component in order to receive genome sequencing data and analyze the data according to one or more algorithms (e.g., deep residual neural network or deep convolutional neural network), as described herein. The display component may display information regarding the risk of preterm birth for the individual and/or display information regarding whether the individual is identified as a non-responder or a responder.

The storage component may be of any type capable of storing information accessible by the processor, such as a hard-drive, memory card, ROM, RAM, DVD, CD-ROM, USB Flash drive, write-capable, and read-only memories. The processor may be any well-known processor, such as processors from Intel Corporation. Alternatively, the processor may be a dedicated controller such as an ASIC.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data.

In certain embodiments, the processor and storage component may comprise multiple processors and storage components that may or may not be stored within the same physical housing. For example, some of the instructions and data may be stored on removable CD-ROM and others within a read-only computer chip. Some or all of the instructions and data may be stored in a location physically remote from, yet still accessible by, the processor. Similarly, the processor may comprise a collection of processors which may or may not operate in parallel.

### Kits

Kits are also provided for carrying out the methods described herein. In some embodiments, the kit comprises software for carrying out a computer implemented method for predicting the risk of preterm birth for an individual based on detection of non-coding somatic mutations associated with preterm labor, as described herein. In some embodiments, the kit comprises software for carrying out a computer implemented method for predicting therapeutic responsiveness of an individual to treatment with progestin for preterm labor based on detection of non-coding somatic mutations associated with preterm labor, as described herein. In some embodiments, the kit further comprises a container for collecting a DNA sample from an individual. The kit may also include reagents for purifying, genotyping, and/or sequencing a DNA sample.

In addition, the kits may further include (in certain embodiments) instructions for practicing the subject methods. These instructions may be present in the subject kits in a variety of forms, one or more of which may be present in the kit. For example, instructions may be present as printed information on a suitable medium or substrate, e.g., a piece or pieces of paper on which the information is printed, in the packaging of the kit, in a package insert, and the like. Another form of these instructions is a computer readable medium, e.g., diskette, compact disk (CD), flash drive, and the like, on which the information has been recorded. Yet another form of these instructions that may be present is a website address which may be used via the internet to access the information at a removed site.

### Pharmaceutical Compositions

Agents for treating preterm labor (e.g., RKI-1447, bisindolylmaleimide-ix, TBBt, LY294002, URMC099, Bosutinib, and SB-203580) can be formulated into pharmaceutical compositions optionally comprising one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A composition of the invention can also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the agent, or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (BASF, Mount Olive, New Jersey); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the agent (e.g., when contained in a drug delivery system) in the composition will vary depending on a number of factors but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

The amount of any individual excipient in the composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, NJ (1998), and Kibbe, A.H., Handbook of Pharmaceutical Excipients, 3rd Edition, American Pharmaceutical Association, Washington, D.C., 2000.

The compositions encompass all types of formulations and in particular those that are suited for injection, e.g., powders or lyophilates that can be reconstituted with a solvent prior to use, as well as ready for injection solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional preferred compositions include those for oral, intravenous, intramuscular, vaginal, intrathecal, intraspinal, or localized delivery such as by injection into the myometrium to inhibit contractions.

The pharmaceutical preparations herein can also be housed in a syringe, an implantation device, or the like, depending upon the intended mode of delivery and use. Preferably, the compositions comprising the agent are in unit dosage form, meaning an amount of a conjugate or composition of the invention appropriate for a single dose, in a premeasured or pre-packaged form.

The compositions herein may optionally include one or more additional agents, such other drugs for treating preterm labor or pain, or other medications. For example, compounded preparations may include at least one agent for treating preterm labor (e.g., RKI-1447, bisindolylmaleimide-ix, TBBt, LY294002, URMC099, Bosutinib, or SB-203580) and one or more other drugs for treating preterm labor or pain, including, without limitation, progestin, analgesics such as opioids (e.g., fentanyl, Nubain (nalbuphine), morphine, and Stadol (butorphanol)), nitrous oxide, and general or local anesthetics (e.g., pudendal block, epidural block (e.g., bupivacaine and ropivacaine), spinal block (e.g., bupivacaine, fentanyl, and morphine), combined spinal-epidural (CSE) block, or paracervical block).

### Administration

At least one therapeutically effective cycle of treatment with a composition comprising an agent for inhibiting preterm labor (e.g., RKI-1447, bisindolylmaleimide-ix, TBBt, LY294002, URMC099, Bosutinib, and SB-203580) will be administered to a subject for treatment of preterm labor. By “therapeutically effective dose or amount” of an agent for inhibiting preterm labor (e.g., RKI-1447, bisindolylmaleimide-ix, TBBt, LY294002, URMC099, Bosutinib, and SB-203580) is intended an amount that, when administered brings about a positive therapeutic response, such as decreasing myometrial contraction and preventing preterm birth. The exact amount required will vary from subject to subject, depending on the species, age, and general condition of the subject, the severity of the condition being treated, the particular type of agent employed to inhibit preterm labor, the mode of administration, and the like. An appropriate “effective” amount in any individual case may be determined by one of ordinary skill in the art using routine experimentation, based upon the information provided herein.

In certain embodiments, multiple therapeutically effective doses of compositions comprising an agent for inhibiting preterm labor (e.g., RKI-1447, bisindolylmaleimide-ix, TBBt, LY294002, URMC099, Bosutinib, and SB-203580), and/or one or more other therapeutic agents, such as one or more other drugs for treating preterm labor or pain or other medications. For example, compounded preparations may include at least one agent for inhibiting preterm labor (e.g., RKI-1447, bisindolylmaleimide-ix, TBBt, LY294002, URMC099, Bosutinib, and SB-203580) and one or more other drugs for treating preterm labor or pain, including, without limitation, progestin, analgesics such as opioids (e.g., fentanyl, Nubain (nalbuphine), morphine, and Stadol (butorphanol)), nitrous oxide, general or local anesthetics (e.g., pudendal block, epidural block (e.g., bupivacaine and ropivacaine), spinal block (e.g., bupivacaine, fentanyl, and morphine), combined spinal-epidural (CSE) block, or paracervical block), or other medications will be administered. The compositions comprising the agent for inhibiting preterm labor (e.g., RKI-1447, bisindolylmaleimide-ix, TBBt, LY294002, URMC099, Bosutinib, and SB-203580) are typically, although not necessarily, administered orally, via injection (subcutaneously, intravenously, intramuscularly, or vaginally), by infusion, topically, or locally. Additional modes of administration are also contemplated, such as intrathecal, intraspinal, or localized delivery such as by injection into the myometrium, and so forth.

The preparations according to the invention are also suitable for local treatment. For example, compositions comprising an agent for inhibiting preterm labor (e.g., RKI-1447, bisindolylmaleimide-ix, TBBt, LY294002, URMC099, Bosutinib, and SB-203580) may be administered by injection into the myometrium. The particular preparation and appropriate method of administration can be chosen to target the agent to the myometrium to inhibit myometrial contraction. Local treatment may avoid some side effects of systemic therapy.

The pharmaceutical preparation can be in the form of a liquid solution or suspension immediately prior to administration, but may also take another form such as a syrup, cream, ointment, tablet, capsule, powder, gel, matrix, suppository, or the like. The pharmaceutical compositions comprising an agent for inhibiting preterm labor (e.g., RKI-1447, bisindolylmaleimide-ix, TBBt, LY294002, URMC099, Bosutinib, and SB-203580) and/or other agents may be administered using the same or different routes of administration in accordance with any medically acceptable method known in the art.

In another embodiment, the pharmaceutical compositions comprising the agent for inhibiting preterm labor (e.g., RKI-1447, bisindolylmaleimide-ix, TBBt, LY294002, URMC099, Bosutinib, and SB-203580) and/or other drugs for treating preterm labor or pain, and/or other agents are in a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

Those of ordinary skill in the art will appreciate which conditions the agent for inhibiting preterm labor (e.g., RKI-1447, bisindolylmaleimide-ix, TBBt, LY294002, URMC099, Bosutinib, and SB-203580) can effectively treat. The actual dose to be administered will vary depending upon the age, weight, and general condition of the subject as well as the severity of the condition being treated, the judgment of the health care professional, and conjugate being administered. Therapeutically effective amounts can be determined by those skilled in the art, and will be adjusted to the particular requirements of each particular case.

In certain embodiments, multiple therapeutically effective doses of a composition comprising an agent for inhibiting preterm labor (e.g., RKI-1447, bisindolylmaleimide-ix, TBBt, LY294002, URMC099, Bosutinib, and SB-203580) will be administered according to a daily dosing regimen or intermittently. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, once a week, every other week, and so forth. For example, in some embodiments, a composition comprising the agent for inhibiting preterm labor (e.g., RKI-1447, bisindolylmaleimide-ix, TBBt, LY294002, URMC099, Bosutinib, or SB-203580) will be administered once-weekly, twice-weekly or thrice-weekly for an extended period of time, such as for 1, 2, 3, 4, 5, 6, 7, 8 . . . 10 . . . 15 . . . 24 weeks, and so forth. By “twice-weekly” or “two times per week” is intended that two therapeutically effective doses of the agent in question is administered to the subject within a 7 day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7 day period, allowing for a minimum of 48 hours between doses and a maximum of 72 hours between doses. For purposes of the present invention, this type of dosing is referred to as “intermittent” therapy. In accordance with the methods of the present invention, a subject can receive intermittent therapy (i.e., once-weekly, twice-weekly or thrice-weekly administration of a therapeutically effective dose) for one or more weekly cycles until the desired therapeutic response is achieved. The agents can be administered by any acceptable route of administration as noted herein below. The amount administered will depend on the potency of the agent for inhibiting preterm labor (e.g., RKI-1447, bisindolylmaleimide-ix, TBBt, LY294002, URMC099, Bosutinib, or SB-203580) and/or other agents administered, the magnitude of the effect desired, and the route of administration.

The agent (again, preferably provided as part of a pharmaceutical preparation) can be administered alone or in combination with one or more other therapeutic agents, such as other agents for treating preterm labor or pain, or other medications used to treat a particular condition or disease according to a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Preferred compositions are those requiring dosing no more than once a day.

The agent for inhibiting preterm labor (e.g., RKI-1447, bisindolylmaleimide-ix, TBBt, LY294002, URMC099, Bosutinib, or SB-203580) can be administered prior to, concurrent with, or subsequent to other agents. If provided at the same time as other agents, the agent for inhibiting preterm labor (e.g., RKI-1447, bisindolylmaleimide-ix, TBBt, LY294002, URMC099, Bosutinib, or SB-203580) can be provided in the same or in a different composition. Thus, the agent for inhibiting preterm labor (e.g., RKI-1447, bisindolylmaleimide-ix, TBBt, LY294002, URMC099, Bosutinib, or SB-203580) and one or more other agents can be presented to the individual by way of concurrent therapy. By “concurrent therapy” is intended administration to a subject such that the therapeutic effect of the combination of the substances is caused in the subject undergoing therapy. For example, concurrent therapy may be achieved by administering a dose of a pharmaceutical composition comprising the agent for inhibiting preterm labor (e.g., RKI-1447, bisindolylmaleimide-ix, TBBt, LY294002, URMC099, Bosutinib, or SB-203580) and a dose of a pharmaceutical composition comprising at least one other agent, such as another drug for treating an infection, which in combination comprise a therapeutically effective dose, according to a particular dosing regimen. Similarly, the agent for inhibiting preterm labor (e.g., RKI-1447, bisindolylmaleimide-ix, TBBt, LY294002, URMC099, Bosutinib, or SB-203580) and one or more other therapeutic agents can be administered in at least one therapeutic dose. Administration of the separate pharmaceutical compositions can be performed simultaneously or at different times (i.e., sequentially, in either order, on the same day, or on different days), as long as the therapeutic effect of the combination of these substances is caused in the subject undergoing therapy.

Toxicity can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in further optimizing and/or defining a therapeutic dosage range and/or a sub-therapeutic dosage range (e.g., for use in humans). The exact formulation, route of administration and dosage can be chosen by the individual physician in view of the patient's condition.

### Utility

The methods described herein are useful for predicting the risk of preterm birth for an individual based on personalized tissue-specific genotyping to detect non-coding somatic mutations associated with preterm labor. The disclosed methods are also useful for predicting therapeutic responsiveness of an individual to treatment with progestin for preterm labor and selecting an appropriate treatment regimen.

### EXAMPLES OF NON-LIMITING ASPECTS OF THE DISCLOSURE

Aspects, including embodiments, of the present subject matter described above may be beneficial alone or in combination, with one or more other aspects or embodiments. Without limiting the foregoing description, certain non-limiting aspects of the disclosure numbered 1-58 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method for genome-wide identification of non-coding somatic mutations associated with preterm birth, the method comprising:
a) providing a database comprising epigenomic correlation data for associations between non-coding somatic mutations and chromatin structural changes associated with myometrial transition to preterm labor based on genome-wide epigenomic screening of a population of patients experiencing preterm birth;
b) generating a deep learning model to compute the probability that a given genomic sequence has an open chromatin structure; and
c) using the deep learning model to identify non-coding somatic mutations associated with the myometrial transition to preterm labor, wherein a non-coding somatic mutation is considered to contribute to risk of preterm birth if an allelic change from its corresponding reference wild-type allele to the somatic mutation results in an alteration in predicted chromatin openness based on the deep learning model.
   2 The method of aspect 1, wherein the deep learning model uses a deep residual neural network or deep convolutional neural network.
   3. The method of aspect 2, further comprising calculating deep estimation from epigenome prediction plus (DEEP+) scores for each non-coding somatic mutation that is identified as contributing to the risk of preterm birth for an individual.
   4. The method of aspect 3, wherein the DEEP+ scores are used in combination with genome-wide association study (GWAS) risk scores for each non-coding somatic mutation to determine the risk of preterm birth for an individual.
   5. The method of aspect 3 or 4, wherein the DEEP+ scores are used in combination with haploinsufficiency scores for each non-coding somatic mutation to determine the risk of preterm birth for an individual.
   6. The method of any one of aspects 3 to 5, wherein the DEEP+ scores are used in combination with myometrial transcriptomic profiling data to determine the risk of preterm birth for an individual.
   7. The method of aspect 6, further comprising using a Bayesian estimation for altered regulation (BEAR) model to calculate a BEAR composite risk score for each non-coding somatic mutation that is identified as contributing to risk of preterm birth, wherein the BEAR model uses a mixture Gaussian model of the distribution of the DEEP+ scores, the GWAS risk scores, the gene haploinsufficiency scores, and the myometrial transcriptomic profiling data to calculate the BEAR composite risk score, wherein the BEAR composite risk score is used to determine the risk of preterm birth for an individual.
   8. The method of any one of aspects 1 to 7, wherein the patient is European or African American.
   9. The method of any one of aspects 1 to 8, wherein one or more of the non-coding somatic mutations are in genes that regulate myometrial muscle relaxation or inflammatory responses.
   10. The method of any one of aspects 1 to 9, wherein the non-coding somatic mutations are in an intronic genomic region, a promoter, a 5′ untranslated region (5′ UTR), a 3′ untranslated region (3′ UTR), an exonic genomic region, an intergenic genomic region, or a genomic region encoding a non-coding RNA.
   11. The method of any one of aspects 1 to 10, wherein the non-coding somatic mutations comprise at least one insertion, deletion, or single-nucleotide variant.
   12. The method of any one of aspects 1 to 11, wherein the epigenomic correlation data comprises assay for transposase-accessible chromatin sequencing (ATAC-Seq) data.
   13. A method of predicting risk of preterm birth for an individual, the method comprising:
a) obtaining a biological sample from the individual;
b) genotyping one or more cells in the biological sample to determine if the individual has one or more non-coding somatic mutations associated with risk of preterm birth; and
c) calculating a composite DEEP+ score for the one or more non-coding somatic mutations associated with risk of preterm birth detected by genotyping, wherein the composite DEEP+ score indicates the risk of preterm birth.
   14. The method of aspect 13, wherein the composite DEEP+ score is used in combination with genome-wide association study (GWAS) risk scores for each non-coding somatic mutation associated with risk of preterm birth detected by genotyping to determine the risk of preterm birth.
   15. The method of aspect 13 or 14, wherein the composite DEEP+ score is used in combination with haploinsufficiency scores for each non-coding somatic mutation associated with risk of preterm birth detected by genotyping to determine the risk of preterm birth.
   16. The method of any one of aspects 13 to 15, wherein the composite DEEP+ score is used in combination with myometrial transcriptomic profiling data to determine the risk of preterm birth.
   17. The method of aspect 16, further comprising using a Bayesian estimation for altered regulation (BEAR) model to calculate a composite BEAR risk score for each non-coding somatic mutation associated with risk of preterm birth detected by genotyping, wherein the BEAR model uses a mixture Gaussian model of the distribution of the DEEP+ scores, the GWAS risk scores, the gene haploinsufficiency scores, and the myometrial transcriptomic profiling data to calculate the composite BEAR risk score, wherein the composite BEAR risk score indicates the risk of preterm birth.
   18. The method of any one of aspects 13 to 17, wherein the non-coding somatic mutations are in an intronic genomic region, a promoter, a 5′ untranslated region (5′ UTR), a 3′ untranslated region (3′ UTR), an exonic genomic region, an intergenic genomic region, or a genomic region encoding a non-coding RNA.
   19. The method of any one of aspects 13 to 18, wherein the non-coding somatic mutations comprise at least one insertion, deletion, or single-nucleotide variant.
   20. The method of any one of aspects 13 to 19, wherein the one or more non-coding somatic mutations associated with risk of preterm birth comprise one or more non-coding somatic mutations selected from Table 1.
   21. The method of any one of aspects 13 to 20, wherein said genotyping comprises sequencing at least part of a genome of a cell from the biological sample.
   22. The method of aspect 21, wherein said genotyping comprises sequencing the whole genome of a cell from the biological sample.
   23. The method of any one of aspects 13 to 22, wherein the biological sample is a myometrium sample.
   24. The method of any one of aspects 13 to 23, further comprising treating the individual to reduce risk of preterm birth if the composite DEEP+ score indicates the individual is at risk of preterm birth.
   25. The method of any one of aspects 13 to 23, further comprising treating the individual to reduce risk of preterm birth if the composite BEAR risk score indicates the individual is at risk of preterm birth.
   26. The method of aspect 24 or 25, wherein said treating comprises administering progestin to the individual.
   27. The method of any one of aspects 13 to 26, further comprising predicting non-responsiveness of the individual to treatment with progestin based on identifying one or more non-coding somatic mutations in one or more genes selected from the group consisting of AHNAK, ANTXR2, ATP1B1, ATP2B4, CALM2, CAPZA2, CAV1, CDC42EP3, CITED2, CNN1, CORO1C, CPQ, CSDE1, DCN, DPP6, DPYSL3, DST, DSTN, DYNC1LI2, FHL1, FILIP1L, GSN, HADH, HSPB8, IGFBP7, ITM2B, KANK2, KCNMA1, LDB2, MAP4, MBNL1, MFAP5, MGP, MSRB3, MYH11, MYLK, MYO1C, NR2F2, PALLD, PARVA, PBX1, PGR, PKD2, PLN, PLS3, PPP1R12B, PRUNE2, PTN, RAP2C, RSPO3, SERINC1, SH3BGRL, SLMAP, SORBS1, SPARCL1, SUN1, SVIL, SYNPO2, TACC1, TBC1D1, TCEAL4, TES, TIMP2, TJP1, TMEM123, TNS1, TPM1, YAP1, and YWHAZ.
   28. A database comprising deep estimation from epigenome prediction plus (DEEP+) scores for a plurality of non-coding somatic mutations associated with preterm birth.
   29. The database of aspect 28, wherein the database comprises or consists of DEEP+ scores for non-coding somatic mutations selected from Table 1.
   30. The database of aspect 28 or 29, wherein the database further comprises or consists of Bayesian estimation for altered regulation (BEAR) risk scores for the plurality of non-coding somatic mutations associated with preterm birth.
   31. A computer implemented method for predicting risk of preterm birth for an individual, the computer performing steps comprising:
a) receiving genome sequencing data for an individual;
b) identifying non-coding somatic mutations associated with preterm birth present in the individual from the genome sequencing data, wherein the individual has a plurality of non-coding somatic mutations selected from Table 1;
c) calculating a composite deep estimation from epigenome prediction plus (DEEP+) risk score for the non-coding somatic mutations detected in the individual by genotyping using the database of any one of aspects 28 to 30, wherein the composite DEEP+ score indicates the risk of preterm birth for the individual; and
d) displaying information regarding the risk of preterm birth for the individual.
   32. The computer implemented method of aspect 31, further comprising calculating a composite Bayesian estimation for altered regulation (BEAR) risk score for the non-coding somatic mutations detected in the individual by genotyping using the database, wherein the composite BEAR risk score indicates the risk of preterm birth for the individual.
   33. The computer implemented method of aspect 31 or 32, further comprising storing the information regarding the risk of preterm birth for the individual in a database.
   34. A system for predicting the risk of preterm birth for an individual using the computer implemented method of any one of aspects 31 to 33, the system comprising:
a) a storage component for storing data, wherein the storage component has instructions for predicting the risk of preterm birth for an individual based on analysis of the genome sequencing data stored therein;
b) a computer processor for processing the genome sequencing data using one or more algorithms, wherein the computer processor is coupled to the storage component and configured to execute the instructions stored in the storage component in order to receive the inputted genome sequencing data and analyze the data according to the computer implemented method of any one of aspects 31 to 33; and
c) a display component for displaying the information regarding the risk of preterm birth for the individual.
   35. A non-transitory computer-readable medium comprising program instructions that, when executed by a processor in a computer, causes the processor to perform the computer implemented method of any one of aspects 31 to 33.
   36. A kit comprising the non-transitory computer-readable medium of aspect 35 and instructions for predicting the risk of preterm birth for an individual.
   37. A method of predicting therapeutic responsiveness of an individual to treatment with progestin for preterm labor, the method comprising:
a) obtaining a biological sample from the individual;
b) genotyping one or more cells in the biological sample to determine if the individual has one or more non-coding somatic mutations associated with risk of preterm birth in one or more genes selected from the group consisting of AHNAK, ANTXR2, ATP1B1, ATP2B4, CALM2, CAPZA2, CAV1, CDC42EP3, CITED2, CNN1, CORO1C, CPQ, CSDE1, DCN, DPP6, DPYSL3, DST, DSTN, DYNC1LI2, FHL1, FILIP1L, GSN, HADH, HSPB8, IGFBP7, ITM2B, KANK2, KCNMA1, LDB2, MAP4, MBNL1, MFAP5, MGP, MSRB3, MYH11, MYLK, MYO1C, NR2F2, PALLD, PARVA, PBX1, PGR, PKD2, PLN, PLS3, PPP1R12B, PRUNE2, PTN, RAP2C, RSPO3, SERINC1, SH3BGRL, SLMAP, SORBS1, SPARCL1, SUN1, SVIL, SYNPO2, TACC1, TBC1D1, TCEAL4, TES, TIMP2, TJP1, TMEM123, TNS1, TPM1, YAP1, and YWHAZ; and
c) calculating a composite DEEP+ score for the one or more non-coding somatic mutations detected in the individual by genotyping using the database of any one of aspects 28 to 30, wherein if the composite DEEP+ score is above a reference threshold value, the individual is identified as a non-responder who will not benefit from the treatment with the progestin, and wherein if the composite DEEP+ score is below a reference threshold value, the individual is identified as a responder who will benefit from the treatment with the progestin.
   38. The method of aspect 37, further comprising calculating a composite Bayesian estimation for altered regulation (BEAR) risk score for the non-coding somatic mutations detected in the individual by genotyping using the database, wherein if the composite BEAR risk score is above a reference threshold value, the individual is identified as a non-responder who will not benefit from the treatment with the progestin, and wherein if the composite BEAR risk score is below a reference threshold value, the individual is identified as a responder who will benefit from the treatment with the progestin.
   39. The method of aspect 37 or 38, further comprising administering progestin to the individual if the individual is identified as a responder.
   40. The method of any one of aspects 37 to 39, wherein said genotyping comprises sequencing at least part of a genome of a cell from the biological sample.
   41. The method of aspect 40, wherein said genotyping comprises sequencing the whole genome of a cell from the biological sample.
   42. The method of any one of aspects 37 to 41, wherein the biological sample is a myometrium sample.
   43. A computer implemented method for predicting therapeutic responsiveness of an individual to treatment with progestin for preterm labor, the computer performing steps comprising:
a) receiving genome sequencing data for an individual;
b) identifying one or more non-coding somatic mutations associated with risk of preterm birth in one or more genes selected from the group consisting of AHNAK, ANTXR2, ATP1B1, ATP2B4, CALM2, CAPZA2, CAV1, CDC42EP3, CITED2, CNN1, CORO1C, CPQ, CSDE1, DCN, DPP6, DPYSL3, DST, DSTN, DYNC1LI2, FHL1, FILIP1L, GSN, HADH, HSPB8, IGFBP7, ITM2B, KANK2, KCNMA1, LDB2, MAP4, MBNL1, MFAP5, MGP, MSRB3, MYH11, MYLK, MYO1C, NR2F2, PALLD, PARVA, PBX1, PGR, PKD2, PLN, PLS3, PPP1R12B, PRUNE2, PTN, RAP2C, RSPO3, SERINC1, SH3BGRL, SLMAP, SORBS1, SPARCL1, SUN1, SVIL, SYNPO2, TACC1, TBC1D1, TCEAL4, TES, TIMP2, TJP1, TMEM123, TNS1, TPM1, YAP1, and YWHAZ;
c) calculating a composite DEEP+ score for the one or more non-coding somatic mutations detected in the individual by genotyping using the database of any one of aspects 28 to 30, wherein if the composite DEEP+ score is above a reference threshold value, the individual is identified as a non-responder who will not benefit from the treatment with the progestin, and wherein if the composite DEEP+ score is below a reference threshold value, the individual is identified as a responder who will benefit from the treatment with the progestin; and
d) displaying information regarding whether the individual is identified as a non-responder or a responder.
   44. The computer implemented method of aspect 43, further comprising calculating a composite Bayesian estimation for altered regulation (BEAR) risk score for the non-coding somatic mutations detected in the individual by genotyping using the database, wherein if the composite BEAR risk score is above a reference threshold value, the individual is identified as a non-responder who will not benefit from the treatment with the progestin, and wherein if the composite BEAR risk score is below a reference threshold value, the individual is identified as a responder who will benefit from the treatment with the progestin.
   45. The computer implemented method of aspect 43 or 44, further comprising storing the information regarding whether the individual is identified as a non-responder or a responder in a database.
   46. A system for predicting therapeutic responsiveness of an individual to treatment with progestin for preterm labor using the computer implemented method of any one of aspects 43 to 45, the system comprising:
a) a storage component for storing data, wherein the storage component has instructions for predicting the therapeutic responsiveness of an individual to treatment with progestin based on analysis of the genome sequencing data stored therein;
b) a computer processor for processing the genome sequencing data using one or more algorithms, wherein the computer processor is coupled to the storage component and configured to execute the instructions stored in the storage component in order to receive the inputted genome sequencing data and analyze the data according to the computer implemented method of any one of aspects 43 to 45; and
c) a display component for displaying the information regarding whether the individual is identified as a responder or a non-responder.
   47. A non-transitory computer-readable medium comprising program instructions that, when executed by a processor in a computer, causes the processor to perform the computer implemented method of any one of aspects 43 to 45.
   48. A kit comprising the non-transitory computer-readable medium of aspect 47 and instructions for predicting the therapeutic responsiveness of an individual to treatment with progestin.
   49. A method of treating preterm labor in a pregnant female subject, the method comprising administering a therapeutically effective amount of a composition comprising RKI-1447, bisindolylmaleimide-ix, TBBt, LY294002, URMC099, Bosutinib, or SB-203580 to the pregnant female subject.
   50. The method of aspect 49, wherein the composition is administered orally, intravenously, intramuscularly, or vaginally.
   51. The method of aspect 49, wherein the composition is administered locally to the myometrium.
   52. The method of any one of aspects 49 to 51, wherein the pregnant female subject is having preterm labor or identified as having a risk of preterm labor.
   53. The method of any one of aspects 49 to 52, wherein multiple cycles of treatment are administered to the pregnant female subject.
   54. The method of aspect 53, wherein the composition is administered daily or intermittently.
   55. The method of aspect 53 or 54, wherein the composition is administered to the pregnant female subject during pregnancy beginning at 16 to 20 weeks of gestation.
   56. The method of any one of aspects 53 to 55, wherein the composition is administered to the pregnant female subject until delivery.
   57. A composition comprising RKI-1447, bisindolylmaleimide-ix, TBBt, LY294002, URMC099, Bosutinib, or SB-203580 for use in a method of treating preterm labor.
   58. The composition of aspect 57, further comprising a pharmaceutically acceptable excipient.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

### Example 1

### An Integrative Analysis of Noncoding Mutations Identifies the Druggable Genome in Preterm Birth

### Introduction

Genome-wide agnostic identification of at-risk loci in spontaneous preterm birth has been performed in past decades, but only identified very few genomic loci. For example, the latest genome-wide association study (GWAS) scanned more than 15 million genomic loci among 43,568 study participants, and only identified three significant loci in preterm birth, each with moderate effect sizes (risk odds ratios, ORs less than 1.2) (Zhang et al., 2017). We reasoned that in a typical GWAS framework, disease associations are indirectly inferred from allele frequency imbalances between case and control groups. However, if we are able to directly quantify the mutational consequence of every variant across the entire genome relevant to a disease of interest, aggregating the molecular information with GWAS risk profiles would significantly expand our view of the genetic architecture in a particular complex disease. Moreover, because >90% of disease-associated loci fall in non-coding genomic regions (Boix et al., 2021; Corradin and Scacheri, 2014; Schaub et al., 2012), integrating tissue-specific epigenomes and existing GWAS frameworks would provide deeper mechanistic insights.

In this work, we devised deep-learning and Bayesian graphical models to integrate the epigenome of the term pregnancy myometrium and large-scale genomes from patient cohorts experiencing spontaneous preterm birth and normal term birth. This integrative framework enabled us to directly quantify every base change across the genome for its effect on perturbing the myometrial epigenome and its contribution to risk of preterm birth. Our genome scan agnostically identified genes associated with spontaneous preterm labor, most of which have not been previously described for their etiological roles in this condition. These novel genes displayed functional specificities towards smooth muscle relaxation and inflammatory responses. Using functional genomic data from human tissues, we validated their significant involvement in spontaneous preterm labor. We leveraged these newly identified genes for therapeutic development, where we longitudinally recruited a cohort of pregnant women with past history of preterm delivery who responded to or did not respond to progestin therapy in the form of weekly injections of 17-hydroxyprogesterone caproate. We observed that variants specific to these identified genes in personal genomes predicted personal responses to the treatment, i.e., whether pregnancies ended prematurely or not. To develop new therapeutic strategies, we screened more than 4,000 small molecules for their potential effects on treating preterm labor, and identified novel candidate molecules affecting the identified genes from our model. We experimentally validated their therapeutic potential in vitro. Overall, our integrative machine-learning framework revealed genetic architecture in spontaneous preterm birth, identified its druggable genome for personalized therapy, and unveiled novel molecules with therapeutic potential for treating preterm birth.

### Results

### Developing an Integrative Model to Identify Genes in Spontaneous Preterm Birth

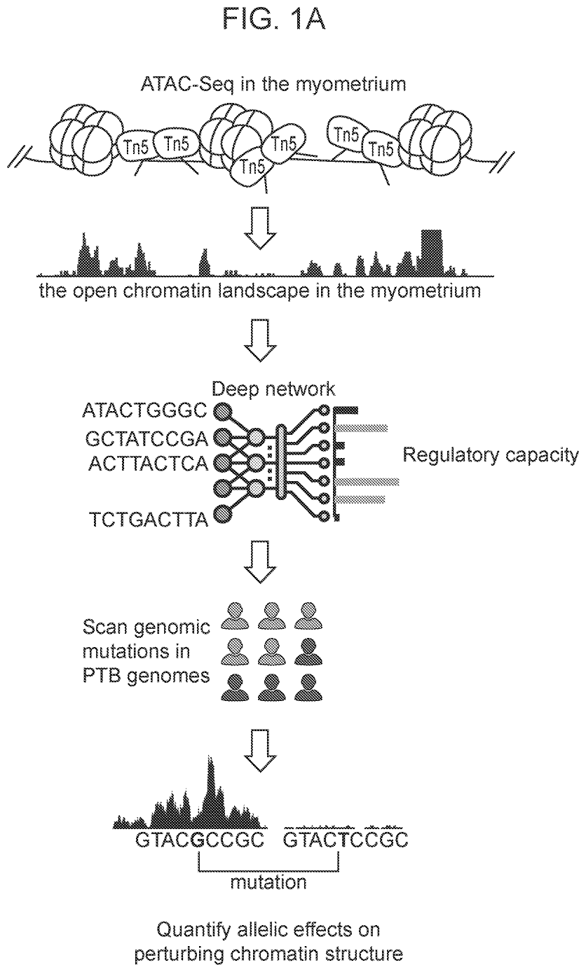

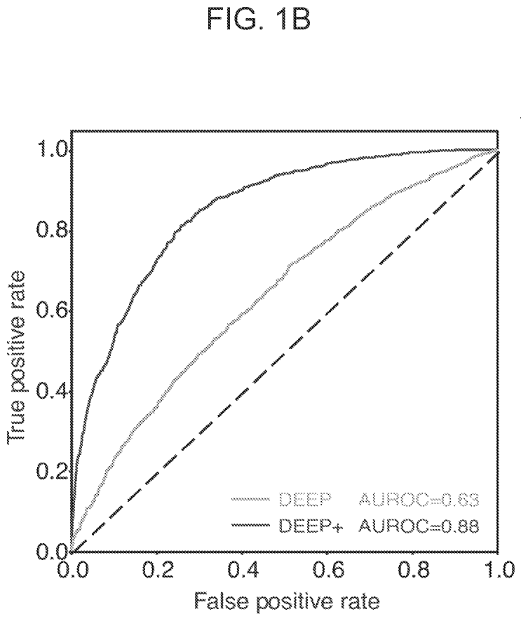

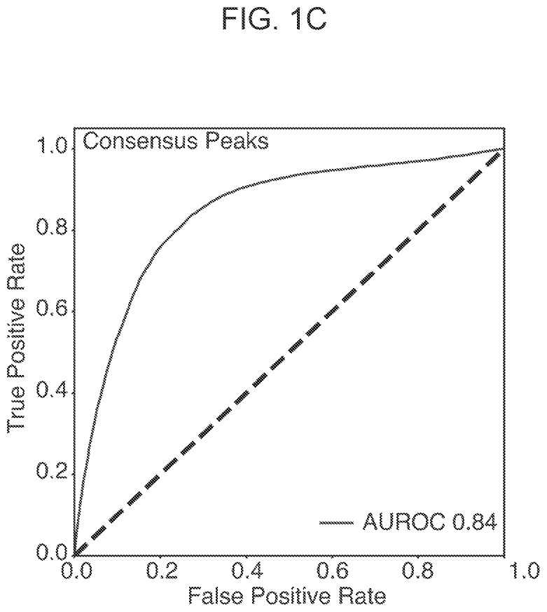

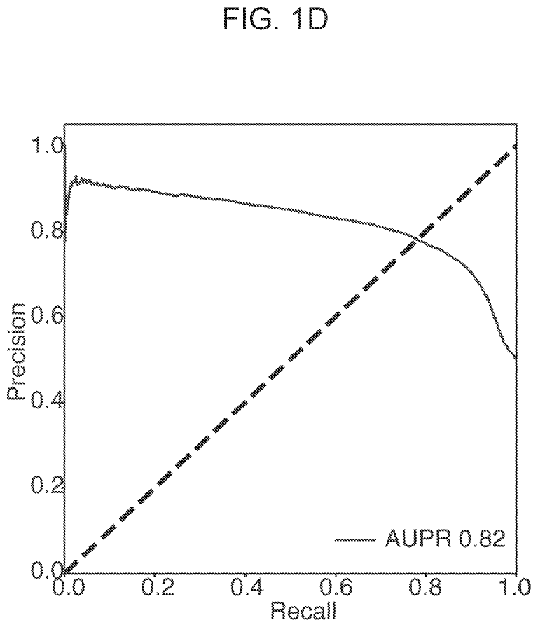

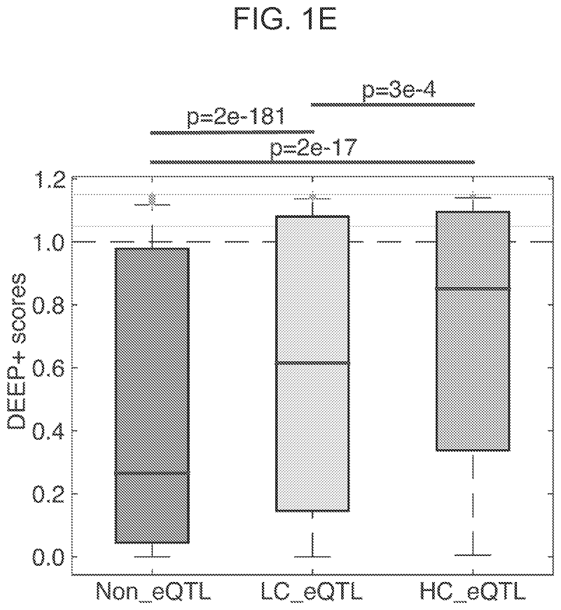

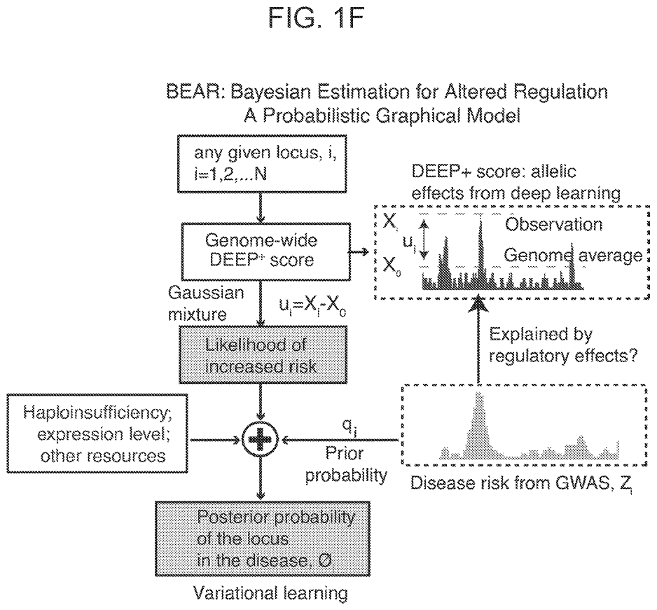

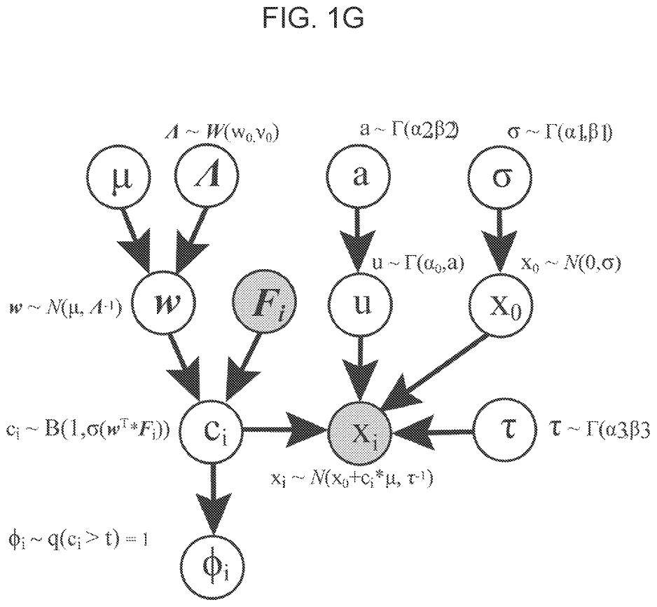

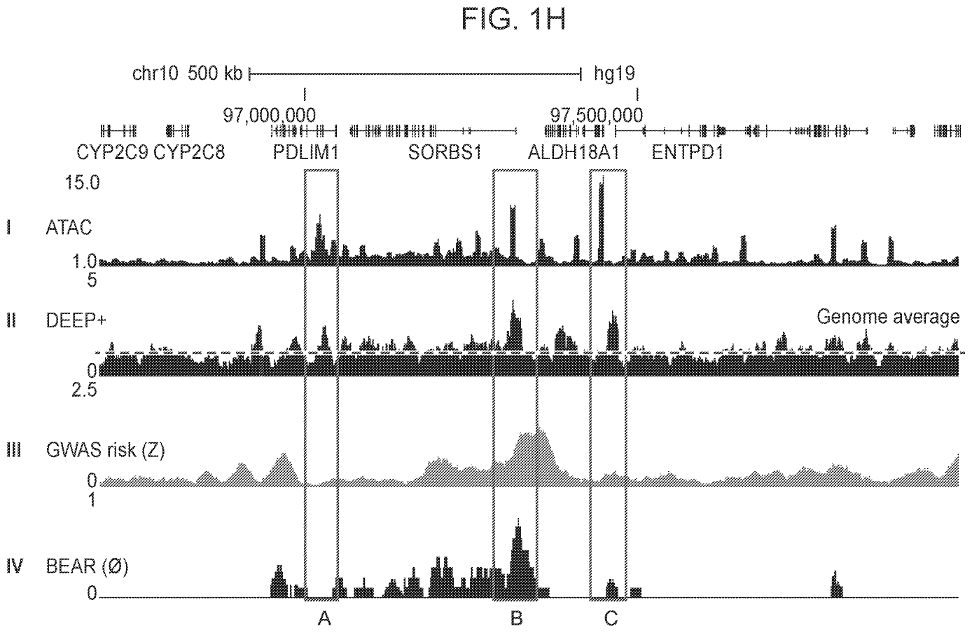

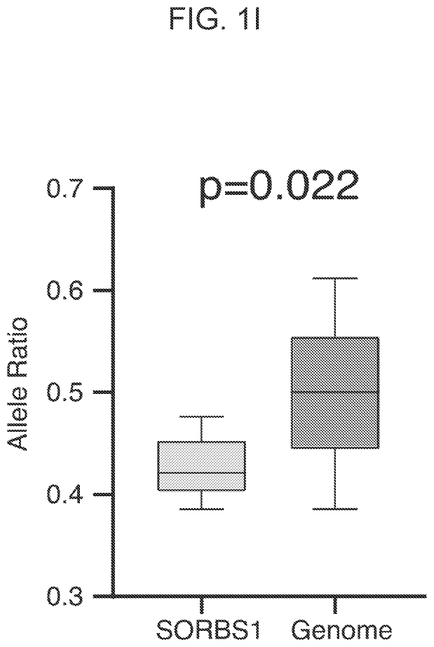

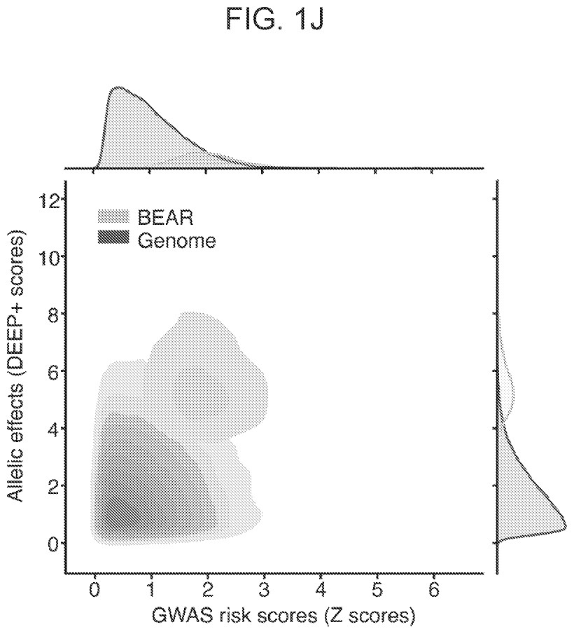

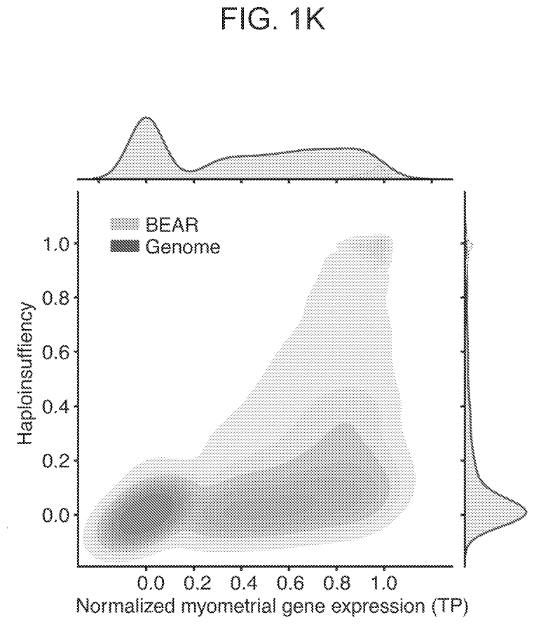

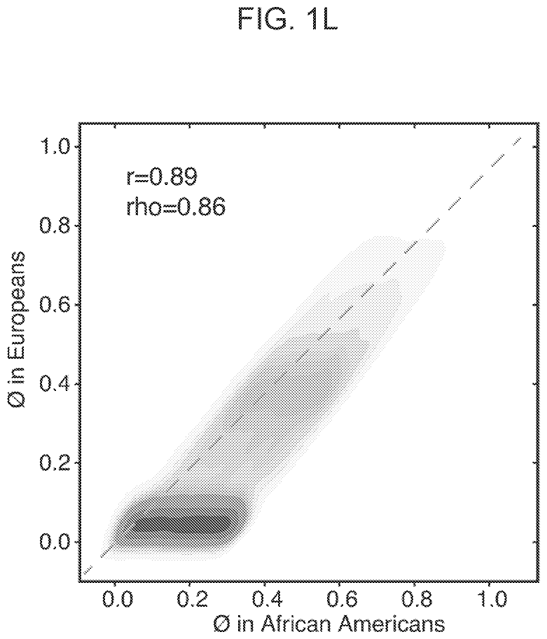

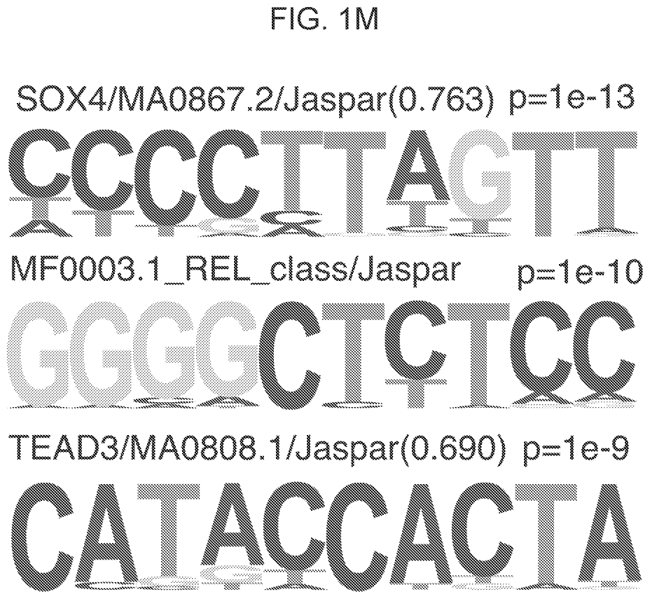

We previously implemented a deep-learning model, which scans the entire human genome to identify single base changes that alter tissue-specific epigenomes (Wang and Li, 2020). We have demonstrated its clinical utility by studying the non-coding genome in prostate cancer. The deep learning model, DEEP (Deep Estimation from Epigenome Prediction), learns regulatory code in the entire collection of open chromatin regions in a given tissue from ATAC-Seq data, enabling genome-wide prediction of tissue-specific chromatin accessibility for any given genomic sequences (Wang and Li, 2020). The model is then used to score each genomic mutation to quantify the allelic effect on the predicted chromatin accessibility changes from a reference to an alternative allele. Extreme scores indicate significant changes in chromatin architecture resulting from regulatory mutations (FIG. 1A). Compared with a conventional association-based framework (e.g. QTL analysis), the deep-learning framework directly quantifies mechanistic effects of genomic mutations.

We utilized published ATAC-Seq data to build the deep learning model, and then performed blind test on ATAC-Seq data generated from our independently collected clinical samples. We first analyzed existing ATAC-Seq data from pregnant myometrium samples at term (three biological replicates), which were each delivered by Cesarean section (non-laboring, 38-42 gestational weeks, see Methods and Materials) (Wu et al., 2020). We considered the term pregnancy myometrium in this analysis because of the shared molecular characteristics between term and preterm labor, including an activation of inflammatory factors, infiltration of the myometrium by neutrophils and macrophages, and functional progesterone withdrawal as well as an array of downstream cascade events initiating the labor onset (Mendelson et al., 2019). All these shared mechanisms have allowed us to leverage term labor samples as a reference for fine mapping genetic variants in preterm labor patient cohorts, which disrupt reference genetic elements, dysregulate labor timing and thereby predispose individuals to increased preterm labor risk.

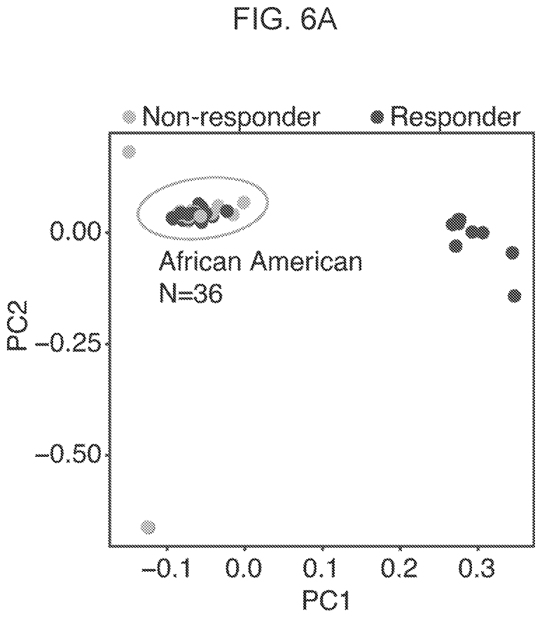

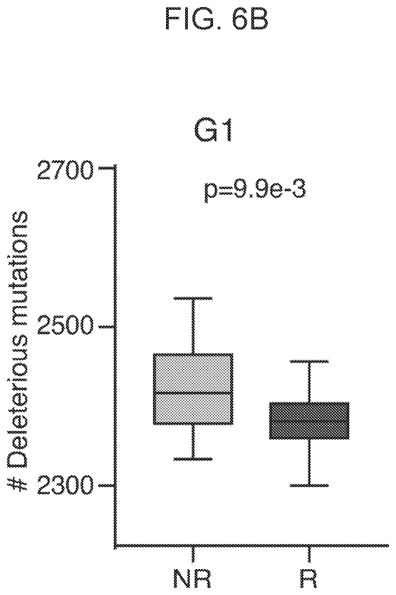

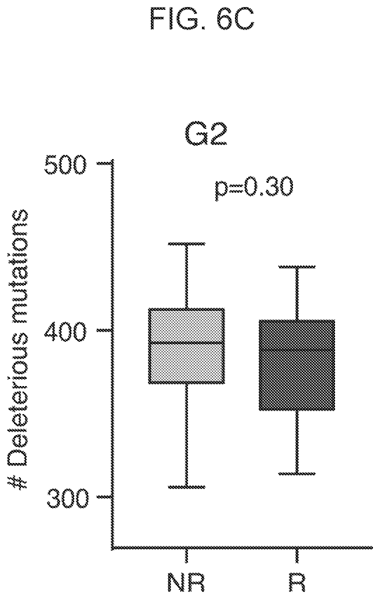

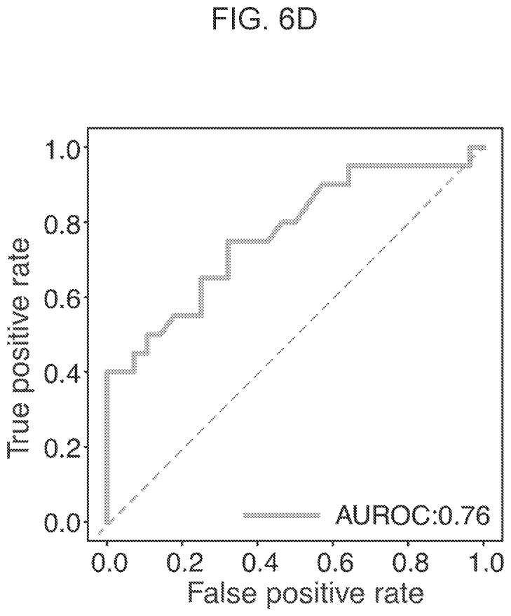

We performed independent QC and confirmed the high quality of this dataset. Specifically, we leveraged our standardized and benchmarked pipeline to process the ATAC-Seq data and identified 46,059 high-confidence ATAC-Seq peaks that passed our QC (FIG. 6). These peak regions were shared among three biological replicates, hallmarking the characteristic genome-wide localization of active regulatory elements in the myometrium at term. We used the myometrium ATAC-Seq data to decipher the myometrial regulatory code based on our deep learning model (FIG. 1A). We upgraded our original deep-learning model (DEEP) to an advanced version (DEEP+) by replacing the original deep convolutional neural network (Wang and Li, 2020) with the deep residual neural network in our model (He et al., 2016). DEEP+ demonstrated substantially enhanced performance in predicting myometrial open chromatin regions comparing to the original DEEP model based on a series of blind tests (FIG. 1B).

To ensure robust performance and generalizability of our deep learning model, we collected five snap frozen pregnant myometrium samples at term. Our ATAC-seq identified on average 80,500 open chromatin peaks in each sample (Methods and Materials). Testing the pre-trained DEEP+ model (trained with previously published data, described above) on these independently collected new clinical samples, we observed that the model had satisfying performance with AUROC=0.84 (area under the receiver operating characteristic curve, FIG. 1C and AUPR=0.82 (area under the precision recall curve, FIG. 1D) when considering ATAC-Seq peaks shared across all the five independently collected clinical samples. Therefore, testing on our independently collected samples confirmed that the deep learning model has indeed learned the sequence characteristics encoding open chromatin regions in the myometrium at term. In other words, for a given genomic locus with two alleles, the model can readily predict chromatin accessibility associated with each allele, and their difference naturally quantifies the allelic effects on perturbing local chromatin architecture in the myometrium (FIG. 1A). To associate chromatin alterations with gene expression changes, we leveraged the trained model to score common variants across the genomes in the GTEx (the Genotype-Tissue Expression) cohort (Consortium, 2020), and then compared our predictions against the high-confidence expression quantitative loci (HC-eQTLs) in the uterus (129 uterus samples, eQTLs annotated by GTEx). We observed that the uterus HC-eQTLs displayed the strongest allelic effects predicted by the DEEP+ model (FIG. 1E, Methods and Materials), indicating the allelic effects of these eQTLs on perturbing their local chromatin accessibility in the uterus. This comparison demonstrated that the highly scored alleles in our model were more likely to affect gene expression.

As illustrated in FIG. 1A, we used DEEP+ to score the allelic effect on perturbing myometrial chromatin accessibility at term for each of ~13 million genomic variants in a GWAS cohort of spontaneous preterm birth including 43,568 study participants with European ancestry (Zhang et al., 2017). The risk odds ratios at each locus have been calculated in this dataset after stratifying fine population structure, which were subsequently standardized into risk Z-scores. To associate mutational consequences with the risk of developing spontaneous preterm birth, we constructed a Bayesian graphical model, BEAR (Bayesian Estimation for Altered Regulation). This allowed us to integrate mutational allelic effects (DEEP+ scores) in the myometrium at term with the genomic risk profile from the GWAS data for spontaneous preterm birth (Zhang et al., 2017). The Bayesian model calculates posterior probabilities to quantify the confidence of any given loci in spontaneous preterm birth conditioned on its allelic effects on altering myometrial epigenome at term (DEEP+ scores), its GWAS risk for preterm birth, as well as information from other genomic resources. Specifically, we considered a confident locus in spontaneous preterm birth if its allele received an increased GWAS risk score accompanied with elevated DEEP+ scores (perturbing myometrial chromatin architecture at pregnant term). Because we examined allelic effects on gene regulation, we expected that impactful variants would affect genes that are dosage-sensitive, and we also integrated the widely used pLI scores to approximate gene haploinsufficiency (Karczewski et al., 2020; Lek et al., 2016) in the Bayesian model. We also considered confident loci if they affected active genes in the myometrium at pregnant term. Taken together, constructing such an evidence-based integrative framework in a hierarchical Bayesian structure has enabled effective removal of nuisance signals from a typical genome scan, directly revealing mechanistic insights in the molecular etiologies of preterm birth.

The BEAR model design is shown in FIG. 1F, where we essentially used multi-dimensional genomic information as evidence to boost confidence on a given genomic locus for its association with spontaneous preterm labor. The Bayesian implementation of the model is shown in FIG. 1G, where we used mixture Gaussian to model the distribution of DEEP+ scores across the genome, aggregating GWAS risk profiles (Z-scores), the myometrial transcriptome at term (Wu et al., 2020), and gene haploinsufficiency scores (Huang et al., 2010) as model priors (Methods and Materials). It is important to note that unlike a GWAS framework where genomic loci are strongly affected by linkage disequilibrium (LD), the calculation of DEEP+ scores was independent at each genomic locus without being affected by LD. Therefore, the DEEP+ score at a given locus is its own allelic effect on changing local chromatin accessibility. Because the hierarchical Bayesian structure had no closed form, we used variational learning (Wang and Blei, 2013) as an approximate solution to derive the posterior probability (Ø, the BEAR scores) of any given locus for its association with spontaneous preterm birth conditioned on DEEP+ scores(S), GWAS risk Z scores (Z), myometrial expression at term (E) and gene haploinsufficiency scores (H) (FIG. 1G). Note that although the BEAR model utilized the GWAS signal to associate genomic loci with risk, the identified loci from BEAR should be largely understood as mechanistic given DEEP+ scores directly quantifying variant consequences on gene regulation.

We used the BEAR model to scan the entire genome, and computed BEAR scores (Ø) at each locus. For example, ATAC-Seq data in the non-laboring term myometrium indicated open chromatin structure in the promoter region of SORBS1 (the ATAC-Seq peak, panel I, FIG. 1H). Our DEEP+ model assigned high deleteriousness scores (far above the genome average, panel II, FIG. 1H) for SNPs in this promoter region, suggesting that a cluster of genomic variants in the SORBS1 promoter strongly affected the chromatin openness. To confirm these chromatin effects on gene expression, we examined myometrium RNA-seq data at term pregnancy (Wu et al., 2020). In the RNA-seq data, we identified all the heterozygous alleles in the SORBS1 gene body which were linked with the identified SORBS1 promoter alleles. We observed that these linked alleles were more likely to display allele-specific expression (allelic ratio=0.427±0.032), a significant deviation from the expected genomic background with a ratio at 0.5 (P=0.022, Wilcoxon rank-sum test, FIG. 1I). Therefore, this observation suggested that these identified risk of preterm birth alleles within the promoter likely manifested their chromatin effects on affecting SORBS1 expression. Likewise, these promoter alleles also displayed substantially increased GWAS risk for spontaneous preterm birth (panel III, FIG. 1H), which together with SORBS1 haploinsufficiency and its high expression in the normal pregnant term myometrium collectively boosted our confidence on implicating SORBS1 as a candidate locus (the promoter region) with altered chromatin landscape in spontaneous preterm labor. We analyzed previously published myometrial transcriptome data (Chan et al., 2014) and indeed observed that SORBS1 was significantly down-regulated in the myometrium upon labor onset, suggesting a potential role of SORBS1 in regulating labor timing. This was further supported by its REACTOME annotation of smooth muscle contraction (Fabregat et al., 2018). In contrast, inconsistent signals across multiomic datasets at a given locus will reduce BEAR scores (box A, B and C, FIG. 1H), reflecting a lower confidence level. This example illustrates how our BEAR model could uncover novel genes in the disease by aggregating multi-omics datasets.

Given the genome-wide distribution of BEAR scores (Ø), we compared the score distribution in gene promoters and putative enhancers in uterine smooth muscle cells (curated by FANTOM5 (Andersson et al., 2014)) and observed that variants in promoters tended to receive greater BEAR scores than those in enhancers (P=2.2e-96). Therefore, at the genome-wide scale, gene promoter regions were more likely to harbor at-risk loci associated with this disease. Among the ~13 million genomic variants in the GWAS cohort (Zhang et al., 2017), we identified 1,079 loci for their associations with spontaneous preterm birth, reflecting the top 0.01% among all genomic loci (corresponding to their BEAR scores, Ø>0.9325). These loci were uniquely mapped onto 315 protein-coding genes (Table 1), whose functions will be characterized below.

For the purpose of model checking, we examined these confidently identified loci at different levels to ensure that they indeed behaved as expected in our Bayesian model design. At the variant level, we confirmed that the identified loci displayed significantly increased GWAS risk for spontaneous preterm birth (x-axis, FIG. 1J, relative to the genome background) and elevated mutational effects on perturbing myometrial chromatin structure at term (y-axis, FIG. 1J, relative to the genome background). At the gene level, we confirmed that the variants we identified affected genes with extreme dosage sensitivity (reflected by their increased haploinsufficiency scores, y-axis, FIG. 1K) and elevated expression in the non-laboring term myometrium (x-axis, FIG. 1K). These observations confirmed that the BEAR model worked as anticipated and the identified variants as well as their associated genes were consistent with our initial model design.

BEAR captures shared molecular etiologies between Europeans and African Americans The BEAR model was constructed from the GWAS cohort of European individuals, and we next asked whether the model output would be affected by population ancestry. We particularly noted that the model output, posterior probability Ø (FIGS. 1F and 1G), is a composite score optimized by iterative computational trade-offs among mutational deleteriousness (DEEP+ scores), GWAS risk scores, haploinsufficiency, myometrial gene expression as well as many other model hyperparameters (FIGS. 1F and 1G). Therefore, our model output score Ø was expected to be robust against small allele frequency fluctuations among populations. We implemented the model on an independent GWAS cohort of African Americans (BBC, the Boston Birth Cohort), where 461 women were spontaneous preterm birth cases and 1,035 matched controls (Hong et al., 2017). We computed mutational deleteriousness (DEEP+ scores) across BBC genomes, which were further integrated with the original BBC GWAS risk profiles (the standardized Z scores) by the BEAR model, generating the posterior probability scores for each of the BBC loci quantifying their posterior probabilities for their contribution to spontaneous preterm labor. We compared the Ø scores between the European and African-American cohorts, and observed a strong correlation between the populations (R=0.89, Pearson's correlation, and rho=0.86, Spearman's rank correlation, FIG. 1L). This indicates that BEAR is able to infer the shared molecular etiologies between different population ancestry that are possibly masked when only interpreting from GWAS data. The BBC cohort also served as an independent validation for our studies using the European cohort.

### Functional Characterization of the Identified Loci in Spontaneous Preterm Birth

Given the large GWAS cohort of European individuals and the overall genome concordance with the BBC cohort, we further functionally characterized the identified 1,079 loci using the European cohort as a reference. Because these loci demonstrated strong allelic effects on altering their local chromatin structure (FIGS. 1J, 1K), we asked whether these alterations would affect the recognition of transcription factors (TFs) to their binding sites. We centered each of the 1,079 BEAR loci and extended 50 bp sequences in both upstream and downstream directions. We then performed motif enrichment analysis on this set of 101-bp sequences and we observed significant enrichment of binding motifs of SOX4 (P=1e-13), NF-kB (P=1e-10) and TEAD3 (P=1e-9) (FIG. 1M). SOX4 and NF-kB are known to regulate of smooth muscle contraction (Bethin et al., 2003; Khanjani et al., 2011), especially NF-kB playing a central role in regulating labor timing (Khanjani et al., 2011; Mendelson et al., 2019). This observation suggested that the identified alleles likely perturb genomic occupancies by SOX4, NF—KB and TEAD3 through remodeling local chromatin accessibility in the pregnant myometrium.

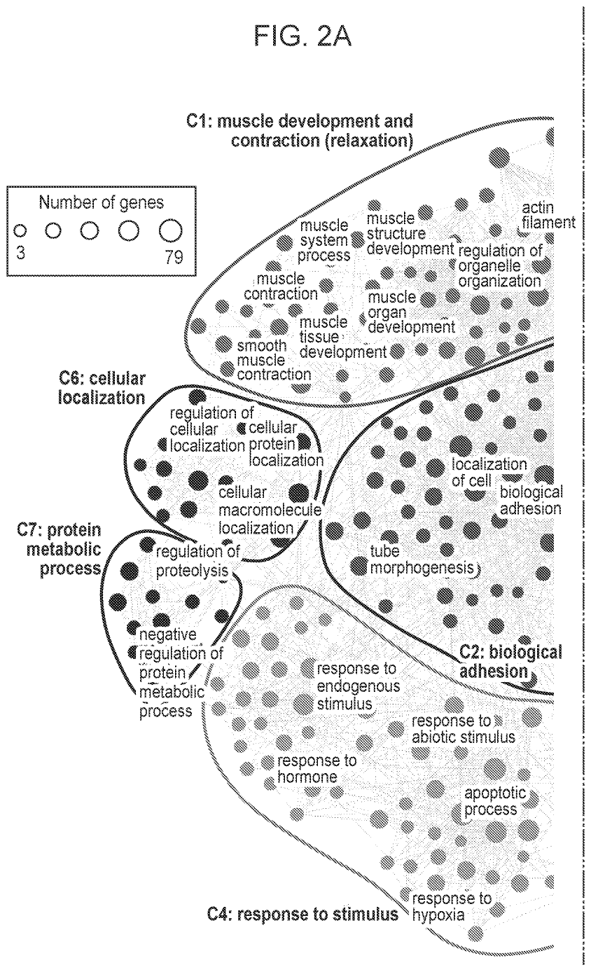

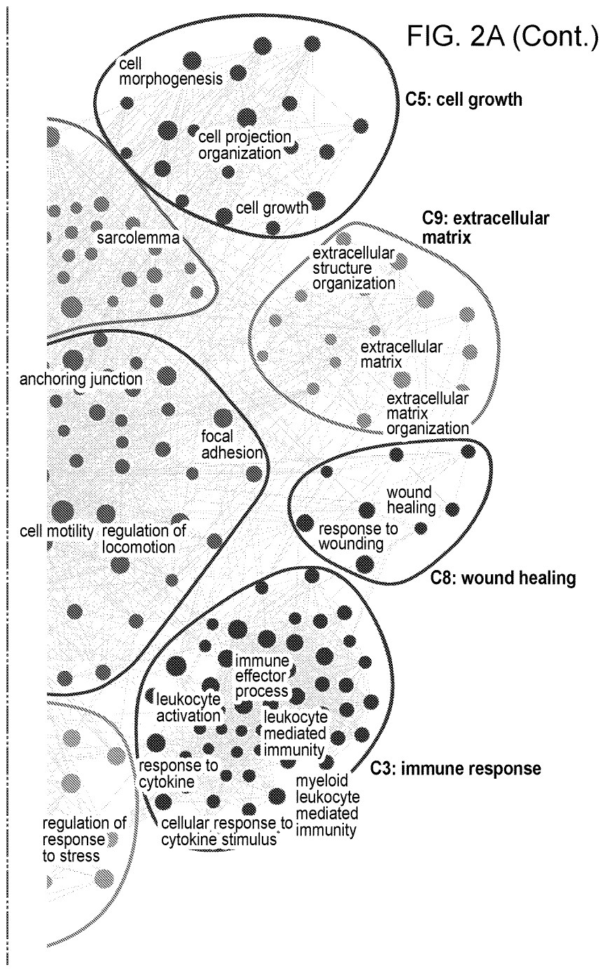

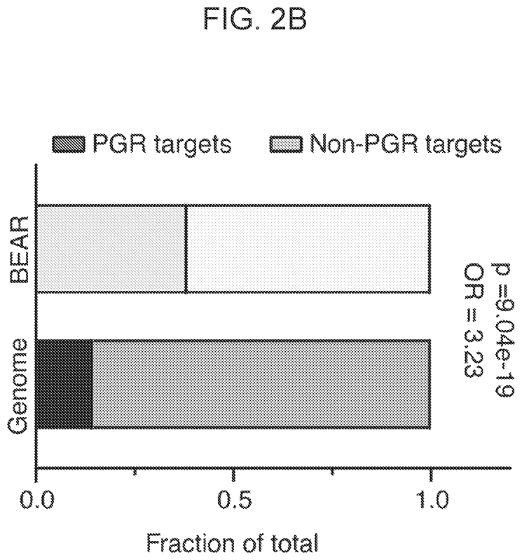

As described above, the 1,079 high-confidence loci were mapped onto 315 protein-coding genes. We observed their overall enrichment in several main functional categories, particularly in regulating muscle contraction (FDR=8.71e-12, FIG. 2A and Table 2), extracellular matrix function (FDR=3.24e-16, FIG. 2A and Table 2) and response to cytokine (FDR=2.76e-6, FIG. 2A and Table 2). Because the identified genes had increased myometrial expression, to ensure that the observed functional enrichment cannot be merely explained by gene expression levels, we perform an additional control experiment by randomly sampling genes matching the size and myometrial gene expression with our identified genes (Methods and Materials). As expected, the specific enrichments for muscle (P=0.42) and inflammatory functions cannot be observed (P>0.9) on the randomly sampled genes, confirming functional specificities of our model output. The functional category of regulating muscle contraction is heralded by CALD1 (Caldesmon 1) together with many myosin proteins (MYH9, MYLK, MYL6, MYL9), the calmodulin factor CALM2, the potassium calcium channel proteins (KCNMA1, KCNMB1) as well as many other factors. We particularly note CALD1, which represses smooth muscle contraction via inhibiting actomyosin ATPase, and therefore maintains myometrial relaxation during pregnancy (Rehman et al., 2003). Overall, these enriched functional categories are consistent with myometrial phenotype at term prior to labor onset, which serves as further evidence for the role of the identified genes in regulating labor timing. Given the central role of the progesterone receptor (PGR) in regulating labor timing (Amini et al., 2019; Nadeem et al., 2017) by modulating contractile and inflammatory proteins at the transition from quiescence to labor (Tan et al., 2012), we further asked whether these agnostically identified genes by BEAR were in fact convergent onto PGR-mediated regulatory network. We examined ChIP-Seq data targeting PGR in the non-laboring term myometrial samples from a previous study (Wu et al., 2020). We re-analyzed the ChIP-Seq data, performed additional QC and considered 4,860 confident ChIP-Seq peaks shared between two biological replicates (see Supplementary Materials), revealing genome-wide PGR occupancy in this particular physiological condition. We observed that 44.1% (139/315) of the identified BEAR genes were directly targeted by PGR in the myometrium at term, compared with the genome average of 11.2% (P=9.04e-19, Fisher's exact test, FIG. 2B). Therefore, our genome scan demonstrated that disrupting the PGR-mediated regulatory network in the myometrium is strongly associated with spontaneous preterm birth.

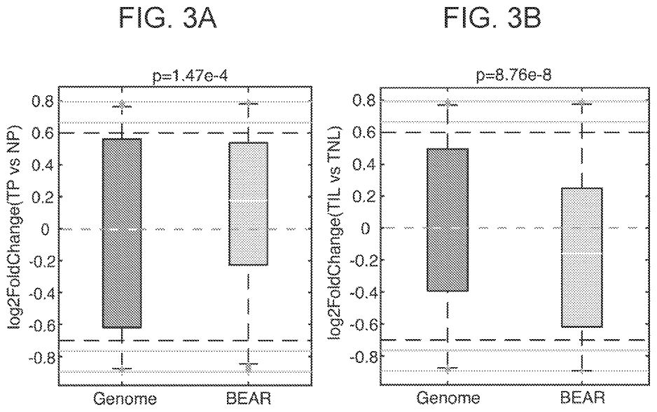

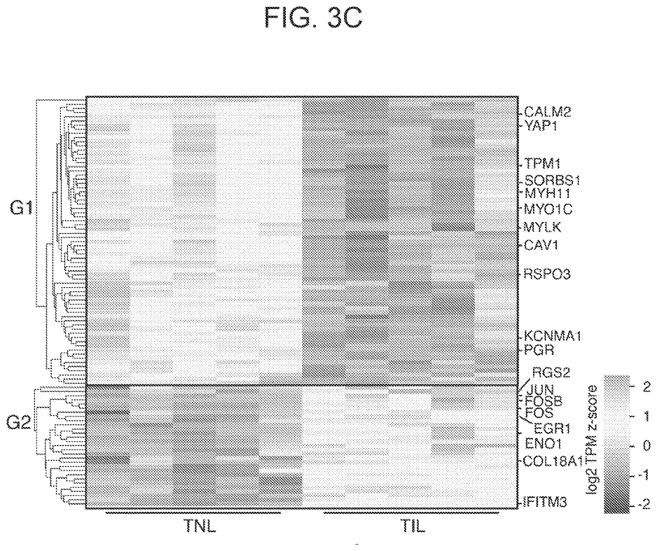

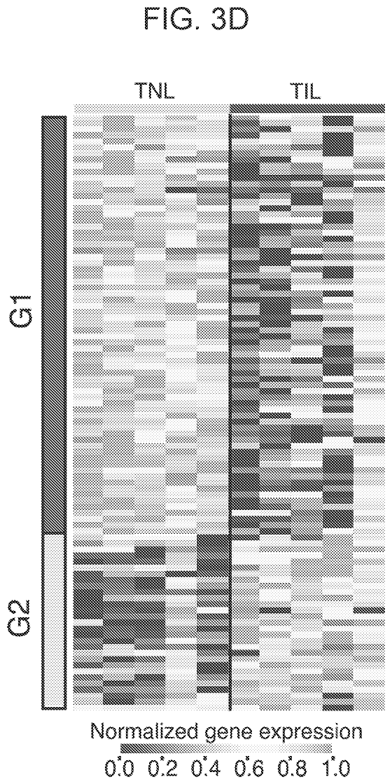

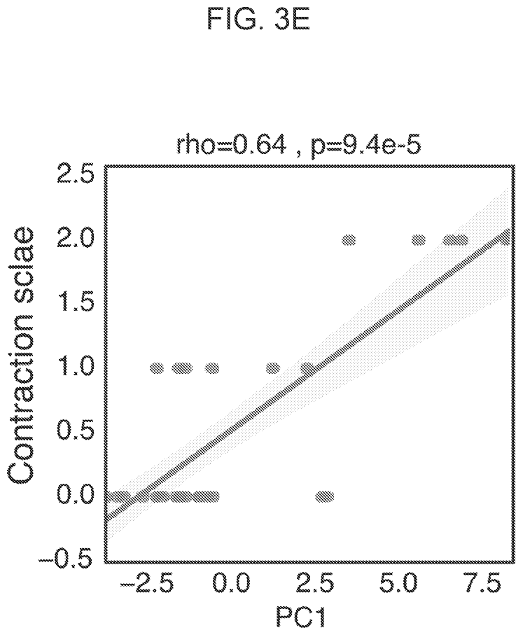

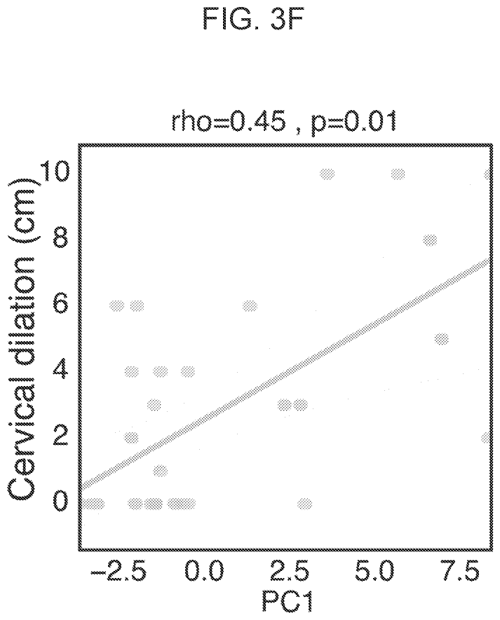

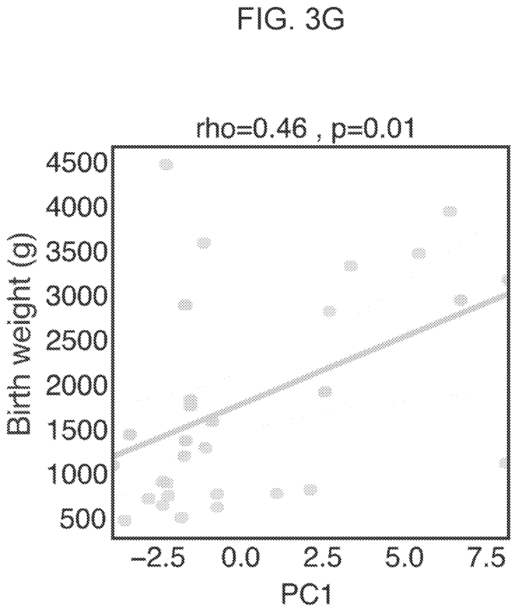

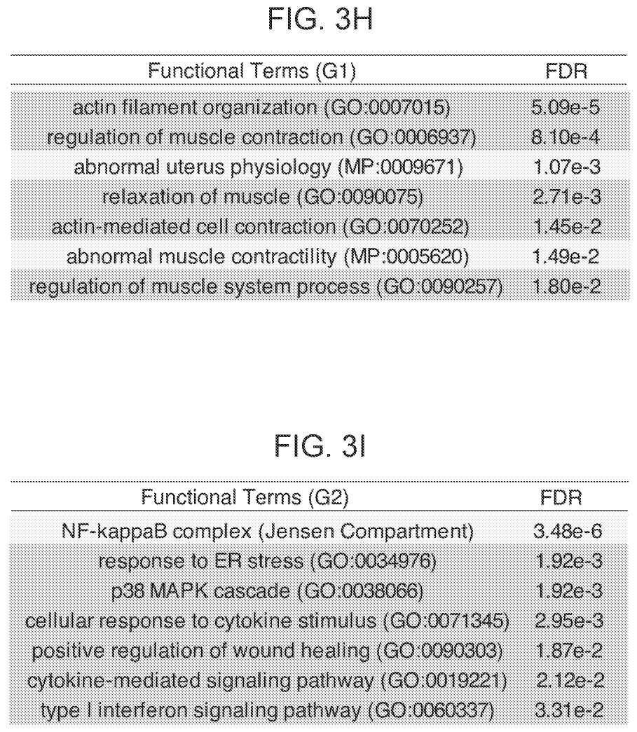

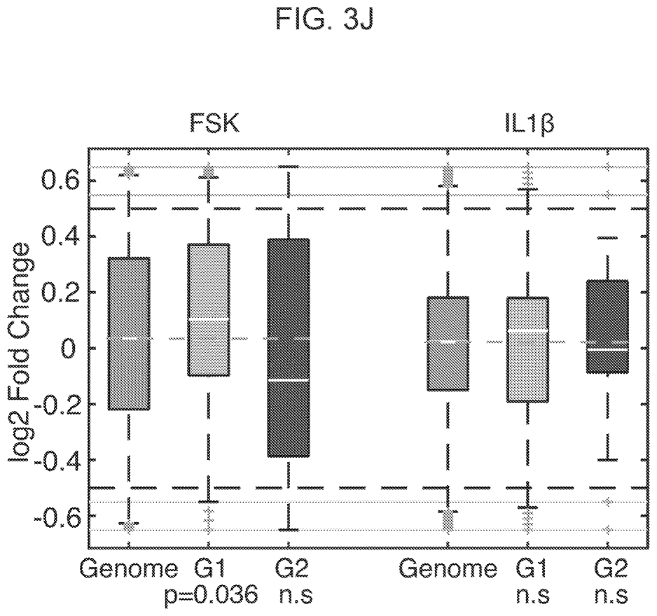

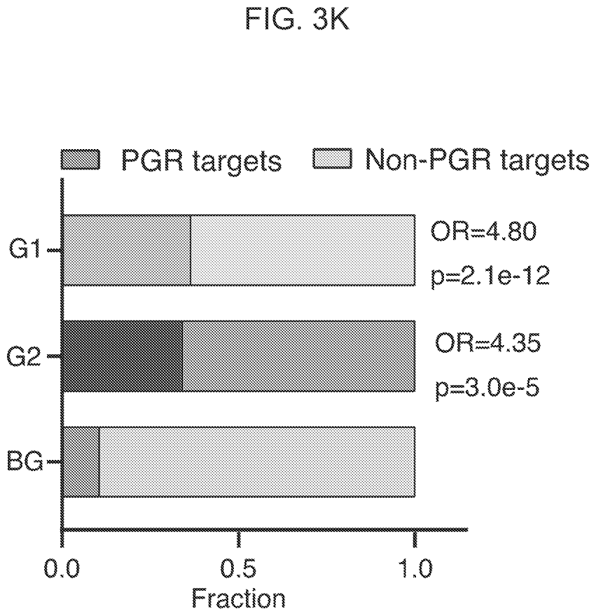

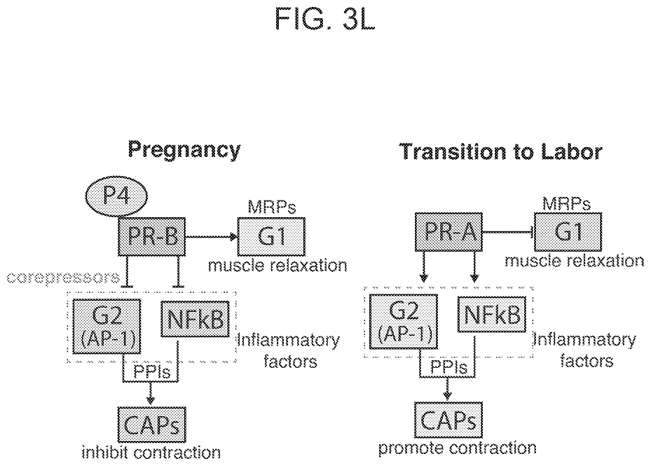

We compared the identified genes for their expression in the myometrium in the non-pregnant (Wu et al., 2020), non-laboring term pregnant (Wu et al., 2020) and labor onset conditions (Chan et al., 2014). We observed that the identified genes displayed a significant increase in their expression in the non-laboring term pregnant relative to the non-pregnant myometrium (P=1.47e4, Wilcoxon rank-sum test, FIG. 3A). However, when analyzing RNA-Seq data in myometrium at the labor onset (Chan et al., 2014), we observed that these genes displayed an overall marked down-regulation (P=8.76e-8, Wilcoxon rank-sum test, FIG. 3B) at the transition from quiescence to active labor. These observations collectively demonstrated the dynamics of their gene expression at key parturition stages. In addition to studying the global gene expression profiles, we then performed gene-wise differential expression test (Methods and Materials) and individually detected 1,268 and 1,037 up- and down-regulated protein-coding genes, respectively, upon labor onset (FDR≤0.01), including 99 among the 315 genes identified by our Bayesian framework (Table 3). Note that the detection of 31.03% of the identified genes (99/315) represented a significant enrichment for the differentially expressed genes, which was not expected by chance (P=1.40e-17, Fisher's exact test). While the 315 identified genes are likely associated with preterm labor by their genomic variants affecting myometrial physiologies (FIG. 2), to derive direct mechanistic insights, we elected to focus our in-depth analysis on these 99 genes given their strongest expression dynamics upon labor onset.

Analyzing expression profiles of the 99 genes revealed two cluster structures, where 69 genes formed an expression group (G1, FIG. 3C) displaying down-regulation upon labor onset, and 30 genes formed the up-regulated gene expression group (G2, FIG. 3C). Compared with the 1,268 up- and 1,037 down-regulated genes at the labor onset (at the same threshold FDR≤0.01, described above) across the myometrial transcriptome, the size of Group I genes outnumbering Group II was not expected by chance (P=3.43e-7, Fisher's exact test), confirming the specificity of the genes identified by our Bayesian model, not following background transcriptome distribution of the differentially expressed genes. Thus, the disproportionally enriched Group I genes likely explain the overall down-regulation of the BEAR genes in FIG. 3B.

We replicated our analysis leveraging an independent transcriptome dataset (Ackerman et al., 2021) from term myometrium samples before (N=5) and during labor (N=5), and confirmed the fact that expression alterations of Group I and II genes indeed hallmarked labor onset (FIG. 3D), where the expression dynamics independently replicated our observation in FIG. 3C. In addition to myometrium transcriptomes, the original dataset also included clinical measurements upon delivery (e.g. cervical diameters, uterine contractility and neonatal birth weight) across 31 individuals, which now have enabled us to directly associate expression of our identified genes with clinical physiologies. We analyzed expression of our identified Group 1 and 2 genes in the pregnant myometrium from each individual in this cohort (with clinically recorded physiological parameters upon delivery), and observed that expression variation of the identified genes (combining Group 1 and 2 genes) (represented by the first principal component) was strongly scaled by uterine contractility (rho=0.64, P=9.4e-5, Spearman's correlation), and was also significantly correlated with cervical dilation (rho=0.45, P=0.01, Spearman's correlation) and neonatal birth weight (rho=0.46, P=0.01, Spearman's correlation, FIGS. 3E-3G). These additional physiological measurements independently validated the clinical implications of our identified genes.

Performing functional enrichment analysis, we observed that the 69 Group I genes were highly enriched for functions associated with the regulation of muscle contraction (FDR=8.10e-4), more specifically with relaxation of muscle (FDR=2.71e-3, FIG. 3H). This explained their elevated expression prior to labor as well as their down-regulation upon delivery to promote muscle contractility. As expected, their mouse mutants displayed phenotypes involving abnormal uterus physiology (FDR=1.07e-3) and abnormal muscle contractility (FDR=1.49e-2, FIG. 3H). Because forskolin (FSK) promotes myometrial relaxation by stimulating cAMP synthesis (Yuan and Lopez Bernal, 2007), we examined RNA-Seq data from primary myometrial cell cultures treated with FSK (Stanfield et al., 2019a), and indeed observed significant up-regulation of Group-I genes upon FSK treatment (FIG. 3J). This observation indicated extensive responsiveness of Group-I genes to FSK in vitro, revealing their potential mechanistic involvement in myometrium relaxation before labor onset.

On the contrary, our functional enrichment analysis revealed that the 30 Group II genes were enriched for immune response and inflammatory factors (FIG. 3I). Note that inflammatory factors are known to promote muscle contractility during labor (Khanjani et al., 2011; Mendelson et al., 2019), explaining the down-regulation of Group II genes before labor and their activation during labor. We examined primary myometrial cell cultures treated with IL-1B to mimic tissue-level inflammation at the labor onset (Stanfield et al., 2019a). However, the inflammatory Group II genes were overall not responsive to the IL-1B treatment (P=0.68, Wilcoxson rank-sum test, FIG. 3J), suggesting that the identified genes were not involved in the IL-1B-mediated pathways. In fact, Group-II genes included a key inflammatory activators AP-1 (with its subunits JUN and FOS), which play a central role in regulating smooth muscle contractility proteins at labor onset (Mendelson et al., 2019). We also observed that Group-II genes were highly enriched for NF-κB interacting proteins (odds ratio, OR=21.72, and the adjusted P=3.48e-6, FIG. 3J, see Methods and Materials), thereby revealing the convergence of Group-II genes onto the AP1/NF-kB regulatory pathway(s) rather than the IL-1ß-mediated pathway(s).

In the literature, it is known that during pregnancy (in the quiescent state), the progesterone (P4)-liganded progesterone receptor (PR, isoform B, PR-B) maintains uterine quiescence by suppressing inflammatory factors (e.g., NF—KB and AP-1) and contraction-associated proteins (CAPs, such as CX43, OXTR, COX-2. In the meantime, PR-B also activates genes that promote muscle relaxation (e.g., PLCL1/2 (Peavey et al., 2021)). The opposing modes of action by PR are achieved through its interactions with different sets of co-factors (Mendelson et al., 2019). We examined PR ChIP-Seq data in the non-laboring term myometrial samples (Wu et al., 2020), and observed that both Groups I and II genes were significantly enriched for PGR transcriptional targets relative to the genome background (ORs >4, P<3.0e-5, Fisher's exact test, FIG. 3K). We therefore proposed a model illustrating how the P4-liganded PR exerts its regulatory effects on

Group-1 and 2 genes to maintain uterine quiescence before labor onset (FIG. 3L). However, during labor, the binding between progesterone (P4) and its receptor (PR) is compromised due to the PR isoform switch replacing the isoform B with its isoform A, a truncated and unliganded protein with significantly reduced transcriptional activity (Nadeem et al., 2016). This immediately results in suppression of muscle relaxation genes and activation of inflammatory factors (AP-1/NF-kB), leading to elevated smooth muscle contractility during labor (FIG. 3L). Taken together, our functional analysis indicated that the agnostically identified genes by our Bayesian model were in fact convergent onto the PR-mediated regulatory pathways: the coordinated expression alterations of Group-I and II genes navigate the quiescent myometrium into a contractile state hallmarking labor onset.

### Mutation Load in the Identified Genes Predict the Effectiveness of Progesterone Treatment for Recurrent Preterm Labor

We tested whether we could leverage these newly identified genes to foster the development of therapeutic strategies for preterm birth. To date, the only available medication for preterm birth is progestin prophylaxis, such as Makena® (17-hydroxyprogesterone caproate, 17-OHPC, injection), which received accelerated FDA approval in 2011 for reducing risk of preterm birth among the high-risk population (women with previous history). However, progestin therapy is associated with heterogenous results, with several clinical trials showing inconsistent effectiveness (Blackwell et al., 2020; Group, 2021; Meis et al., 2003). We posited that such heterogeneous observations likely resulted from genetic heterogeneity in the personal genomes. In other words, individuals carrying excessive mutations affecting the key molecular components in responding to the treatment would be less likely to be responders compared with those without significant mutational effects. We next investigated whether genomic mutations in the identified 99 genes could differentiate “responders” from “non-responders” to 17-OHPC treatment where response is defined as not delivering prematurely for those with previous history of recurrent preterm labor.

We performed a longitudinal patient recruitment in Alabama. The study involved 48 study participants who had previous history of spontaneous preterm labor (Supplementary Table 5 for patient information). All women admitted to the study were with singleton pregnancy and received weekly intramuscular injections of Makena 17-OHPC 250 mg beginning at 16 weeks until 36 weeks of gestation. Among the 48 study participants, 28 were considered responders to the treatment and delivered at term, whereas the remaining 20 were considered non-responders and had spontaneous preterm birth after the treatment. We acknowledged the possibility that those considered to be responders might include individuals who delivered at term owing to other reasons and not affected by the treatment. However, if we could observe genetic factors indeed differentiating the two groups, especially those involved in progesterone signaling, the observation then should be explained by progestin treatment.

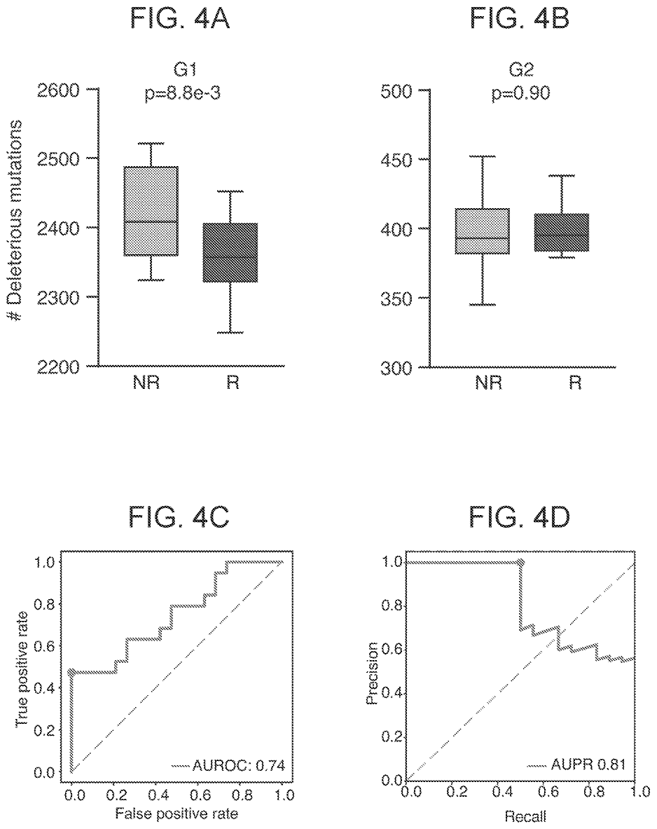

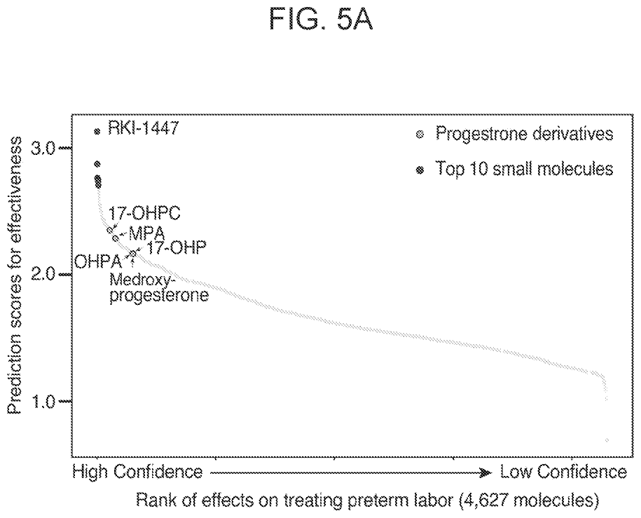

We performed whole genome sequencing (30×) and called on average 4.9 million genomic variants for each of the 48 women. QC analysis confirmed high-quality of the sequenced reads. Note that all genome sequencing procedures (DNA extraction, library prep, Hi-Seq sequencing, and QC, etc.) were blinded to the responder or nonresponder status. Because the vast majority of genomic mutations was localized in the noncoding genome, we leveraged our DEEP+ system (FIGS. 1A and 1B) to quantify the allelic effect at each genomic locus on perturbing chromatin accessibility in the non-laboring term-pregnant myometrium (the DEEP+ score). We considered deleterious regulatory mutations as those receiving extreme DEEP+ scores among the upper 5th percentile across the genome, regardless of whether they were common or rare variants. The called genomic variants from all the study participants were mapped onto the Group I (myometrial relaxation) and Group II (inflammatory response) genes identified in this study (FIG. 3C). Comparing the responders against the non-responders to progesterone therapy, we observed that Group I genes displayed a significant enrichment for deleterious regulatory mutations among the non-responders relative to the responders (P=9.9e-3, FIG. 6B); however, such a difference was absent from Group II genes (P=0.30, FIG. 6C). We performed additional control experiments to confirm the enrichment of deleterious mutations specific to Group I genes among the non-responders: (1) without considering mutational deleteriousness (DEEP+ scores), we considered the total number of genomic mutations, which however did not display significant difference between responders and non-responders in both Groups I and II genes (P>0.05, Wilcoxon rank-sum test). This comparison confirmed the efficacy of the DEEP+ system in distinguishing consequential mutations from the genome background; (2) because Group I genes displayed down-regulation at the labor onset (FIG. 3C), we performed the same comparison on all the down-regulated genes in the myometrium at the labor onset, and again we did not observe the mutational enrichment in these control genes among the non-responders (P=0.51, Wilcoxon rank-sum test. This comparison precluded the possibility that the mutational signature for Group I genes results primarily from down-regulation upon labor onset; (3) to confirm the specificity of Group I genes in differentiating responders from non-responders, we performed a negative control experiment, where we compiled a list of high-confidence genes in the neurodevelopmental program (Wang et al., 2020b) (Methods and Materials), without obvious functional associations with human pregnancy and parturition. This negative-control gene set was unable to identify the responder group from the non-responder group (P=0.26, Wilcoxon rank-sum test); (4) to confirm the observation was not affected by population demographics among the 48 study participants, we performed principal component analysis (PCA) to group patient ancestries based on their genome-wide mutation profiles, and identified a cluster of 36 patients with a strong African ancestry (FIG. 6A). Performing the same analysis on these 36 study participants, we observed a similar significant enrichment of deleterious mutations in Group I genes among the non-responders relative to responders (P=8.8e-3, Wilcoxon rank-sum test, FIG. 4A), and the enrichment was absent from Group II genes (P=0.90, Wilcoxon rank-sum test, FIG. 4B). Overall, because Group I genes were associated with muscle relaxation functions (FIGS. 3H and 3L) our observations thus demonstrated a positive association between mutational ablation of the muscle relaxation genes and the effectiveness of progesterone for recurrent preterm birth. As such, previous observation of heterogeneous clinical outcomes of progestin treatment might be explained, at least in part, by mutational diversity in patient populations that affect the myometrial muscle contraction/relaxation genes.

At the personal genome level, we calculated the mutation loads affecting the Group I genes in each patient, which demonstrated satisfactory predictive power for predicting clinical outcomes: increased mutation load predicts high likelihood of non-responsiveness (positive samples) with AUC=0.76 (FIG. 6D). We repeated the same analysis on the 36 individuals from the same population of African ancestry (described above, FIG. 6A), and obtained AUC=0.74 (FIG. 4C) for predicting non-responders to the treatment. We also computed AUPR (area under the precision recall curve)=0.81 (FIG. 4D), again confirming the predictability of the treatment non-responsiveness. Based on the existing data, the optimal threshold for deploying this model to screen patients was highlighted in FIGS. 4C, 4D, corresponding to an ~0% of false positive rate (~100% specificity) and ~50% of true positive rate (~50% sensitivity). The near perfect specificity implies that the model can confidently identify non-responders as long as the Group-I genes (regulating muscle relaxation) in personal genomes displayed excessive deleterious mutations. On the other hand, the ~50% sensitivity stems from the observation that individuals without significantly mutated Group-I genes could still be non-responders to the treatment. This could be explained by non-genetic factors or possibly by genetic factors not yet captured by this analysis. Nevertheless, we conclude that mutational ablation of the Group-I muscle genes is a sufficient but not necessary condition for the non-responsiveness of progesterone treatment. Therefore, based on our data, individuals with excessive deleterious mutations ablating the Group-I genes would very likely be non-responders and would not be advised to receive the treatment. On the contrary, given ~50% sensitivity in our analysis, individuals without significantly affected Group-I genes could benefit from the progesterone treatment. Taken together, these results revealed the importance of developing personalized strategies for treating recurrent preterm birth based on personal genomes, and our analytical framework could be deployed as a clinical tool to differentiate treatment responders from non-responders.

### Drug Discovery for Treating Spontaneous Preterm Labor

Progesterone supplementation is the only medication available to date for prevention spontaneous preterm birth. Identifying novel drug candidates modulating activities of the newly identified genes in this study could provide new opportunities for expanding therapeutic strategies. Conventional practice on drug discovery and repurposing has focused on identifying small molecules targeting single or very few proteins. However, increasing evidence has now shown that treatment effectiveness by drugs should be understood at a systems level. The efficacy of a small molecule, to a large extent, derives not from its capacity to directly target individual disease-associated proteins, but by its indirect and global impact on pathways disrupted in a given disease. As such, drug discovery on biological networks has achieved remarkable success (Barabasi et al., 2011; Cheng et al., 2019; Guney et al., 2016; Morselli Gysi et al., 2021; Ruiz et al., 2021). We followed this rationale to screen small molecules on a large-scale biological network. We built a multiscale network encompassing 18,757 human proteins, their mutual physical interactions and functional associations, as well as their known interactions with small molecules (Methods and Materials). We screened a comprehensive library of 4,293 FDA approved drugs, clinical trial drugs, and pre-clinical tool compounds from the Broad Drug Repurposing Hub (BDRH) (Corsello et al., 2017). Direct target proteins of the drugs were also retrieved from this BDRH resource for constructing the multiscale network. We adopted the latest network-based drug discovery algorithm, which models stochastic information flow on the network from a given drug to all possible proteins, and assigns each drug a score (i.e., likelihood of treatment) quantifying the global impact of the molecule on the network proteins in a given disease as functional modules. Therefore, a drug might not directly target known disease genes, but could propagate its impact on network to remotely connected proteins constituting disease-associated pathways, leading to effective treatment (Ruiz et al., 2021).

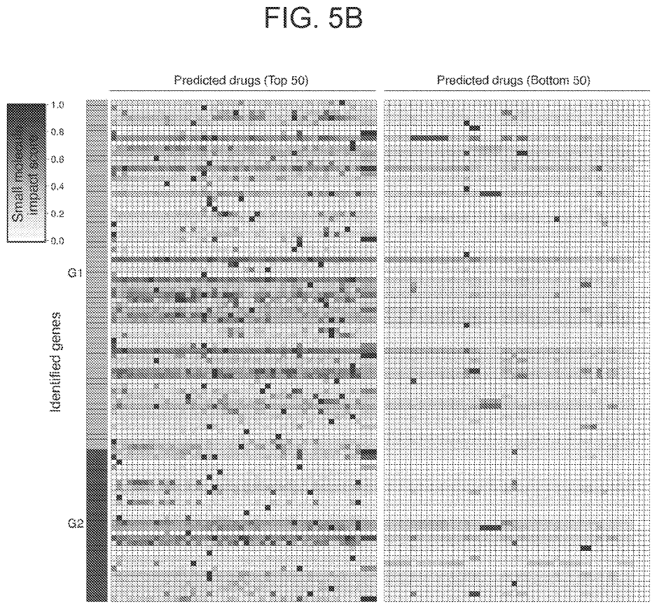

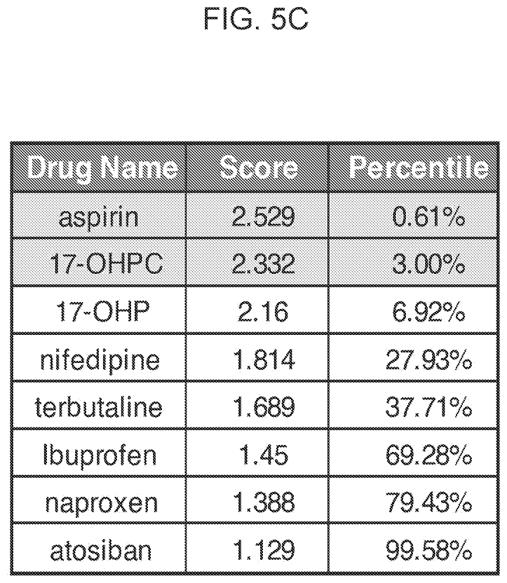

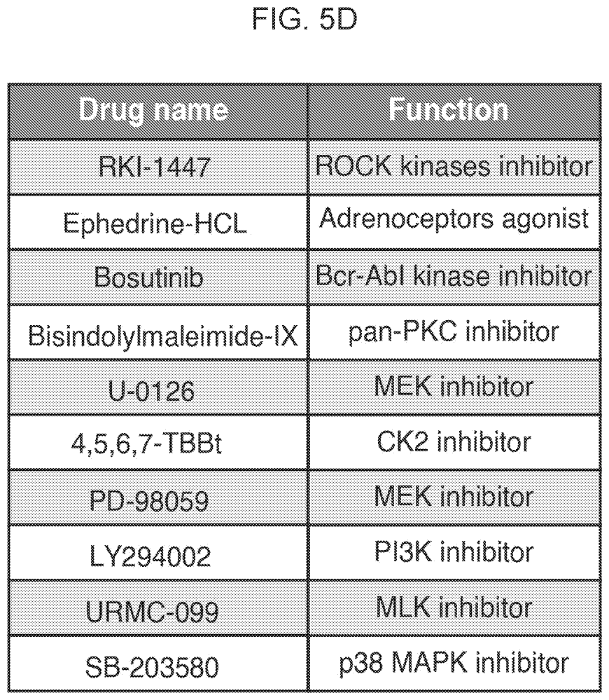

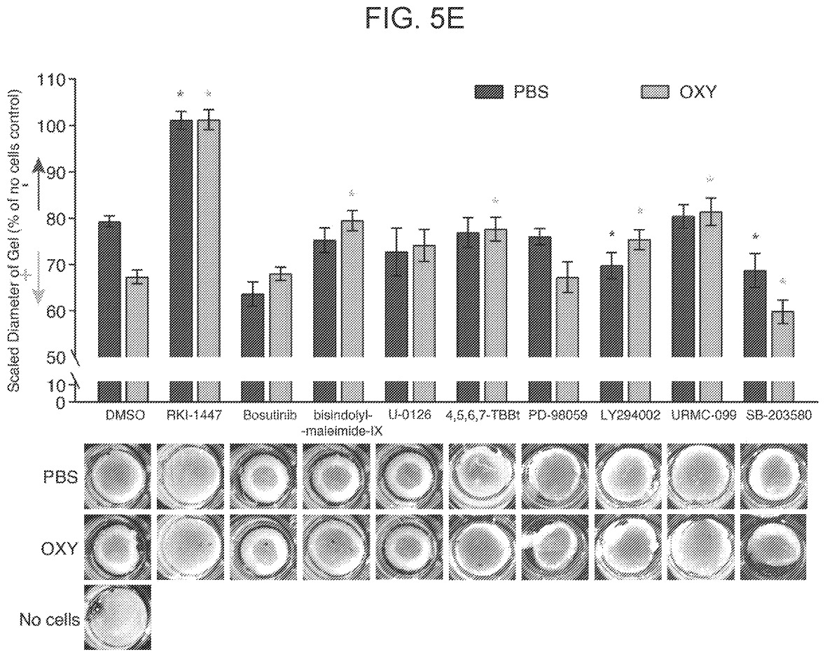

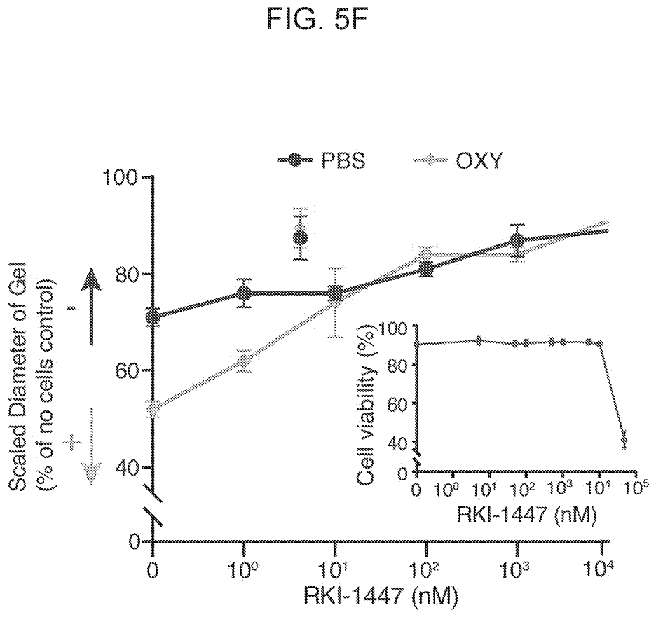

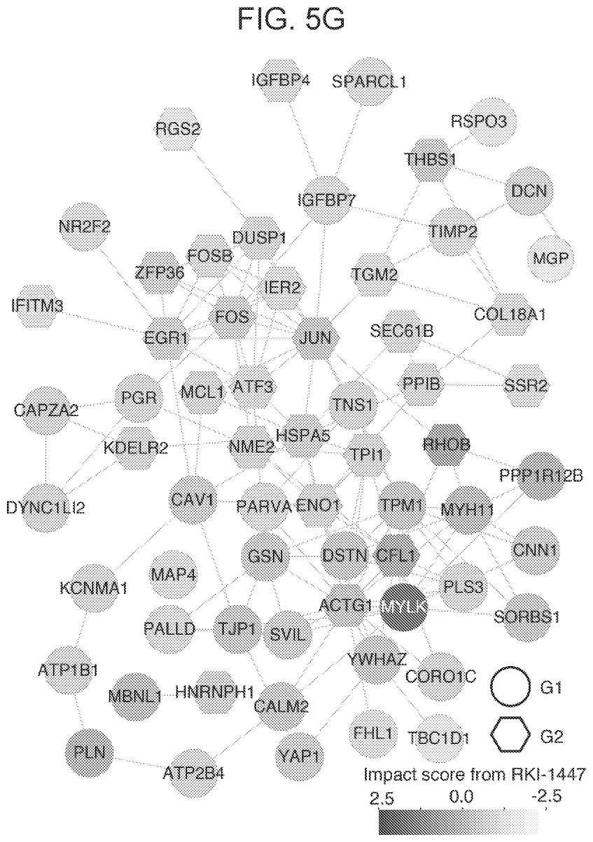

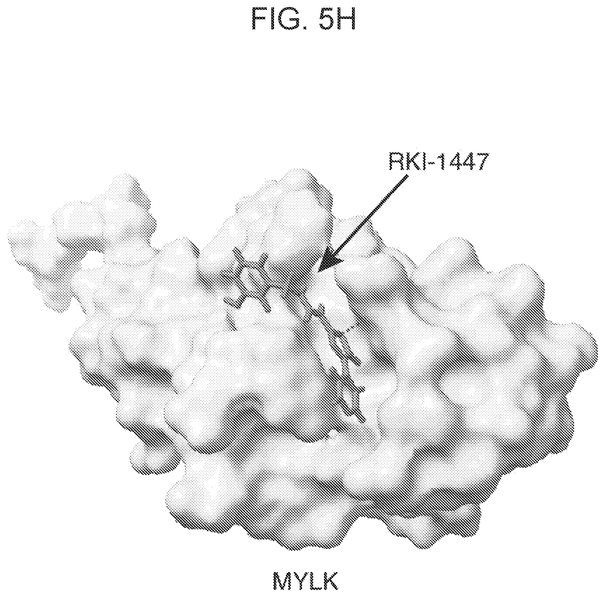

We implemented this algorithm (Ruiz et al., 2021) to screen each of the 4,293 BRDH small molecules, and ranked their impact scores on the 99 genes in spontaneous preterm birth (as a whole functional module on the network). The score distribution is shown in FIG. 5A, where we observed that the known treatment by 17-hydroxyprogesterone caproate (17-OHPC) was ranked among the upper 3% of all the drugs screened (128/4,293). In fact, progestin analogs as well as the endogenous progestogen steroid hormone (17-OHP) were all highly ranked (FIGS. 5A and 5C). We tested the model on small molecules that either were or had been used for sPTB prophylaxis or tocolysis, including NSAIDs, nifedipine, terbutaline, ritodrine, magnesium sulfate, and atosiban. Interestingly, in addition to the progestin analogs (e.g. 17-OHPC and 17-OHP), only aspirin was highly ranked by our scoring system (26/4,293, FIG. 5C), strongly indicating its therapeutic effect. This finding is supported by a recent large randomized, double-blinded, placebo-controlled trial (Hoffman et al., 2020), which showed that daily low-dose aspirin significantly reduced the risk of preterm birth (before 37 gestational weeks) by 11% and early preterm birth (before 34 gestational weeks) by 25% among women of their first pregnancies. In contrast, other previously used treatment did not receive high scores in our system (FIG. 5C), which likely explained their unsatisfactory performance in clinical use. Taken together, these comparisons suggested that our approach can indeed capture small molecules with therapeutic potential for spontaneous preterm birth, and closely examining the top ranked molecules might open an opportunity to uncover new therapeutic solutions.

When we compared the top 50 small molecules receiving the highest prediction scores against the bottom 50 small molecules with the lowest scores, we observed that the top 50 candidates were indeed more functionally related to the 99 preterm birth genes identified in this study, in contrast to a lack of connections on the network between these preterm birth genes and the small molecules receiving the lowest prediction scores (FIG. 5B). We next experimentally determine the effects of the top 10 highly ranked molecules for their potential therapeutic effects (FIG. 5D). Among the top 10 small molecules, 9 were prioritized for experimental characterization (FIG. 5D, Table 4). Ephedrine-hydrochloride was excluded as acquisition requires a physician's prescription.

The algorithm ranks the small molecules based on their functional relatedness with the identified genes in preterm birth but does not implicate the directionality of their modes of action (i.e., reduce or elongate the pregnancy duration). We performed experiments to determine the functional roles of the small molecules in regulating labor timing. Because labor onset is hallmarked by muscle contraction in the uterus, we asked whether these small molecules could alter smooth muscle contraction in the uterus. We performed collagen gel contraction assays on primary human uterine smooth muscle cells (HUtSMC) (Ying et al., 2015) to examine the modes of action of the top 9 candidate drugs from our prediction (FIG. 5D). For each drug, we tested its effect on modulating HUtSMC contractility in stimulated (by oxytocin to mimic labor-like phenotypes) and quiescent states by treating cells with oxytocin and vehicle, respectively. In either condition, we determined the pharmacological effects of our identified compounds on cellular contraction (each with at least 8 replicates). In the quiescent state (using PBS+DMSO as control, the blue bars in FIG. 5E), we observed that RKI-1447 strongly reduced the contraction (P=2.5e-5, FIG. 5E), whereas Bosutinib (P=2.0e-4), LY294002 (P=7.3e-3) and SB-203580 (P=3.6e-2) significantly induced myometrial cell contraction (FIG. 5E). In the contractile state stimulated by oxytocin (OXY, using OXY+DMSO as control, red bars in FIG. 5E), RKI-1447 (P=2.2e-9), bisindolylmaleimide-ix (P=1.4e-3), 4,5,6,7-tetrabromobenzotriazole (4,5,6,7-TBBt, P=1.4e-3), LY294002 (see Discussion, P=1.3e-2) or URMC-099 (P=5.5e-4) each attenuated myometrial contraction (FIG. 5E), whereas SB-203580 increased the contraction (FIG. 5E, P=1.8e-2). Overall, 5 out of the 9 compounds tested significantly decreased myometrial contraction, suggesting therapeutic potential to treat preterm labor. Combining all the observations, 7 among the 9 tested compounds affected myometrial cell contractility in either the quiescent or contractile state, validating the overall performance of our prediction for identifying small molecules that modulate uterine contractility. However, it is important to note that our observations do not preclude the potential therapeutic potential of other tested compounds to modulate uterine contractility. For example, despite the absence of signal from compounds U0126 and PD-98059, prior work demonstrated efficacy in a rat model of preterm labor with chronic treatment (Li et al., 2004). These observations provide additional support for the efficacy of our drug repurposing system.

We specifically highlight the top candidate from our prediction, which displayed the strongest signal on reducing muscle contraction in both quiescent and contractile states (FIG. 5E). We experimentally determined its dose-response relationship by varying its concentration from 0 to 10 μM (1e4 nM, FIG. 5F), and observed dose-dependent effects on myometrial smooth muscle relaxation in both quiescent (PBS) and contractile (OXY) states (FIG. 5F). We further confirmed that over a wide dose-range, RKI-1447 concentration had little effect on cell viability, with the emergence of cellular toxicity at a concentration of 50 μM (inset, FIG. 5F). These experimental data provided quantitative guidance on its potential clinical deployment. Overall, our human data are consistent with previous in vivo observations in rats where RKI-1447 attenuated uterine contractility (Domokos et al., 2017), providing further evidence of therapeutic potential to treat spontaneous preterm labor. To explore potential side effects on affecting smooth muscle cells in other organs, particularly its cardiotoxicity, we performed literature curation and confirmed that minimal effects from RKI-1447 (Ziegler et al., 2021). As a potent inhibitor of the Rho-kinases ROCKI/II, RKI-1447 has been mainly investigated for therapeutic effects on cancer (Dyberg et al., 2019; Li et al., 2020; Patel et al., 2012), glaucoma (Cush et al., 1995), and nonalcoholic fatty liver disease (Wang and Jiang, 2020), with less focus on putative effects on modulating uterine contractility. Interestingly, in our network-based drug discovery framework, RKI-1447 demonstrated strong functional relatedness with many muscle proteins, such as MYLK, MYH11, MBNL1, TPM1, PLN (FIG. 5G). The affected proteins also included SORBS1 associated with preterm birth as shown in FIG. 1F. Specifically, for the well-known muscle protein MYLK (the myosin light chain kinase), which received the strongest impact score of RKI-1447, we performed protein docking analysis and observed its binding affinity with RKI-1447 (FIG. 5H). These observations provided mechanistic basis for our drug discovery and experimental observations (FIGS. 5E and 5F).

### Discussion

More than 90% of loci in complex diseases are located in the non-coding genome (Boix et al., 2021; Corradin and Scacheri, 2014; Schaub et al., 2012), so we decided to decipher the regulatory genomic landscape in spontaneous preterm birth, the leading cause for neonatal morbidity and mortality. We developed DEEP+ to score the consequence of each nucleotide change across the genome in altering chromatin architecture in the non-laboring term myometrium, enabling genome-wide quantification of tissue-specific mutational effects (FIG. 1A). To narrow down deleterious mutations that contribute to spontaneous preterm birth, we further developed the BEAR probabilistic graphical model to integrate the tissue-specific epigenome, the GWAS risk profiles, as well as many other genomic resources (FIGS. 1F and 1G). Among ~13 million genomic variants analyzed across the genome, we identified 1,079 candidate loci in spontaneous preterm birth, mapped onto 315 protein-coding genes. These loci received extreme BEAR scores (account for upper 0.01% across all the genomic loci) and were strongly backed by multi-dimensional genomic evidence in our analysis. However, given the polygenic or even “omnigenic” nature of complex human diseases (Boyle et al., 2017), many more genomic loci might also contribute to the molecular etiologies of spontaneous preterm birth. In addition to identifying extreme signals, our model can be leveraged to rank the “omnigenic” signals across the genome.

In comparison with the widely used statistical association framework for disease genome analysis, our Bayesian model aims to characterize disease genomes from a different perspective. The widely used GWAS model infers disease association using allele frequency imbalance between case and control cohorts at every genomic locus. However, our analysis has enabled us to directly score mutational consequences at a single-base resolution across the genome and the scoring is specific to tissue(s) (which can be easily extended to cell types) that are most relevant to a given disease. Constructing a Bayesian model to integrate this tissue-specific epigenomic information with GWAS risk therefore generated novel findings that were strongly supported by independent experimental data by multi-omic profiling data in human clinical samples. We noted a recent publication which examined GWAS signals colocalized in epigenetically active regions during decidualization and suggested at-risk loci in preterm birth (Sakabe et al., 2020). However, signal colocalization does not imply mutational disruption, thereby requiring future experimental validation. It is also important to note that decidualization, the process of transforming mesenchymal stromal/stem cells to decidual stromal cells, occurs at early stages of pregnancy with clear significance in pregnancy establishment, but its role in the pathogenesis of preterm labor remains unclear. Therefore, in this work, we elected to investigate mutational effects in the non-laboring term-pregnant myometrial samples, which provided an appropriate context for studying spontaneous preterm birth. Because preterm labor is a syndrome with many causes (Romero et al., 2014), it is expected that our Bayesian and deep learning models using myometrium samples would not fully explain all clinical cases in our study cohort. Future studies utilizing other tissue types is warranted, especially considering the placental epigenome. It is important to note that our model development was impartial to tissue types, and the statistical learning model presented in this work can be easily applied to studying the contribution to preterm labor from any other tissue/cell types.

Our model identified 315 genes in spontaneous preterm birth, which displayed functional enrichment for regulating myometrial relaxation and activating inflammatory responses. In fact, the two functional categories distinguish pregnancy and the onset of labor, where tissue-level inflammation is associated with myometrial muscle contraction (Mendelson et al., 2019; Nadeem et al., 2016). The consistency with our current knowledge about spontaneous preterm labor provided independent support for our model performance. Specifically, for the identified genes regulating muscle functions (FIG. 2), we observed that excessive pathogenic mutations preferentially affected the proteins maintaining muscle relaxation during pregnancy, and their mutational disruption is expected to lead to preterm myometrial contraction. These proteins include known muscle-contraction inhibitors such as CALD1, as well as several other factors such as myosin proteins whose regulatory function for muscle contraction and relaxation is often dynamically regulated by the phosphorylation process (Sweeney, 1998). It is important to note that the identified muscle relaxation and inflammatory activation processes are not independent but are coordinated to initiate the natural labor onset, and in our study, we showed that the two gene groups were convergent onto the PGR-mediated regulatory pathway. Among the 315 genes, our in-depth analysis was focused on a subset that displayed strongest expression alterations at labor onset, suggesting their potential roles in regulating labor timing. As such, their highly scored noncoding regulatory variants by our model likely perturbed their expression, predisposing individuals to risk of preterm birth. Our integrative functional genomic analyses revealed the modes of action for the identified genes before and after labor onset (FIG. 3L); while the progesterone receptor plays a central role in driving the transition from pregnancy to parturition, the regulatory dynamics is also coordinated by many co-factors. In fact, when examining PGR binding sites in G1 and G2 genes, we also observed significant enrichment for TEAD and SRF binding motifs in G1 genes and AP-1 binding motif in G2 genes, respectively. Such co-occupancy signals suggested that PGR regulates G1 and G2 genes with different sets of cofactors, achieving activation of G1 genes for muscle relaxation and repression of G2 genes in inflammatory responses before labor. Particularly for G2 genes, in addition to their strong enrichment for inflammatory factors, this gene group also displayed a significant enrichment for response to ER stress (FDR=1.92e-3, FIG. 3H). Increasing ER stress helps maintain uterine quiescence and the stress level is reduced near term (Kyathanahalli et al., 2015; Suresh et al., 2013); therefore, in addition to inflammatory responses, G2 genes might also regulate labor timing through responding to ER stress. It is critical to emphasize that because all the identified genes in this study were based on our quantification of functional consequences of their associated noncoding regulatory mutations, this study indicates the etiological contribution to preterm labor from the noncoding genome, necessitating more in-depth characterization of the regulatory landscapes in future studies.

Insight into the discrete molecular determinants of spontaneous preterm labor provided a unique opportunity to develop therapeutic strategies for this condition. Our curiosity was initially triggered by the highly discordant observations on the treatment outcomes of progesterone therapy from several independent clinical trials (Blackwell et al., 2020; Meis et al., 2003; Stewart et al., 2021). The recent heated debate on withdrawing progesterone therapy proposed by the FDA (Chang et al., 2020; Greene et al., 2020) further motivated us to consider treatment responses at a personal level as opposed to our conventional practice at a population level. Our data now indicated that individuals with significantly mutated G-1 genes were almost perfectly predicted to be non-responders (positive samples) to the treatment (almost zero false positives), whereas our ~50% sensitivity in prediction resulted from the existence of false negatives (true positives that were predicted to be negatives), i.e. individuals without significantly mutated G-1 genes were still not responding to the treatment. This was anticipated given potential non-genetic factors underlying treatment responses. This observation on genetic heterogeneity likely explains the observed heterogeneity in clinical outcomes in previous clinical trials, but more importantly, it provides a more practical guidance on clinically administering the treatment at a personal genome level: for individuals without significant G-1 mutations, the chance to receive clinical benefit from this treatment is in fact substantial. From a mechanistic perspective, although the progesterone receptor regulates both muscle contraction and inflammatory genes at labor onset (FIG. 3L) (Wu et al., 2020), our study now revealed that it was in fact the muscle relaxation component that determined progesterone responses, where Group Il genes (the inflammatory factors) were not associated with clinical outcomes. Thereby, by targeting Group I genes, our mutation scoring model DEEP+ could be deployed as a screening tool to evaluate clinical benefits of administering the treatment. Taken together, this study calls for a precision-medicine framework for future clinical trials, where drug efficacy should be evaluated at an individual level taking into account personal genomes and lifestyles, compared with our existing practice based on population averages.

In the past decades, only a single medication (progesterone therapy) was widely used for treating preterm labor. Identifying genes in spontaneous preterm labor has now enabled us to extend our view from progesterone to other promising strategies. We exhaustively and agnostically screened 4,293 small molecules for their potential effects on regulating labor timing, and indeed observed that many more compounds were highly scored than progesterone derivatives (FIG. 5A). We experimentally validated the top ranked compounds in our prediction: among 9 compounds tested, seven (RKI-1447, bisindolylmaleimide-ix, TBBt, LY294002, URMC099, Bosutinib, and SB-203580) altered myometrial contractility in either quiescent or contractile state, and five (RKI-1447, bisindolylmaleimide-ix, TBBt, LY294002, URMC-099) attenuated oxytocin-induced myometrial contractility, demonstrating therapeutic potential to treat preterm labor. Notably, bisindolylmaleimide-IX (a pan-PKC inhibitor), 4,5,6,7-tetrabromobenzotriazole (a CK2 inhibitor), LY294002 (a PI3K inhibitor) and URMC-099 are all pre-clinical drugs, which on average reduced oxytocin-induced myometrial contractility by 15.73% (FIG. 5D). We particularly noted that LY294002 blocked myometrial contraction induced by endothelin-1 (ET-1) in a previous study (Di Liberto et al., 2003). Our assay now suggested its effect on oxytocin-induced contraction as well. Intriguingly, in a quiescent state without oxytocin induction, this compound alone displayed a significant effect on increasing myometrial contractility (FIG. 5D), thereby suggesting its potential pharmacological interaction with oxytocin. Among all the drugs tested, RKI-1447 was the only drug to prevent myometrial cell contractility in the presence and absence of oxytocin. Moreover, our analysis associated this Rho kinase inhibitor with molecules involved in modulating the contractile state of smooth muscle cells, thereby underscoring the importance of RKI-1447 mechanism of action in regulating uterine contractility.

Limitation of the Study. (1) This study maps genomic mutations in preterm birth patient genomes onto the reference myometrium epigenome at term to identify at-risk loci in preterm birth. Therefore, the identified variants as well as the associated genes are expected to explain genetic risk of a subset of patients with tissue of origin in the myometrium. Given multiple causes leading to preterm birth, future analysis is needed to investigate molecular etiologies in many other tissues, especially in the placenta. The developed model in this analysis can be readily extended to integrating patient genomes with epigenomes from any other tissues. (2) We used the epigenome in the term myometrium as a reference to study mutational effects on dysregulating gene expression associated with regulating labor timing. This practice was based on the share clinical characteristics between term and preterm labor (Mendelson et al., 2019). However, it is possible that genomic mutations predispose individuals to preterm labor by perturbing regulatory elements only active in early gestation, which therefore would not be captured in our screen. Future work would be needed to construct a time-course epigenome map in the myometrium across gestational weeks, which would allow us to identify the most vulnerable time window underlying the development of this clinical condition. (3) We leveraged the newly identified genes to guide our agnostic drug discovery and we prioritized the top 9 molecules for in vitro validation. These in vitro data were overall concordant with our prediction scores. Therefore, future in vivo analysis is needed to refine our drug screening results. Although we only tested the top 9 drugs in this analysis, many other highly ranked small molecules could still be effective on treating preterm labor, best exemplified by aspirin (ranked at 26/4,293) discussed in FIG. 5C. Therefore, future high-content screening approaches is highly desired to verify drug effectiveness immediately after AI-guided drug discovery.

### Method

### ATAC-Seq Library Construction and Sequencing

ATAC-seq was performed with Tagment DNA TDE1 Enzyme and Buffer Kits (Illumina, San Diego, USA) as per instruction. Briefly, ~50,000 nuclei were isolated from 5 primary frozen myometrium samples following the Omni-ATAC protocol (28846090) after homogenization, centrifugation and permeabilization. The collected nuclei were then incubated with Tn5 transposase in 1×TD buffer. The purified DNA were then PCR amplified with optimal cycles to construct ATAC-seq libraries, which were subsequently sequenced on NextSeq 500 (Illumina) to yield ~50M reads per samples.

Calling ATAC-Seq Peaks from the Myometrium

ATAC-seq data from the non-laboring and term pregnant myometrium (GSE137552) (Wu et al., 2020) were aligned to the human genome bowtie2 (v2.3.5.1) (Langmead & Salzberg, 2012) with default settings. Peak calls were implemented using MACS2 (v2.1.4) (Zhang et al., 2008) with default settings. Reads with MAPQ >30 and mapped in proper pair were retained for further analysis. Reads with duplicates were marked by “sambamba markdup” (Tarasov et al., 2015). Then we used MACS2 (v2.1.4) (Zhang et al., 2008) to call peaks with parameters -q 0.1 -B- SPMR --nomodel --shift 75 --extsize 150 --keep-dup-all. We used “bedtools intersect” (Quinlan and Hall, 2010) to identify peaks commonly identified in all replicates. The HOMER package (annotatePeaks.pl) (Heinz et al., 2010) was used to annotate the peaks and calculate motif enrichment. The human genome was based on hg19 throughout the manuscript.

### The DEEP+ Model

We extended our previous DEEP model (Wang and Li, 2020) to the DEEP+ model by replacing the convolutional neural network architecture with a residual deep neural network (He et al., 2016). The residual network introduces shortcuts to jump over layers in terms of avoiding the potential gradient explosion during the model training with an increase in the number of network layers. In this study, DEEP+ outperformed our original DEEP model on the myometrium ATACSeq data. In brief, DEEP+ included four residual blocks, and each block consisted of one base unit with three convolutional layers. The shortcuts linked information flow between different blocks. All convolutional layers were activated by ReLU after batch normalization. We set a 20% dropout rate between the neighboring residual blocks to minimize the risk of overfitting. We trained the DEEP+ model using the ATAC-Seq data in the primary human myometrium tissues (non-laboring term pregnant, three biological replicates, GSE137552) (Wu et al., 2020). We reanalyzed the published ATAC-Seq data to call narrow peaks in the BED format. We split the peak regions into multiple 200 bp windows and extended 900 bp flanking sequences at both upstream and downstream as sequence context for training input (Zhou et al., 2018). In our blind tests, we followed the protocol in previous work (Wang and Li, 2020), where every time we held out ATAC-Seq data from one chromosome for independent performance evaluation. For the remaining ATAC peaks, we randomly selected 5% for the purpose of model validation and optimization, and rest 95% peaks for model training. We further evaluated the performance of the established model with common ATAC peaks identified in 5 independent myometrium samples. The performance of models was assessed by AUROC (area under the receiver operating characteristic curve) and AUPR (area under the precision-recall curve). To quantify the mutational consequence for each variant, we implemented our DEEP+ model to compute the absolute difference between the predicted chromatin openness scores from the genomic sequence centered on two alleles of a given mutation.

eQTL Analysis

The HC and LC eQTLs were defined based on the CAVIAR fine-mapping of eQTLs that are downloaded from GTEx Analysis V8 (dbGaP Accession phs000424.v8.p2) (gtexportal.org/home/datasets)

### Bayesian Estimation of the Altered Regulation

BEAR (Bayesian Estimation of Altered Regulation) is a hierarchical Bayesian model genetic loci in a given disease by aggregating evidence from multiple dimensions, including: (1) the impact on tissue-specific epigenomes for each noncoding genomic variants; (2) the GWAS effect size for each genomic variant; (3) the tolerance to dosage alteration for a gene associated with a given variant; (4) the molecular activity of genes of interest in a given tissue/cell type. BEAR integrates all the information and derives a posterior probability for each genomic variant for its implication in a disease conditioned on all the above data. Throughout this study, unless otherwise mentioned, we associated a variant to a gene with the nearest TSS by Homer v4.11 (http://homer.ucsd.edu/homer/index.html). Details of model construction and inference can be found in Supplementary Notes.

### Differential Gene Expression Analysis

Transcriptomic data in the human myometrium from individuals at term in labor (TIL) and not in labor (TNL) were obtained from a previous work (GSE50599) (Chan et al., 2014). Differentially expressed genes were identified using DESeq2 with default settings (Love et al., 2014). We considered statistical significance when the adjusted p values were less than 0.01.

We downloaded normalized FPKM gene expression matrix of RNA-seq data (GSE134896 and GSE163773) (Ackerman et al., 2021; Stanfield et al., 2019a) from GEO for primary myometrial cell cultures following treatment with Forskolin and Interleukin-1B, and for primary myometrium samples harvested from 31 women at labor. We performed PCA on expression matrix of selected genes for all 31 samples and picked the first principal component to calculate the Spearman coefficient with selected clinical features. All p values were derived from Wilcoxon rank-sum test.

### Gene Ontology Analysis

Gene ontology enrichment of obtained gene lists were carried out by g: Profiler (Raudvere et al., 2019) followed by GOMCL clustering (Wang et al., 2020a). We first applied GOMCL.py with parameters -gosize 3500 -gotype BP CC MP -I 1.5 -Ct 0.5 -SI OC -Sig 0.01 to cluster GO terms. Then the clustering results were further separated into sub-clusters by GOMCL-sub.py with parameters -C 1 -Ct 0.6 -gosize 2000 -I 1.8. Cytoscape (Otasek et al., 2019) was used for visualization. For the purpose of control, we also studied gene ontology enrichment for randomly sampled genes with matched size and gene expression level with our identified genes. For enrichment analysis of non-GO terms, the odd ratios and p values were obtained from Enrichr (Kuleshov et al., 2016).

### Allelic Expression Analysis

Expression data of three primary myometrium at term (GSE137551) (Wu et al., 2020) were aligned to hg19 with STAR (Dobin et al., 2013). Then we counted the expressed alleles identified in curated European individuals from phase 3 1000 Genomes (Genomes Project et al., 2015) with ASEReadCounter in GATK (McKenna et al., 2010). For specific risk alleles, we calculated the expression ratio of all correlated alleles within the LD blocks when setting R²>0.6 with LDlink (Machiela and Chanock, 2015) based on data from Europeans in 1000 Genome (Genomes Project et al., 2015).

### Patient Recruitment and Whole Genome Sequencing

We performed longitudinal recruitment of 48 women in Alabama who had previous history of spontaneous preterm labor. All women admitted to the study were with singleton pregnancy and received weekly intramuscular injections of Makena OHP 250 mg beginning at 16 weeks until 36 weeks of gestation. This study was approved by Stanford Institutional Review Board (IRB #21956). We harvested the peripheral blood from the individuals and performed whole genome sequencing at an average coverage at 30×. The sequencing experiments were blinded to clinical outcomes.

Variant calls were made by following the Best Practice procedure recommended by GATK (Van der Auwera et al., 2013). We performed independent quality control analyses to ensure high quality of the called variants. We utilized VCFtools (http://vcftools.sourceforge.net) to compute the distribution of Ts/Tv ratios across all analyzed individuals. We then assessed the population ancestries on these called variants among the 48 individuals by principal component analysis (PCA) from plink (v1.90b6.17) (Purcell et al., 2007). For each personal genome, we counted the numbers of deleterious mutations (the top 5% of DEEP+ score across the entire genomes in this cohort) that mapped to genes of interest.

Drug Discovery with the Multiscale Interactome

We modified the multiscale interactome network (Ruiz et al., 2021) with protein-protein interaction data from STRING (string-db.org/), and the drug-protein interaction from Broad Drug Repurposing Hub (BDRH) (Corsello et al., 2017). This multiscale interactome captures 11,444 drug-protein edges between 18,757 protein and 4,293 drug nodes. We designed a disease node representing spontaneous preterm birth on the network, which is connected with 99 proteins identified in this study. The random walk-based algorithm was implemented with optimal edges weights (Ruiz et al., 2021) to derive the diffusion profiles on all protein nodes from each drug or disease node. The similarity of diffusion profiles between all drugs and sPTB node was quantified and ranked by Jenson-Shannon divergence, where top 10 drug candidates were selected for experimental validation.

### Human Uterine SMC Contraction Assay

Primary Human Uterine Smooth Muscle Cells (HUISMC) were purchased from PromoCells (Cat #C-12575, Fisher Scientific, MA). Collagen gel contraction assays were performed following (Kita et al., 2008; Ying et al., 2015) with modifications. Briefly, 150 ul of collagen (Cat #5074, SigmaAldrich) was added to each well in a 48-well plate. 80,000 HUtSMC suspension in 300 ul Smooth Muscle Cell Growth Basal Medium (Lonza #CC-3181) were seeded to each well after collagen polymerization for an hour. The cells were then treated with either testing compounds or DMSO in the presence of 100 nM oxytocin or PBS control. We carefully detached the collagen from the wells by tips after 1 hour incubation at 37° C., and fixed the cells with 4% paraformaldehyde in PBS for 30 minutes after 18 hours to ensure the equivalent culture durations for all groups before imaging. We captured bright-field images with microscope systems (AmScope, Irvine, CA), and measured the gel diameters in the captured images by ImageJ where the fold changes to no cell control were taken for statistical analysis. At least three biological replicates were performed for each compound, and technically triplicates were included in each biological replicate. Information about small molecules in the study could be found in Table 4.

### Docking Analysis

We implemented swissDock (Grosdidier et al., 2011) to predict the interactions between RKI-1447 and MYLK as well as their binding affinity. The PDB structure for MYLK is 2 yr3 (Model 1).

### REFERENCES

Ackerman, W. E. t., Buhimschi, C. S., Snedden, A., Summerfield, T. L., Zhao, G., and Buhimschi, I. A. (2021). Molecular signatures of labor and nonlabor myometrium with parsimonious classification from 2 calcium transporter genes. JCI Insight 6.
Amini, P., Wilson, R., Wang, J., Tan, H., Yi, L., Koeblitz, W. K., Stanfield, Z., Romani, A. M. P., Malemud, C. J., and Mesiano, S. (2019). Progesterone and cAMP synergize to inhibit responsiveness of myometrial cells to pro-inflammatory/pro-labor stimuli. Mol Cell Endocrinol 479, 1-11.
Andersson, R., Gebhard, C., Miguel-Escalada, I., Hoof, I., Bornholdt, J., Boyd, M., Chen, Y., Zhao, X., Schmidl, C., Suzuki, T., et al. (2014). An atlas of active enhancers across human cell types and tissues. Nature 507, 455-461.
Barabasi, A. L., Gulbahce, N., and Loscalzo, J. (2011). Network medicine: a network-based approach to human disease. Nat Rev Genet 12, 56-68.
Bethin, K. E., Nagai, Y., Sladek, R., Asada, M., Sadovsky, Y., Hudson, T. J., and Muglia, L. J. (2003). Microarray analysis of uterine gene expression in mouse and human pregnancy. Mol Endocrinol 17, 1454-1469.
Blackwell, S. C., Gyamfi-Bannerman, C., Biggio, J. R., Jr., Chauhan, S. P., Hughes, B. L., Louis, J. M., Manuck, T. A., Miller, H. S., Das, A. F., Saade, G. R., et al. (2020). 17-OHPC to Prevent Recurrent Preterm Birth in Singleton Gestations (PROLONG Study): A Multicenter, International, Randomized Double-Blind Trial. Am J Perinatol 37, 127-136.
Boix, C. A., James, B. T., Park, Y. P., Meuleman, W., and Kellis, M. (2021). Regulatory genomic circuitry of human disease loci by integrative epigenomics. Nature 590, 300-307.
Boyle, E. A., Li, Y. I., and Pritchard, J. K. (2017). An Expanded View of Complex Traits: From Polygenic to Omnigenic. Cell 169, 1177-1186.
Chan, Y. W., van den Berg, H. A., Moore, J. D., Quenby, S., and Blanks, A. M. (2014). Assessment of myometrial transcriptome changes associated with spontaneous human labour by high-throughput RNA-seq. Exp Physiol 99, 510-524.
Chang, C. Y., Nguyen, C. P., Wesley, B., Guo, J., Johnson, L. L., and Joffe, H. V. (2020). Withdrawing Approval of Makena-A Proposal from the FDA Center for Drug Evaluation and Research. N Engl J Med 383, e131.
Chen, X., Zhang, X., Li, W., Li, W., Wang, Y., Zhang, S., and Zhu, C. (2021). Iatrogenic vs. Spontaneous Preterm Birth: A Retrospective Study of Neonatal Outcome Among Very Preterm Infants. Front Neurol 12, 649749.
Cheng, F., Kovacs, I. A., and Barabasi, A. L. (2019). Network-based prediction of drug combinations. Nat Commun 10, 1197.
Consortium, G. T. (2020). The GTEx Consortium atlas of genetic regulatory effects across human tissues. Science 369, 1318-1330.
Corradin, O., and Scacheri, P. C. (2014). Enhancer variants: evaluating functions in common disease. Genome Med 6, 85. Corsello, S. M., Bittker, J. A., Liu, Z., Gould, J., McCarren, P., Hirschman, J. E., Johnston, S. E., Vrcic, A., Wong, B., Khan, M., et al. (2017). The Drug Repurposing Hub: a next-generation drug library and information resource. Nat Med 23, 405-408.
Cush, J. J., Splawski, J. B., Thomas, R., McFarlin, J. E., Schulze-Koops, H., Davis, L. S., Fujita, K., and Lipsky, P. E. (1995). Elevated interleukin-10 levels in patients with rheumatoid arthritis. Arthritis Rheum 38, 96-104.
Di Liberto, G., Dallot, E., Eude-Le Parco, I., Cabrol, D., Ferre, F., and Breuiller-Fouche, M. (2003). A critical role for PKC zeta in endothelin-1-induced uterine contractions at the end of pregnancy. Am J Physiol Cell Physiol 285, C599-607.
Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21.
Domokos, D., Ducza, E., Falkay, G., and Gaspar, R. (2017). Alteration in expressions of RhoA and Rho-kinases during pregnancy in rats: their roles in uterine contractions and onset of labour. J Physiol Pharmacol 68, 439-451.
Dyberg, C., Andonova, T., Olsen, T. K., Brodin, B., Kool, M., Kogner, P., Johnsen, J. I., and Wickstrom, M. (2019). Inhibition of Rho-Associated Kinase Suppresses Medulloblastoma Growth. Cancers (Basel) 12.
Ehn, N. L., Cooper, M. E., Orr, K., Shi, M., Johnson, M. K., Caprau, D., Dagle, J., Steffen, K., Johnson, K., Marazita, M. L., et al. (2007). Evaluation of fetal and maternal genetic variation in the progesterone receptor gene for contributions to preterm birth. Pediatr Res 62, 630-635.
Fabregat, A., Jupe, S., Matthews, L., Sidiropoulos, K., Gillespie, M., Garapati, P., Haw, R., Jassal, B., Korninger, F., May, B., et al. (2018). The Reactome Pathway Knowledgebase. Nucleic Acids Res 46, D649-D655.
Fuchs, A. R., Fuchs, F., Husslein, P., and Soloff, M. S. (1984). Oxytocin receptors in the human uterus during pregnancy and parturition. Am J Obstet Gynecol 150, 734-741.
Genomes Project, C., Auton, A., Brooks, L. D., Durbin, R. M., Garrison, E. P., Kang, H. M., Korbel, J. O., Marchini, J. L., Mccarthy, S., McVean, G. A., et al. (2015). A global reference for human genetic variation. Nature 526, 68-74.
Goldenberg, R. L., Culhane, J. F., lams, J. D., and Romero, R. (2008). Epidemiology and causes of preterm birth. Lancet 371, 75-84.
Greene, M. F., Klebanoff, M. A., and Harrington, D. (2020). Preterm Birth and 17OHP-Why the FDA Should Not Withdraw Approval. N Engl J Med 383, e130.
Grosdidier, A., Zoete, V., and Michielin, O. (2011). SwissDock, a protein-small molecule docking web service based on EADock DSS. Nucleic Acids Res 39, W270-277.
Group, E. (2021). Evaluating Progestogens for Preventing Preterm birth International Collaborative (EPPPIC): meta-analysis of individual participant data from randomised controlled trials. Lancet 397, 1183-1194.
Guney, E., Menche, J., Vidal, M., and Barabasi, A. L. (2016). Network-based in silico drug efficacy screening. Nat Commun 7, 10331. Gyamfi-Bannerman, C., and Ananth, C. V. (2014). Trends in spontaneous and indicated preterm delivery among singleton gestations in the United States, 2005-2012. Obstet Gynecol 124, 1069-1074.
Hardy, D. B., Janowski, B. A., Corey, D. R., and Mendelson, C. R. (2006). Progesterone receptor plays a major antiinflammatory role in human myometrial cells by antagonism of nuclear factorkappaB activation of cyclooxygenase 2 expression. Mol Endocrinol 20, 2724-2733.
He, K. M., Zhang, X. Y., Ren, S. Q., and Sun, J. (2016). Deep Residual Learning for Image Recognition. Proc Cvpr leee, 770-778.
Heinz, S., Benner, C., Spann, N., Bertolino, E., Lin, Y. C., Laslo, P., Cheng, J. X., Murre, C., Singh, H., and Glass, C. K. (2010). Simple combinations of lineage-determining transcription factors prime cis-regulatory elements required for macrophage and B cell identities. Mol Cell 38, 576-589.
Hoffman, M. K., Goudar, S. S., Kodkany, B. S., Metgud, M., Somannavar, M., Okitawutshu, J., Lokangaka, A., Tshefu, A., Bose, C. L., Mwapule, A., et al. (2020). Low-dose aspirin for the prevention of preterm delivery in nulliparous women with a singleton pregnancy (ASPIRIN): a randomised, double-blind, placebo-controlled trial. Lancet 395, 285-293.
Hong, X. M., Hao, K., Ji, H. K., Peng, S. N., Sherwood, B., Di Narzo, A., Tsai, H. J., Liu, X., Burd, I., Wang, G. Y., et al. (2017). Genome-wide approach identifies a novel gene-maternal prepregnancy BMI interaction on preterm birth. Nature Communications 8.
Huang, N., Lee, I., Marcotte, E. M., and Hurles, M. E. (2010). Characterising and predicting haploinsufficiency in the human genome. PLOS Genet 6, e1001154.
Karczewski, K. J., Francioli, L. C., Tiao, G., Cummings, B. B., Alfoldi, J., Wang, Q., Collins, R. L., Laricchia, K. M., Ganna, A., Birnbaum, D. P., et al. (2020). The mutational constraint spectrum quantified from variation in 141,456 humans. Nature 581, 434-443.
Khanjani, S., Kandola, M. K., Lindstrom, T. M., Sooranna, S. R., Melchionda, M., Lee, Y. S., Terzidou, V., Johnson, M. R., and Bennett, P. R. (2011). NF-kappaB regulates a cassette of immune/inflammatory genes in human pregnant myometrium at term. J Cell Mol Med 15, 809824.
Kita, T., Hata, Y., Arita, R., Kawahara, S., Miura, M., Nakao, S., Mochizuki, Y., Enaida, H., Goto, Y., Shimokawa, H., et al. (2008). Role of TGF-beta in proliferative vitreoretinal diseases and ROCK as a therapeutic target. Proc Natl Acad Sci USA 105, 17504-17509.
Kuleshov, M. V., Jones, M. R., Rouillard, A. D., Fernandez, N. F., Duan, Q., Wang, Z., Koplev, S., Jenkins, S. L., Jagodnik, K. M., Lachmann, A., et al. (2016). Enrichr: a comprehensive gene set enrichment analysis web server 2016 update. Nucleic Acids Res 44, W90-97.
Kyathanahalli, C., Organ, K., Moreci, R. S., Anamthathmakula, P., Hassan, S. S., Caritis, S. N., Jeyasuria, P., and Condon, J. C. (2015). Uterine endoplasmic reticulum stress-unfolded protein response regulation of gestational length is caspase-3 and -7-dependent. Proc Natl Acad Sci USA 112, 14090-14095.
Lee, A. C., Blencowe, H., and Lawn, J. E. (2019). Small babies, big numbers: global estimates of preterm birth. Lancet Glob Health 7, e2-e3.
Lek, M., Karczewski, K. J., Minikel, E. V., Samocha, K. E., Banks, E., Fennell, T., O'Donnell-Luria, A. H., Ware, J. S., Hill, A. J., Cummings, B. B., et al. (2016). Analysis of protein-coding genetic variation in 60,706 humans. Nature 536, 285-291.
Li, J., Hong, X., Mesiano, S., Muglia, L. J., Wang, X., Snyder, M., Stevenson, D. K., and Shaw, G. M. (2018). Natural Selection Has Differentiated the Progesterone Receptor among Human Populations. Am J Hum Genet 103, 45-57.
Li, L., Chen, Q., Yu, Y., Chen, H., Lu, M., Huang, Y., Li, P., and Chang, H. (2020). RKI-1447 suppresses colorectal carcinoma cell growth via disrupting cellular bioenergetics and mitochondrial dynamics. J Cell Physiol 235, 254-266.
Li, Y., Je, H. D., Malek, S., and Morgan, K. G. (2004). Role of ERK1/2 in uterine contractility and preterm labor in rats. Am J Physiol Regul Integr Comp Physiol 287, R328-335.
Lindstrom, T. M., and Bennett, P. R. (2005). The role of nuclear factor kappa B in human labour. Reproduction 130, 569-581.
Loftin, R. W., Habli, M., Snyder, C. C., Cormier, C. M., Lewis, D. F., and Defranco, E. A. (2010). Late preterm birth. Rev Obstet Gynecol 3, 10-19.
Love, M. I., Huber, W., and Anders, S. (2014). Moderated estimation of fold change and dispersion for RNA-seq data with DESeq2. Genome Biol 15, 550.
Machiela, M. J., and Chanock, S. J. (2015). LDlink: a web-based application for exploring population-specific haplotype structure and linking correlated alleles of possible functional variants. Bioinformatics 31, 3555-3557.
Manuck, T. A., Major, H. D., Varner, M. W., Chettier, R., Nelson, L., and Esplin, M. S. (2010). Progesterone receptor genotype, family history, and spontaneous preterm birth. Obstet Gynecol 115, 765-770.
McKenna, A., Hanna, M., Banks, E., Sivachenko, A., Cibulskis, K., Kernytsky, A., Garimella, K., Altshuler, D., Gabriel, S., Daly, M., et al. (2010). The Genome Analysis Toolkit: a MapReduce framework for analyzing next-generation DNA sequencing data. Genome Res 20, 1297-1303.
Meis, P. J., Klebanoff, M., Thom, E., Dombrowski, M. P., Sibai, B., Moawad, A. H., Spong, C. Y., Hauth, J. C., Miodovnik, M., Varner, M. W., et al. (2003). Prevention of recurrent preterm delivery by 17 alpha-hydroxyprogesterone caproate. N Engl J Med 348, 2379-2385.
Mendelson, C. R., Gao, L., and Montalbano, A. P. (2019). Multifactorial Regulation of Myometrial Contractility During Pregnancy and Parturition. Front Endocrinol (Lausanne) 10, 714.
Merlino, A. A., Welsh, T. N., Tan, H., Yi, L. J., Cannon, V., Mercer, B. M., and Mesiano, S. (2007). Nuclear progesterone receptors in the human pregnancy myometrium: evidence that parturition involves functional progesterone withdrawal mediated by increased expression of progesterone receptor-A. J Clin Endocrinol Metab 92, 1927-1933.
Morselli Gysi, D., do Valle, I., Zitnik, M., Ameli, A., Gan, X., Varol, O., Ghiassian, S. D., Patten, J. J., Davey, R. A., Loscalzo, J., et al. (2021). Network medicine framework for identifying drugrepurposing opportunities for COVID-19. Proc Natl Acad Sci USA 118.
Nadeem, L., Shynlova, O., Matysiak-Zablocki, E., Mesiano, S., Dong, X., and Lye, S. (2016). Molecular evidence of functional progesterone withdrawal in human myometrium. Nat Commun 7, 11565.
Nadeem, L., Shynlova, O., Mesiano, S., and Lye, S. (2017). Progesterone Via its Type-A Receptor Promotes Myometrial Gap Junction Coupling. Sci Rep 7, 13357.
Olson, D. M. (2003). The role of prostaglandins in the initiation of parturition. Best Pract Res Clin Obstet Gynaecol 17, 717-730.
Otasek, D., Morris, J. H., Boucas, J., Pico, A. R., and Demchak, B. (2019). Cytoscape Automation: empowering workflow-based network analysis. Genome Biol 20, 185.
Patel, R. A., Forinash, K. D., Pireddu, R., Sun, Y., Sun, N., Martin, M. P., Schonbrunn, E., Lawrence, N. J., and Sebti, S. M. (2012). RKI-1447 is a potent inhibitor of the Rho-associated ROCK kinases with anti-invasive and antitumor activities in breast cancer. Cancer Res 72, 5025-5034.
Peavey, M. C., Wu, S. P., Li, R., Liu, J., Emery, O. M., Wang, T., Zhou, L., Wetendorf, M., Yallampalli, C., Gibbons, W. E., et al. (2021). Progesterone receptor isoform B regulates the Oxtr-Plcl2-Trpc3 pathway to suppress uterine contractility. Proc Natl Acad Sci USA 118.
Purcell, S., Neale, B., Todd-Brown, K., Thomas, L., Ferreira, M. A., Bender, D., Maller, J., Sklar, P., de Bakker, P. I., Daly, M. J., et al. (2007). PLINK: a tool set for whole-genome association and population-based linkage analyses. Am J Hum Genet 81, 559-575.
Quinlan, A. R., and Hall, I. M. (2010). BEDTools: a flexible suite of utilities for comparing genomic features. Bioinformatics 26, 841-842.
Raudvere, U., Kolberg, L., Kuzmin, I., Arak, T., Adler, P., Peterson, H., and Vilo, J. (2019). g:Profiler: a web server for functional enrichment analysis and conversions of gene lists (2019 update). Nucleic Acids Res 47, W191-W198.
Rehman, K. S., Yin, S., Mayhew, B. A., Word, R. A., and Rainey, W. E. (2003). Human myometrial adaptation to pregnancy: cDNA microarray gene expression profiling of myometrium from nonpregnant and pregnant women. Mol Hum Reprod 9, 681-700.
Romero, R., Dey, S. K., and Fisher, S. J. (2014). Preterm labor: one syndrome, many causes. Science 345, 760-765.
Ruiz, C., Zitnik, M., and Leskovec, J. (2021). Identification of disease treatment mechanisms through the multiscale interactome. Nat Commun 12, 1796.
Sakabe, N. J., Aneas, I., Knoblauch, N., Sobreira, D. R., Clark, N., Paz, C., Horth, C., Ziffra, R., Kaur, H., Liu, X., et al. (2020). Transcriptome and regulatory maps of decidua-derived stromal cells inform gene discovery in preterm birth. Sci Adv 6.
Schaub, M. A., Boyle, A. P., Kundaje, A., Batzoglou, S., and Snyder, M. (2012). Linking disease associations with regulatory information in the human genome. Genome Res 22, 1748-1759. Soloff, M. S., Cook, D. L., Jr., Jeng, Y. J., and Anderson, G. D. (2004). In situ analysis of interleukin-1-induced transcription of cox-2 and il-8 in cultured human myometrial cells. Endocrinology 145, 1248-1254.
Stanfield, Z., Amini, P., Wang, J., Yi, L., Tan, H., Chance, M. R., Koyuturk, M., and Mesiano, S. (2019a). Interplay of transcriptional signaling by progesterone, cyclic AMP, and inflammation in myometrial cells: implications for the control of human parturition. Mol Hum Reprod 25, 408-422.
Stanfield, Z., Lai, P. F., Lei, K., Johnson, M. R., Blanks, A. M., Romero, R., Chance, M. R., Mesiano, S., and Koyuturk, M. (2019b). Myometrial Transcriptional Signatures of Human Parturition. Front Genet 10, 185.
Stewart, L. A., Simmonds, M., Duley, L., Llewellyn, A., Sharif, S., Walker, R. A. E., Beresford, L., Wright, K., Aboulghar, M. M., Alfirevic, Z., et al. (2021). Evaluating Progestogens for Preventing Preterm birth International Collaborative (EPPPIC): meta-analysis of individual participant data from randomised controlled trials. Lancet 397, 1183-1194.
Suresh, A., Subedi, K., Kyathanahalli, C., Jeyasuria, P., and Condon, J. C. (2013). Uterine endoplasmic reticulum stress and its unfolded protein response may regulate caspase 3 activation in the pregnant mouse uterus. PLOS One 8, e75152.
Sweeney, H. L. (1998). Regulation and tuning of smooth muscle myosin. Am J Respir Crit Care Med 158, S95-99.
Tan, H., Yi, L., Rote, N. S., Hurd, W. W., and Mesiano, S. (2012). Progesterone receptor-A and B have opposite effects on proinflammatory gene expression in human myometrial cells: implications for progesterone actions in human pregnancy and parturition. J Clin Endocrinol Metab 97, E719-730.
Tarasov, A., Vilella, A. J., Cuppen, E., Nijman, I. J., and Prins, P. (2015). Sambamba: fast processing of NGS alignment formats. Bioinformatics 31, 2032-2034.
Van der Auwera, G. A., Carneiro, M. O., Hartl, C., Poplin, R., Del Angel, G., Levy-Moonshine, A., Jordan, T., Shakir, K., Roazen, D., Thibault, J., et al. (2013). From FastQ data to high confidence variant calls: the Genome Analysis Toolkit best practices pipeline. Curr Protoc Bioinformatics 43, 11 10 11-11 10 33.
Wang, C., and Blei, D. M. (2013). Variational Inference in Nonconjugate Models. J Mach Learn Res 14, 1005-1031.
Wang, C., and Li, J. (2020). A deep learning framework identifies pathogenic noncoding somatic mutations from personal prostate cancer genomes. Cancer Res.
Wang, G., Oh, D. H., and Dassanayake, M. (2020a). GOMCL: a toolkit to cluster, evaluate, and extract non-redundant associations of Gene Ontology-based functions. BMC Bioinformatics 21, 139.
Wang, J., and Jiang, W. (2020). The Effects of RKI-1447 in a Mouse Model of Nonalcoholic Fatty Liver Disease Induced by a High-Fat Diet and in HepG2 Human Hepatocellular Carcinoma Cells Treated with Oleic Acid. Med Sci Monit 26, e919220.
Wang, T., Hoekzema, K., Vecchio, D., Wu, H., Sulovari, A., Coe, B. P., Gillentine, M. A., Wilfert, A. B., Perez-Jurado, L. A., Kvarnung, M., et al. (2020b). Large-scale targeted sequencing identifies risk genes for neurodevelopmental disorders. Nat Commun 11, 4932.
Wu, S. P., Anderson, M. L., Wang, T., Zhou, L., Emery, O. M., Li, X., and DeMayo, F. J. (2020). Dynamic transcriptome, accessible genome, and PGR cistrome profiles in the human myometrium. FASEB J 34, 2252-2268.
Ying, L., Becard, M., Lyell, D., Han, X., Shortliffe, L., Husted, C. I., Alvira, C. M., and Cornfield, D. N. (2015). The transient receptor potential vanilloid 4 channel modulates uterine tone during pregnancy. Sci Transl Med 7, 319ra204.
Yuan, W., and Lopez Bernal, A. (2007). Cyclic AMP signalling pathways in the regulation of uterine relaxation. BMC Pregnancy Childbirth 7 Suppl 1, S10.
Zhang, G., Feenstra, B., Bacelis, J., Liu, X., Muglia, L. M., Juodakis, J., Miller, D. E., Litterman, N., Jiang, P. P., Russell, L., et al. (2017). Genetic Associations with Gestational Duration and Spontaneous Preterm Birth. N Engl J Med 377, 1156-1167.
Zhang, Y., Liu, T., Meyer, C. A., Eeckhoute, J., Johnson, D. S., Bernstein, B. E., Nusbaum, C., Myers, R. M., Brown, M., Li, W., et al. (2008). Model-based analysis of ChIP-Seq (MACS). Genome Biol 9, R137.
Zhou, J., Theesfeld, C. L., Yao, K., Chen, K. M., Wong, A. K., and Troyanskaya, O. G. (2018). Deep learning sequence-based ab initio prediction of variant effects on expression and disease risk. Nat Genet 50, 1171-1179.
Ziegler, R., Hausermann, F., Kirchner, S., and Polonchuk, L. (2021). Cardiac Safety of Kinase Inhibitors—Improving Understanding and Prediction of Liabilities in Drug Discovery Using Human Stem Cell-Derived Models. Front Cardiovasc Med 8, 639824.

TABLE 1
Summary of the identified genomic loci in spontaneous preterm labor.

| | A | A | | | DEEP+ | Distance |
|---|---|---|---|---|---|---|
| POS | 1 | 2 | rsID | Gene | Score | to TSS |
| chr1:201474485 | G | A | rs78433503 | CSRP1 | 5.73619727 | 1482 |
| chr9:35700150 | T | C | rs2295794 | TPM2 | 5.323764086 | −10097 |
| chrX:13005432 | A | G | rs9699155 | TMSB4X | 7.956093792 | 12203 |
| chr15:63313011 | T | C | rs8040022 | TPM1 | 7.707867632 | −21935 |
| chrX:12997469 | C | T | rs1483191 | TMSB4X | 6.318082521 | 4240 |
| chr7:94025737 | G | A | rs79174778 | COL1A2 | 9.920722442 | 1864 |
| chrX:12978836 | C | T | rs66475331 | TMSB4X | 5.619960353 | −14393 |
| chr2:189837298 | A | G | rs11887092 | COL3A1 | 6.639306694 | −1801 |
| chr7:5581594 | G | A | rs6963020 | ACTB | 8.022847734 | −11362 |
| chr16:15948768 | G | T | rs9925813 | MYH11 | 6.202066913 | 2117 |
| chr12:91965005 | A | G | rs187057451 | DCN | 6.30714817 | −388411 |
| chr3:123557081 | G | A | rs2682203 | MYLK | 7.573892214 | 46098 |
| chr2:217579874 | C | G | rs17824343 | IGFBP5 | 7.469307519 | −19602 |
| chr12:91729020 | T | C | rs75582404 | DCN | 7.337730881 | −152426 |
| chr2:74127845 | G | T | rs1618091 | ACTG2 | 4.172708038 | 7710 |
| chr4:88444670 | G | A | rs17012787 | SPARCL1 | 5.856601801 | 5985 |
| chr16:15875011 | C | T | rs13339162 | MYH11 | 5.284259599 | 75874 |
| chr9:35683148 | T | C | rs56249943 | TPM2 | 4.354197681 | 6774 |
| chr2:85140148 | C | A | rs59340165 | TMSB10 | 7.128499011 | 7368 |
| chr10:45134272 | G | A | rs11813145 | CXCL12 | 8.484083692 | −253727 |
| chr3:123566341 | A | G | rs2700396 | MYLK | 5.977850677 | 36838 |
| chr5:149765744 | C | T | rs11742201 | CD74 | 6.565805417 | 26575 |
| chr7:134413003 | C | T | rs4732051 | CALD1 | 6.738137205 | −51382 |
| chr3:123610885 | A | G | rs111330081 | MYLK | 6.137120444 | −7706 |
| chr4:169622211 | C | A | rs76999605 | PALLD | 7.085550227 | 69482 |
| chr11:332439 | A | G | rs77358882 | IFITM3 | 4.753409177 | −11579 |
| chr10:90726381 | A | T | rs76671311 | ACTA2 | 4.226321541 | −13851 |
| chr4:58005459 | G | A | rs4444899 | IGFBP7 | 6.253448577 | −28908 |
| chr2:189826414 | C | G | rs115301620 | COL3A1 | 4.867622289 | −12685 |
| chr12:91614652 | C | A | rs1797250 | DCN | 9.290947655 | −38058 |
| chr12:91803151 | G | T | rs116712810 | DCN | 4.675152558 | −226557 |
| chr15:64548785 | G | A | rs139933295 | PPIB | 6.678992361 | −93431 |
| chr7:134420613 | A | C | rs7779553 | CALD1 | 7.848002433 | −43772 |
| chr20:35159605 | T | C | rs140757851 | MYL9 | 4.469637675 | −10317 |
| chr12:15038788 | T | C | rs1800801 | MGP | 7.949628457 | 0 |
| chr12:91549515 | C | A | rs3138251 | DCN | 6.929649031 | 22908 |
| chr15:63232404 | A | C | rs149060116 | TPM1 | 5.207674839 | −102542 |
| chr16:15883481 | T | C | rs72772068 | MYH11 | 4.363506939 | 67404 |
| chr12:56562404 | T | C | rs143891417 | MYL6 | 4.58147265 | 10261 |
| chr5:172192868 | T | C | rs322353 | DUSP1 | 5.155063961 | 5330 |
| chr6:29914777 | G | C | rs17179578 | HLA-A | 7.116443673 | 4468 |
| chr4:88462729 | G | A | rs7681694 | SPARCL1 | 6.016013548 | −12074 |
| chr3:123638476 | T | C | rs2682253 | MYLK | 4.386463128 | −35297 |
| chr2:238760118 | A | G | rs1198823 | RAMP1 | 6.500957392 | −8148 |
| chr12:91575985 | C | A | rs13312824 | DCN | 4.543476552 | 609 |
| chr10:73637359 | C | T | rs116994293 | PSAP | 5.338303242 | −26351 |
| chr12:91626274 | G | A | rs7303223 | DCN | 5.889091422 | −49680 |
| chr7:94029982 | T | C | rs3814967 | COL1A2 | 4.416283462 | 6109 |
| chr3:123635469 | T | C | rs9822006 | MYLK | 4.446255424 | −32290 |
| chr16:15965344 | C | T | rs141484631 | MYH11 | 6.260615909 | −14457 |
| chr19:3987202 | A | G | rs74172614 | EEF2 | 5.766653195 | −1741 |
| chr22:36749600 | T | C | rs117573719 | MYH9 | 6.234811246 | 34512 |
| chr3:123549230 | A | C | rs2700349 | MYLK | 4.427524447 | 53949 |
| chr4:88404513 | T | A | rs150025764 | SPARCL1 | 4.971677975 | 46142 |
| chr10:73614416 | G | A | rs148235116 | PSAP | 5.264201052 | −3408 |
| chr12:91614394 | G | T | rs11106050 | DCN | 5.971830405 | −37800 |
| chr15:63236912 | T | C | rs146060900 | TPM1 | 4.258270484 | −98034 |
| chr15:63327384 | A | G | rs77445262 | TPM1 | 5.981298611 | −7562 |
| chr1:201452356 | G | A | rs586507 | CSRP1 | 5.325340331 | 13345 |
| chr3:112353995 | C | T | rs9813486 | CCDC80 | 5.359258559 | 5995 |
| chr22:31447612 | G | T | rs185238346 | SMTN | 5.543151647 | −29692 |
| chr6:31327190 | C | A | rs3997998 | HLA-B | 5.848881155 | −2234 |
| chr10:45123671 | C | T | rs12247874 | CXCL12 | 5.67342543 | −243126 |
| chr4:122600023 | T | C | rs10008313 | ANXA5 | 5.679733406 | 18112 |
| chr17:16287454 | C | T | rs10432024 | UBB | 5.802535292 | 2675 |
| chr22:36828935 | G | A | rs73407609 | MYH9 | 7.127518728 | −44823 |
| chr3:112353641 | A | G | rs185299757 | CCDC80 | 9.391709887 | 6349 |
| chr7:5577671 | C | T | rs138786271 | ACTB | 4.651038609 | −7439 |
| chr4:169433444 | C | T | rs72695199 | PALLD | 5.826104041 | 15241 |
| chr4:58069873 | G | T | rs184377149 | IGFBP7 | 5.168228563 | −93322 |
| chr12:125383040 | T | C | rs7308593 | UBC | 7.207077472 | 16156 |
| chr13:48800340 | G | T | rs80316732 | ITM2B | 6.24867334 | −7002 |
| chr11:57372974 | A | G | rs12806113 | SERPING1 | 6.117499787 | 7290 |
| chr12:92028804 | C | T | rs146385166 | DCN | 4.675368024 | −452210 |
| chr12:15023557 | A | G | rs2430731 | MGP | 6.69521057 | 15231 |
| chr3:123354268 | G | C | rs9850230 | MYLK | 4.050620645 | −14873 |
| chr1:145436886 | G | T | rs2236566 | TXNIP | 5.995515324 | −1604 |
| chr7:134443975 | T | G | rs1026276 | CALD1 | 5.638677329 | −20410 |
| chr10:45125483 | T | C | rs17409431 | CXCL12 | 5.374330608 | −244938 |
| chr4:88438944 | G | C | rs143447267 | SPARCL1 | 5.199601771 | 11711 |
| chr7:94034689 | T | C | rs28417792 | COL1A2 | 4.257111102 | 10816 |
| chr9:123944635 | G | A | rs2057471 | GSN | 7.302339547 | −19126 |
| chr20:35183699 | T | C | rs6071089 | MYL9 | 4.497636966 | 13777 |
| chr11:2371003 | T | C | rs60469226 | CD81 | 6.494341847 | −26404 |
| chr12:91612844 | C | A | rs184338867 | DCN | 5.430267755 | −36250 |
| chr10:79315061 | T | A | rs183239077 | KCNMA1 | 7.388507165 | 82505 |
| chr4:169501161 | G | A | rs62335500 | PALLD | 6.830204986 | −51568 |
| chr12:15029598 | G | A | rs2900342 | MGP | 6.052931286 | 9190 |
| chr6:31335096 | G | A | rs7767216 | HLA-B | 10.81717772 | −10140 |
| chr10:45063108 | G | T | rs10793546 | CXCL12 | 6.731878838 | −182563 |
| chr6:169044284 | T | A | rs12196836 | SMOC2 | 7.059932277 | 202420 |
| chrX:135151989 | A | C | rs138540024 | FHL1 | 4.826591101 | −76872 |
| chr4:169792806 | A | G | rs2062590 | PALLD | 5.111512362 | 39560 |
| chr5:85849546 | T | C | rs148871316 | COX7C | 5.727465649 | −64212 |
| chr4:58004606 | C | T | rs13141383 | IGFBP7 | 5.035069082 | −28055 |
| chr7:134441577 | T | C | rs10238683 | CALD1 | 6.697757135 | −22808 |
| chr15:63286519 | C | G | rs143855885 | TPM1 | 3.845860772 | −48427 |
| chr7:94035892 | C | T | rs3763466 | COL1A2 | 4.861348921 | 12019 |
| chrX:135254671 | T | C | rs3753172 | FHL1 | 5.340611367 | 2875 |
| chr15:64514904 | G | A | rs140434152 | PPIB | 7.469411608 | −59550 |
| chr10:29920219 | C | T | rs141613435 | SVIL | 8.172799684 | 3681 |
| chr4:169624278 | C | T | rs181379090 | PALLD | 4.928106037 | 71549 |
| chr5:172199366 | A | G | rs13184134 | DUSP1 | 4.806757718 | −1168 |
| chr22:36773445 | C | G | rs142264658 | MYH9 | 5.070775608 | 10667 |
| chr2:189821869 | A | G | rs2222108 | COL3A1 | 4.194555264 | −17230 |
| chr21:41180064 | C | T | rs79805783 | PCP4 | 5.266298372 | −59300 |
| chr7:75928409 | A | T | rs149989159 | HSPB1 | 5.599239506 | −3581 |
| chr2:85139584 | C | T | rs1814590 | TMSB10 | 5.839492902 | 6804 |
| chr21:41344565 | C | T | rs1235554 | PCP4 | 5.161234625 | 105201 |
| chr6:31342420 | G | A | rs2395491 | HLA-B | 5.311417636 | −17464 |
| chr8:109317304 | T | C | rs140998692 | EIF3E | 8.643119168 | −56358 |
| chr4:10186208 | A | G | rs139593010 | WDR1 | 8.355983384 | −67785 |
| chr11:2378970 | A | G | rs148321757 | CD81 | 4.447165849 | −18437 |
| chr6:29910602 | G | A | rs41552219 | HLA-A | 5.124663264 | 293 |
| chr7:94070053 | A | C | rs118094537 | COL1A2 | 4.792224411 | 46180 |
| chr4:6628677 | T | C | rs4689023 | MRFAP1 | 6.768582165 | −13141 |
| chr4:88453631 | A | C | rs12501773 | SPARCL1 | 4.530116245 | −2976 |
| chr12:125382112 | T | C | rs10846766 | UBC | 5.47842551 | 17084 |
| chr12:56563278 | T | C | rs113256868 | MYL6 | 5.260965722 | 11135 |
| chr16:15942434 | A | G | rs28535135 | MYH11 | 4.271416031 | 8451 |
| chr5:149767097 | G | T | rs1864957 | CD74 | 4.696830064 | 25222 |
| chr15:63321448 | T | A | rs72743205 | TPM1 | 3.823913308 | −13498 |
| chr2:85124572 | A | C | rs12477824 | TMSB10 | 6.715759281 | −8208 |
| chr1:59239988 | G | A | rs33989940 | JUN | 6.248078586 | 9731 |
| chr11:111774743 | A | G | rs3944619 | CRYAB | 5.881642001 | 6720 |
| chr11:111773296 | C | T | rs11214037 | CRYAB | 5.885530868 | 8167 |
| chr5:85824690 | C | T | rs181570175 | COX7C | 6.263440693 | −89068 |
| chr12:91642694 | C | T | rs190148519 | DCN | 5.364571193 | −66100 |
| chr5:151073377 | G | T | rs6579889 | SPARC | 6.030892581 | −6901 |
| chr14:69416346 | G | A | rs8010531 | ACTN1 | 5.969597921 | 29673 |
| chr17:1681048 | G | C | rs78848122 | SERPINF1 | 5.193670867 | 6083 |
| chr10:73642081 | G | A | rs147412411 | PSAP | 4.756641318 | −31073 |
| chr21:47516351 | T | C | rs914244 | COL6A2 | 4.291272447 | −1675 |
| chr6:31327177 | C | T | rs2394986 | HLA-B | 6.08807046 | −2221 |
| chrX:13009145 | G | A | rs59671287 | TMSB4X | 5.967532946 | 15916 |
| chr2:232547863 | A | G | rs11692904 | PTMA | 6.257507652 | −25372 |
| chr10:45003677 | T | C | rs139572265 | CXCL12 | 4.771225825 | −123132 |
| chr2:47467210 | A | G | rs55736752 | CALM2 | 5.036032009 | −63135 |
| chr4:88447303 | G | A | rs6820415 | SPARCL1 | 5.738483167 | 3352 |
| chr4:57999586 | T | C | rs12512048 | IGFBP7 | 4.770264104 | −23035 |
| chr12:15032020 | T | C | rs11056227 | MGP | 5.088517726 | 6768 |
| chr4:10277869 | C | T | rs11736814 | WDR1 | 5.461314328 | −159446 |
| chr4:169624249 | C | G | rs148641359 | PALLD | 4.687049296 | 71520 |
| chr11:324635 | G | A | rs6598042 | IFITM3 | 4.168994427 | −3775 |
| chr1:153473805 | G | T | rs193139279 | S100A6 | 5.723772682 | 34662 |
| chr11:117062295 | A | G | rs148801054 | TAGLN | 4.959515668 | −7715 |
| chr19:11657814 | T | C | rs113148999 | CNN1 | 3.670141548 | 8148 |
| chr12:21814956 | T | A | rs111666849 | LDHB | 5.510450695 | −4167 |
| chr20:35178164 | G | A | rs77354216 | MYL9 | 3.688796349 | 8242 |
| chr21:47450492 | C | A | rs149799212 | COL6A1 | 4.124177666 | 48829 |
| chr4:10168999 | G | C | rs1001217 | WDR1 | 6.126385797 | −50576 |
| chr4:88459017 | A | C | rs10000576 | SPARCL1 | 4.202675903 | −8362 |
| chr5:130467549 | T | C | rs74590752 | HINT1 | 7.323950008 | 33400 |
| chr15:63254451 | T | C | rs187246359 | TPM1 | 4.875620306 | −80495 |
| chr2:238802299 | G | A | rs6760549 | RAMP1 | 5.205103466 | 34033 |
| chr15:45001889 | C | T | rs182087441 | B2M | 4.934326607 | −1826 |
| chr7:134406961 | T | G | rs12540516 | CALD1 | 4.451399259 | −57424 |
| chr1:201474690 | G | C | rs3767526 | CSRP1 | 3.651091233 | 1277 |
| chr2:218810474 | C | T | rs11892874 | TNS1 | 7.263629958 | −1681 |
| chr9:76293168 | C | T | rs76672098 | ANXA1 | 8.198616989 | 526387 |
| chr11:119361560 | C | T | rs80095119 | THY1 | 6.890668236 | −65865 |
| chr2:74130557 | C | G | rs1726024 | ACTG2 | 3.662671968 | 10422 |
| chr7:134429523 | C | A | rs55663556 | CALD1 | 5.612354688 | −34862 |
| chr5:85836953 | A | C | rs116275475 | COX7C | 5.229894142 | −76805 |
| chr14:69321164 | C | A | rs59506075 | ZFP36L1 | 4.806176964 | −58204 |
| chr12:15022686 | A | C | rs2098436 | MGP | 4.921023622 | 16102 |
| chr16:15870530 | C | T | rs189703801 | MYH11 | 4.001781549 | 80355 |
| chr2:217614730 | A | T | rs6435952 | IGFBP5 | 4.028551629 | −54458 |
| chr4:80878257 | T | C | rs72871890 | ANTXR2 | 7.521204246 | 116126 |
| chr21:27341210 | A | T | rs9636775 | APP | 6.806303412 | 171498 |
| chr11:122991864 | A | G | rs191440463 | HSPA8 | 5.360153317 | −59020 |
| chr16:15934868 | C | T | rs61611600 | MYH11 | 3.95747542 | 16017 |
| chr21:47495805 | A | C | rs13433442 | COL6A2 | 4.428582309 | −22221 |
| chr4:10204496 | T | C | rs10030782 | WDR1 | 7.488360032 | −86073 |
| chr4:88447335 | T | C | rs6845839 | SPARCL1 | 4.492454343 | 3320 |
| chr7:134417820 | A | G | rs10258722 | CALD1 | 4.63221848 | −46565 |
| chr14:69301190 | T | C | rs11847206 | ZFP36L1 | 4.838693502 | −38230 |
| chr6:29922248 | T | C | rs185637437 | HLA-A | 5.359897115 | 11939 |
| chr10:79505640 | A | G | rs79298338 | KCNMA1 | 5.228733801 | −108074 |
| chr16:15850912 | C | T | rs12919484 | MYH11 | 4.527611285 | 99973 |
| chr10:45137400 | T | C | rs11598178 | CXCL12 | 4.5846168 | −256855 |
| chrX:80513891 | C | T | rs138302261 | SH3BGRL | 6.136492509 | 56290 |
| chr2:37911588 | C | A | rs11896756 | CDC42EP3 | 6.19073497 | −11910 |
| chr15:63279249 | C | T | rs144342807 | TPM1 | 3.74014318 | −55697 |
| chr9:124012578 | G | A | rs146479522 | GSN | 4.3499282 | −17862 |
| chr22:36796613 | C | T | rs75864797 | MYH9 | 5.748349018 | −12501 |
| chr17:76943631 | C | T | rs147808424 | TIMP2 | 4.387287125 | −22162 |
| chr2:217561467 | G | A | rs1978346 | IGFBP5 | 3.948567361 | −1195 |
| chr5:169807578 | G | T | rs184241710 | KCNMB1 | 5.800468549 | 8793 |
| chr2:20664705 | C | T | rs342114 | RHOB | 6.117430769 | 17870 |
| chr21:47447480 | T | C | rs66956709 | COL6A1 | 6.12058118 | 45817 |
| chr11:68780892 | C | T | rs183162581 | MRGPRF | 7.161822207 | −42 |
| chr15:63299818 | T | A | rs28572649 | TPM1 | 5.53289555 | −35128 |
| chr4:6629306 | G | A | rs13147808 | MRFAP1 | 5.228693411 | −12512 |
| chr7:134430514 | T | C | rs1993201 | CALD1 | 4.363200637 | −33871 |
| chr5:85800397 | A | T | rs115990936 | COX7C | 5.132476885 | −113361 |
| chr4:169500166 | A | G | rs17054359 | PALLD | 5.040901316 | −52563 |
| chr21:27467908 | G | A | rs67792436 | APP | 5.020644361 | 44800 |
| chr4:10198950 | T | C | rs80079368 | WDR1 | 5.462930528 | −80527 |
| chr10:45041058 | G | T | rs79569730 | CXCL12 | 4.791548543 | −160513 |
| chr7:115894558 | G | A | rs62475211 | TES | 5.613938957 | 31551 |
| chr4:10120609 | T | C | rs12374320 | WDR1 | 7.549879886 | −2186 |
| chr13:48743299 | C | T | rs9526459 | ITM2B | 4.949803483 | −64043 |
| chr21:27313241 | C | T | rs182740128 | APP | 5.85590288 | 199467 |
| chr5:149784414 | C | T | rs11749903 | CD74 | 4.592745397 | 7905 |
| chr12:91630155 | A | G | rs1522430 | DCN | 5.254157016 | −53561 |
| chr2:238791826 | G | A | rs114665574 | RAMP1 | 5.243586833 | 23560 |
| chr4:80903865 | C | T | rs13107152 | ANTXR2 | 7.80959422 | 90518 |
| chr5:130199506 | T | C | rs115223959 | HINT1 | 7.216687817 | 301443 |
| chr10:97325341 | G | A | rs12359410 | SORBS1 | 7.185457125 | −4213 |
| chr7:115788774 | C | T | rs79760655 | TES | 5.324707497 | −61819 |
| chr12:91717608 | C | T | rs79591020 | DCN | 6.014156388 | −141014 |
| chr1:59213743 | A | G | rs13376301 | JUN | 5.540290251 | 35976 |
| chr16:15863066 | G | A | rs2384936 | MYH11 | 6.401478797 | 87819 |
| chr18:3275596 | A | G | rs58625939 | MYL12B | 4.889171448 | 12879 |
| chr2:232567606 | T | A | rs111583165 | PTMA | 4.794518696 | −5629 |
| chr8:109290407 | G | A | rs150308859 | EIF3E | 6.174785011 | −29461 |
| chr5:137821276 | G | A | rs114563815 | EGR1 | 5.950547718 | 20108 |
| chr2:219072675 | G | A | rs10209152 | ARPC2 | 5.67760095 | −9237 |
| chr1:22226919 | G | T | rs71637000 | HSPG2 | 5.939366892 | 36884 |
| chr20:35132193 | C | T | rs181774245 | MYL9 | 3.653985858 | −37729 |
| chr1:201458071 | A | T | rs3738283 | CSRP1 | 3.770337738 | 7630 |
| chr15:63302309 | G | A | rs16946255 | TPM1 | 4.906484988 | −32637 |
| chr12:91547030 | G | A | rs3138262 | DCN | 4.687299021 | 25393 |
| chr2:37944425 | T | C | rs17510483 | CDC42EP3 | 6.029753503 | −44747 |
| chr1:40480484 | A | G | rs12758865 | CAP1 | 6.158730537 | −25253 |
| chr2:189822670 | A | G | rs16830914 | COL3A1 | 4.003510594 | −16429 |
| chr11:836227 | A | G | rs28434354 | CD151 | 5.524981692 | 3275 |
| chrX:81343634 | G | A | rs185411635 | SH3BGRL | 7.485460732 | 886033 |
| chr7:94044056 | T | G | rs187676169 | COL1A2 | 3.807439035 | 20183 |
| chr2:189833911 | C | T | rs6434304 | COL3A1 | 4.386527566 | −5188 |
| chr2:217614595 | C | T | rs145058908 | IGFBP5 | 3.926230391 | −54323 |
| chr13:47365114 | G | A | rs116012939 | ESD | 6.697125272 | 6182 |
| chr14:69233163 | A | C | rs142675549 | ZFP36L1 | 4.837217256 | 27468 |
| chr15:63230462 | C | T | rs61601045 | TPM1 | 3.831603304 | −104484 |
| chr5:150552585 | A | G | rs145968489 | ANXA6 | 5.730080251 | −15245 |
| chr6:56731530 | C | T | rs78687000 | DST | 5.002327403 | −14816 |
| chr16:15966612 | T | G | rs112666539 | MYH11 | 5.368349263 | −15725 |
| chr6:31325159 | G | A | rs9266222 | HLA-B | 6.218800098 | −203 |
| chr6:122780651 | T | C | rs74865090 | SERINC1 | 7.272463382 | 12301 |
| chr20:4594466 | T | C | rs6052686 | PRNP | 6.835898217 | −72636 |
| chr20:4488552 | T | C | rs75815088 | PRNP | 7.906247884 | −178550 |
| chr12:91622861 | C | T | rs78540019 | DCN | 8.577134553 | −46267 |
| chr6:44209675 | C | T | rs2039362 | HSP90AB1 | 4.876094051 | −4228 |
| chr21:47504302 | T | C | rs11701379 | COL6A2 | 5.134531744 | −13724 |
| chr20:23633201 | G | C | rs137985733 | CST3 | 5.599852651 | −14609 |
| chr4:10261592 | T | G | rs73222570 | WDR1 | 5.377440602 | −143169 |
| chr2:47449545 | A | G | rs904167 | CALM2 | 5.19034531 | −45470 |
| chr6:29910402 | G | A | rs1136656 | HLA-A | 4.315633597 | 93 |
| chr20:17579529 | C | A | rs8117083 | DSTN | 4.704782665 | 28809 |
| chr11:70199036 | G | C | rs66627443 | CTTN | 6.273634909 | −45576 |
| chrX:135263179 | G | T | rs5930896 | FHL1 | 5.834993534 | 11383 |
| chr2:47440641 | G | C | rs2132924 | CALM2 | 4.669493921 | −36566 |
| chr4:122650773 | A | G | rs13149537 | ANXA5 | 7.241321858 | −32638 |
| chr14:69246986 | G | A | rs76200426 | ZFP36L1 | 6.004084796 | 13645 |
| chr14:69412064 | G | A | rs7145079 | ACTN1 | 4.435010045 | 33955 |
| chr21:27445240 | C | T | rs12483305 | APP | 4.932538408 | 67468 |
| chr14:69264106 | C | A | rs11846601 | ZFP36L1 | 5.391924158 | −1146 |
| chr1:151382872 | C | T | rs74469136 | PSMB4 | 5.757871072 | 10823 |
| chr7:115870265 | C | T | rs10243102 | TES | 5.40382558 | 7258 |
| chr6:29920191 | C | A | rs13201160 | HLA-A | 5.08114143 | 9882 |
| chr4:169411491 | A | G | rs2712132 | PALLD | 4.358307086 | −6712 |
| chr17:1682422 | G | A | rs7214704 | SERPINF1 | 4.59637668 | 7457 |
| chr14:70179104 | T | A | rs144349598 | SRSF5 | 4.984562611 | −54755 |
| chr12:119665964 | A | G | rs2727711 | HSPB8 | 5.346575268 | 49369 |
| chr8:101919321 | C | T | rs931812 | YWHAZ | 5.447588624 | 43478 |
| chr12:98963104 | G | A | rs186184978 | SLC25A3 | 5.453619026 | −24299 |
| chr10:29961099 | G | A | rs1148213 | SVIL | 6.052603163 | −37199 |
| chr5:146894104 | A | C | rs115894330 | DPYSL3 | 5.845622771 | −4473 |
| chr2:238252972 | C | G | rs11897148 | COL6A3 | 6.024796832 | 69835 |
| chr12:118560629 | T | C | rs11068835 | PEBP1 | 5.471949838 | −13300 |
| chr10:88724037 | A | G | rs1873803 | ADIRF | 5.570082553 | −4202 |
| chr12:6871499 | G | A | rs7135106 | PTMS | 4.736301564 | −4030 |
| chr15:39865384 | C | T | rs72725045 | THBS1 | 6.279591322 | −7896 |
| chr5:130165541 | A | T | rs150334478 | HINT1 | 4.989944586 | 335408 |
| chr22:36799354 | C | G | rs146220344 | MYH9 | 5.288970321 | −15242 |
| chr2:85122503 | G | A | rs2886472 | TMSB10 | 5.012517869 | −10277 |
| chr11:35162672 | A | G | rs190663467 | CD44 | 5.99856196 | 1954 |
| chr2:85124131 | T | C | rs13427388 | TMSB10 | 5.094908234 | −8649 |
| chr4:169526644 | G | A | rs62335532 | PALLD | 4.401365034 | −26085 |
| chr4:10272605 | A | C | rs10489074 | WDR1 | 5.627094889 | −154182 |
| chr21:27323504 | G | A | rs170066 | APP | 5.633034073 | 189204 |
| chr1:39512153 | A | G | rs12072299 | NDUFS5 | 6.349178404 | 20131 |
| chr2:189785993 | G | A | rs1554204 | COL3A1 | 4.451725234 | −53106 |
| chr5:137830420 | C | T | rs11741961 | EGR1 | 5.304398388 | 29252 |
| chr6:169043094 | A | G | rs911947 | SMOC2 | 4.815946966 | 201230 |
| chr13:48773294 | T | C | rs78491404 | ITM2B | 4.667161256 | −34048 |
| chr10:75799664 | T | C | rs12782972 | VCL | 6.581934765 | 41790 |
| chr1:22229832 | G | A | rs71569856 | HSPG2 | 6.834396794 | 33971 |
| chr4:108985388 | G | A | rs4613638 | HADH | 7.33300387 | 59568 |
| chr12:21819842 | G | A | rs7316669 | LDHB | 9.05352544 | −9053 |
| chr4:169595152 | A | G | rs2723709 | PALLD | 5.574893057 | 42423 |
| chr8:101972010 | C | G | rs2923784 | YWHAZ | 5.272031911 | −6400 |
| chr10:45049334 | T | C | rs77060696 | CXCL12 | 4.967260286 | −168789 |
| chr6:29908892 | G | A | rs79761237 | HLA-A | 4.257068122 | −1355 |
| chrX:114692478 | A | C | rs2207052 | PLS3 | 7.127316918 | −103023 |
| chr4:58077594 | A | T | rs7439949 | IGFBP7 | 4.089275785 | −101043 |
| chr6:168970508 | C | A | rs6455530 | SMOC2 | 5.024085748 | 128644 |
| chr3:99574232 | G | A | rs793444 | FILIP1L | 7.341390497 | 20785 |
| chr7:134516878 | T | A | rs73724870 | CALD1 | 4.766638577 | 52493 |
| chr12:91613263 | A | C | rs1718520 | DCN | 6.27301337 | −36669 |
| chr2:20238089 | T | C | rs73220358 | LAPTM4A | 7.584337451 | 13300 |
| chr5:130239739 | G | A | rs6883771 | HINT1 | 4.78805864 | 261210 |
| chr2:37933761 | T | A | rs929548 | CDC42EP3 | 6.21051237 | −34083 |
| chr1:202536140 | T | C | rs3767395 | PPP1R12B | 6.695364682 | 26898 |
| chr22:36782627 | G | T | rs140034067 | MYH9 | 6.087932214 | 1485 |
| chr9:75948727 | G | A | rs193022113 | ANXA1 | 5.662424117 | 181946 |
| chr11:74442290 | A | G | rs61897000 | CHRDL2 | 5.377895348 | 181 |
| chrX:135218133 | A | C | rs143640005 | FHL1 | 4.573256512 | −10728 |
| chr12:21858180 | C | T | rs860447 | LDHB | 4.970337929 | −47391 |
| chr11:321235 | G | A | rs56232455 | IFITM3 | 4.051435697 | −375 |
| chr6:56780741 | C | T | rs9475752 | DST | 5.959206112 | 38887 |
| chr6:169040952 | A | G | rs138125909 | SMOC2 | 5.471122642 | 199088 |
| chr12:8804636 | T | C | rs35327409 | MFAP5 | 4.803351909 | 10786 |
| chr8:101745672 | A | G | rs139291301 | PABPC1 | 8.40124147 | −11356 |
| chr10:90725598 | A | G | rs79789848 | ACTA2 | 3.553908655 | −13068 |
| chr1:40487879 | G | A | rs75633494 | CAP1 | 5.653173048 | −17858 |
| chr1:202365332 | G | A | rs12023106 | PPP1R12B | 6.838305853 | 47502 |
| chr6:56537363 | T | C | rs56991168 | DST | 4.893903616 | −29676 |
| chr4:122530913 | T | C | rs189283463 | ANXA5 | 6.036762742 | 87222 |
| chr15:63292674 | C | A | rs145791379 | TPM1 | 4.133324157 | −42272 |
| chr5:149778360 | C | T | rs34133581 | CD74 | 4.309005141 | 13959 |
| chr2:238795298 | G | A | rs74002603 | RAMP1 | 4.702889938 | 27032 |
| chr11:2381595 | C | G | rs74048456 | CD81 | 3.967905119 | −15812 |
| chr10:79071453 | A | G | rs624713 | KCNMA1 | 5.04877897 | 39066 |
| chr6:29905989 | C | T | rs4713264 | HLA-A | 5.373743558 | −4258 |
| chr10:79026150 | C | A | rs74440280 | KCNMA1 | 4.770260118 | 84369 |
| chr1:22241544 | C | T | rs71637001 | HSPG2 | 5.776073411 | 22259 |
| chr11:323139 | C | T | rs73398514 | IFITM3 | 4.466036186 | −2279 |
| chr1:59237432 | G | C | rs2476309 | JUN | 4.513428248 | 12287 |
| chr6:168818952 | T | C | rs9346710 | SMOC2 | 6.824889109 | −22912 |
| chr21:47503286 | A | G | rs59555463 | COL6A2 | 4.901268817 | −14740 |
| chr21:41324325 | G | T | rs2837319 | PCP4 | 4.457275218 | 84961 |
| chr4:58013684 | C | T | rs4864612 | IGFBP7 | 3.979434021 | −37133 |
| chr17:1329282 | G | C | rs186924121 | YWHAE | 5.314508975 | −25726 |
| chr11:8848520 | G | A | rs7127564 | ST5 | 5.309931082 | −15638 |
| chr1:202503077 | T | C | rs7518415 | PPP1R12B | 6.545938348 | −6165 |
| chr11:74429987 | A | G | rs12417156 | CHRDL2 | 5.111934543 | 361 |
| chr20:3672240 | G | A | rs111969446 | ADAM33 | 4.741633758 | −8903 |
| chr6:29907565 | T | C | rs28749124 | HLA-A | 6.876100255 | −2682 |
| chr4:169689516 | T | C | rs17542688 | PALLD | 4.374014398 | −63730 |
| chr4:122556180 | C | T | rs11734352 | ANXA5 | 4.371447866 | 61955 |
| chr2:189836866 | G | A | rs3806465 | COL3A1 | 3.833755743 | −2233 |
| chr2:217623226 | T | G | rs737310 | IGFBP5 | 5.222690357 | −62954 |
| chr1:145444556 | G | T | rs4636400 | TXNIP | 5.474645793 | 5231 |
| chr2:128442063 | G | A | rs55685181 | LIMS2 | 5.521154171 | −2703 |
| chr10:45143639 | G | A | rs922651 | CXCL12 | 5.144271926 | −263094 |
| chr10:90712315 | C | T | rs72809344 | ACTA2 | 3.258346467 | 215 |
| chr20:17567866 | C | T | rs74273007 | DSTN | 4.287575427 | 17146 |
| chr2:37851431 | C | T | rs13405798 | CDC42EP3 | 5.514855739 | 47911 |
| chr8:109308459 | A | G | rs142486869 | EIF3E | 5.103187906 | −47513 |
| chr8:145649365 | G | A | rs149889412 | VPS28 | 7.298087534 | 4566 |
| chr5:149787598 | A | G | rs115781303 | CD74 | 4.543847404 | 4721 |
| chr11:70260598 | T | C | rs34053053 | CTTN | 5.619779979 | 15963 |
| chr5:137803155 | T | G | rs141771089 | EGR1 | 6.11827502 | 1987 |
| chr4:58006556 | C | T | rs6554428 | IGFBP7 | 7.090913989 | −30005 |
| chr4:58079767 | C | T | rs6832042 | IGFBP7 | 3.946667578 | −103216 |
| chr4:88449181 | C | T | rs7671511 | SPARCL1 | 4.893616536 | 1474 |
| chr7:134502306 | C | T | rs17168063 | CALD1 | 4.387721973 | 37921 |
| chr7:134441597 | T | C | rs73446122 | CALD1 | 4.098709763 | −22788 |
| chr12:57124391 | T | C | rs114396866 | NACA | 7.706479989 | −5308 |
| chr5:130426400 | C | G | rs140755884 | HINT1 | 5.401535127 | 74549 |
| chr12:98976004 | T | C | rs12816489 | SLC25A3 | 5.203717744 | −11399 |
| chr22:31486612 | G | C | rs75803893 | SMTN | 4.478610661 | 5630 |
| chr4:88451579 | A | C | rs1031796 | SPARCL1 | 3.819303066 | −924 |
| chr4:186508805 | T | G | rs61744509 | PDLIM3 | 5.166839138 | −52093 |
| chr22:33135768 | T | G | rs732446 | TIMP3 | 6.12009488 | −61034 |
| chr2:232350140 | C | T | rs142504484 | NCL | 4.971580871 | −20945 |
| chr7:115839922 | T | A | rs78244969 | TES | 4.799401183 | −10671 |
| chr9:76089602 | A | G | rs278747 | ANXA1 | 5.916277617 | 322821 |
| chr7:115833686 | C | T | rs78181863 | TES | 5.455617793 | −16907 |
| chr5:151083692 | C | T | rs79878782 | SPARC | 3.815528853 | −17216 |
| chr1:115286071 | G | A | rs72687886 | CSDE1 | 5.204787422 | 14534 |
| chr2:20259224 | T | C | rs193063521 | LAPTM4A | 5.357445481 | −7835 |
| chr4:37898650 | A | C | rs2995926 | TBC1D1 | 7.279461541 | 5945 |
| chr10:97206619 | T | C | rs9633700 | SORBS1 | 4.869794985 | −5682 |
| chr15:64538180 | G | C | rs112049453 | PPIB | 4.442609595 | −82826 |
| chr1:164247402 | G | A | rs1578090 | PBX1 | 5.914551036 | −281195 |
| chr3:123510606 | G | A | rs6785924 | MYLK | 5.509313317 | 92573 |
| chr21:47413105 | T | G | rs76293757 | COL6A1 | 5.351767279 | 11442 |
| chr12:91651581 | C | T | rs146809131 | DCN | 4.100044118 | −74987 |
| chr12:91646459 | C | T | rs183210465 | DCN | 4.514624315 | −69865 |
| chr15:63276177 | A | T | rs737562 | TPM1 | 3.980870992 | −58769 |
| chr6:56778579 | A | G | rs2817577 | DST | 5.617416743 | 41049 |
| chr15:39868557 | G | A | rs8036633 | THBS1 | 5.476476997 | −4723 |
| chrX:81109059 | C | T | rs181107548 | SH3BGRL | 7.564024112 | 651458 |
| chr5:129941585 | A | G | rs139655460 | HINT1 | 4.708539706 | 559364 |
| chr7:75889061 | C | A | rs60576079 | HSPB1 | 5.564654007 | −42929 |
| chrX:81001793 | G | C | rs149127146 | SH3BGRL | 5.066759519 | 544192 |
| chr7:134423699 | C | T | rs6953906 | CALD1 | 4.166683806 | −40686 |
| chr12:92010962 | C | T | rs147948460 | DCN | 5.053697899 | −434368 |
| chr17:1375533 | A | G | rs75255685 | MYO1C | 6.706885286 | 13162 |
| chr12:15043884 | A | C | rs11056230 | MGP | 4.287151471 | −5096 |
| chr9:123948678 | A | T | rs10760155 | GSN | 4.594008513 | −15083 |
| chr5:130124833 | C | T | rs6860701 | HINT1 | 5.13541921 | 376116 |
| chr8:100908757 | G | T | rs80045067 | COX6C | 5.934380684 | −2822 |
| chr5:130444601 | T | A | rs149263801 | HINT1 | 4.604760613 | 56348 |
| chr7:134508044 | G | A | rs4463365 | CALD1 | 4.203383029 | 43659 |
| chr12:6849428 | A | G | rs12372671 | MLF2 | 5.906810574 | 12654 |
| chr2:238791486 | T | C | rs76033373 | RAMP1 | 4.340770859 | 23220 |
| chr12:91713463 | A | G | rs10777300 | DCN | 5.348443678 | −136869 |
| chr10:45141877 | T | A | rs77695278 | CXCL12 | 5.832892531 | −261332 |
| chr2:232575514 | C | A | rs144176786 | PTMA | 5.526252389 | 2279 |
| chr14:69226147 | C | T | rs184748555 | ZFP36L1 | 4.208415486 | 34484 |
| chr1:169098275 | T | C | rs3766045 | ATP1B1 | 5.248740283 | 22347 |
| chr3:41570973 | T | C | rs114513574 | CTNNB1 | 5.882733448 | 329977 |
| chr9:38302009 | C | T | rs10814672 | ALDH1B1 | 5.881552882 | −90690 |
| chr4:88414538 | A | G | rs146365113 | SPARCL1 | 3.911259796 | 36117 |
| chr2:37846392 | G | A | rs6756578 | CDC42EP3 | 5.252788067 | 52950 |
| chr6:56734711 | T | C | rs13195825 | DST | 5.880068019 | −17997 |
| chr2:20240668 | A | C | rs3731673 | LAPTM4A | 4.954291594 | 10721 |
| chr17:76973339 | C | T | rs1113032 | LGALS3BP | 5.164878778 | 2666 |
| chr20:35166290 | G | A | rs148084305 | MYL9 | 5.192993865 | −3632 |
| chr3:123626861 | T | A | rs189853069 | MYLK | 3.750724358 | −23682 |
| chr14:53475284 | T | G | rs8022561 | FERMT2 | 6.641156711 | −57469 |
| chr9:38249878 | C | T | rs144501928 | ALDH1B1 | 6.481489837 | −142821 |
| chr7:134431646 | A | C | rs2347722 | CALD1 | 4.161085727 | −32739 |
| chr6:56523386 | A | G | rs115757438 | DST | 4.773322958 | −15699 |
| chrX:13016527 | T | C | rs12013535 | TMSB4X | 3.58124692 | 23298 |
| chr12:50150021 | C | T | rs181640433 | TMBIM6 | 5.347356498 | 14429 |
| chr5:85829208 | C | T | rs75886205 | COX7C | 4.701389475 | −84550 |
| chr22:45899628 | G | A | rs56187421 | FBLN1 | 5.086501464 | 865 |
| chr18:3284010 | G | A | rs1791109 | MYL12B | 5.057250429 | 21293 |
| chr15:63285756 | A | T | rs188773930 | TPM1 | 3.941103695 | −49190 |
| chr6:32396964 | C | T | rs9268582 | HLA-DRA | 5.93283806 | −10700 |
| chr2:20618921 | C | T | rs6755136 | RHOB | 4.499292802 | −27914 |
| chr6:29912713 | C | T | rs17879496 | HLA-A | 4.102002643 | 2404 |
| chr1:40520607 | C | T | rs61781906 | CAP1 | 5.185469375 | 14084 |
| chrX:12991870 | T | C | rs187584456 | TMSB4X | 4.073157161 | −1359 |
| chr3:123614467 | G | T | rs2700363 | MYLK | 6.692950642 | −11288 |
| chr2:238260281 | C | G | rs3790996 | COL6A3 | 4.388107205 | 62526 |
| chr6:31239681 | A | T | rs9264669 | HLA-C | 5.063488744 | 188 |
| chr6:169044025 | T | C | rs6900667 | SMOC2 | 4.543741457 | 202161 |
| chr5:129877366 | A | G | rs186147439 | HINT1 | 4.792994671 | 623583 |
| chr12:6851649 | G | A | rs10849509 | MLF2 | 7.098856606 | 10433 |
| chr3:57907854 | C | T | rs149976597 | SLMAP | 5.856220769 | 32125 |
| chr8:101864653 | G | A | rs10096785 | YWHAZ | 5.016898025 | 98146 |
| chr10:97166393 | A | G | rs142405424 | SORBS1 | 5.921836048 | 34544 |
| chr10:70081410 | G | T | rs185684202 | HNRNPH3 | 6.932634735 | −10040 |
| chr5:149786925 | G | A | rs78441286 | CD74 | 4.922222234 | 5394 |
| chr5:150547844 | G | A | rs62379703 | ANXA6 | 6.758603752 | −10504 |
| chr11:8844356 | T | C | rs10840132 | ST5 | 6.538162294 | −11474 |
| chr14:69426525 | T | C | rs139915719 | ACTN1 | 4.618423842 | 19494 |
| chr6:56391031 | C | T | rs187893981 | DST | 5.15152805 | 116656 |
| chr4:119911668 | G | T | rs6850935 | SYNPO2 | 5.005855635 | 101659 |
| chr7:134418852 | A | G | rs181926699 | CALD1 | 4.055875111 | −45533 |
| chr6:56508802 | A | G | rs59772940 | DST | 4.655503958 | −1115 |
| chr9:76123611 | C | T | rs117128393 | ANXA1 | 6.597584739 | 356830 |
| chr10:97242901 | G | A | rs4244315 | SORBS1 | 5.324685831 | −41964 |
| chr7:134458696 | C | T | rs1968701 | CALD1 | 4.339921228 | −5689 |
| chr6:118860667 | C | T | rs76862799 | PLN | 6.521354616 | −8792 |
| chr1:40514331 | T | C | rs11207417 | CAP1 | 5.108818229 | 7808 |
| chr1:95378425 | C | G | rs864553 | CNN3 | 5.082287875 | 12939 |
| chr3:41122240 | G | A | rs142010326 | CTNNB1 | 4.886438176 | −118756 |
| chr1:145446285 | T | C | rs704986 | TXNIP | 4.058804335 | 6960 |
| chr3:123626783 | T | A | rs34179702 | MYLK | 4.02426006 | −23604 |
| chr4:186424823 | T | C | rs138329417 | PDLIM3 | 5.121153593 | 31889 |
| chr7:134407360 | C | G | rs2347710 | CALD1 | 3.920797892 | −57025 |
| chrX:102830633 | C | T | rs190241464 | TCEAL4 | 4.198398744 | −526 |
| chr5:130378833 | T | C | rs148554474 | HINT1 | 5.841958856 | 122116 |
| chr10:97332195 | C | T | rs17110984 | SORBS1 | 4.607140012 | −11067 |
| chr10:97214419 | G | A | rs146971392 | SORBS1 | 5.101893861 | −13482 |
| chr2:219078618 | T | C | rs58933582 | ARPC2 | 4.874717072 | −3294 |
| chr22:31456863 | A | G | rs5994368 | SMTN | 5.160249379 | −20441 |
| chr12:91931232 | A | G | rs12314582 | DCN | 4.549644701 | −354638 |
| chr2:216277672 | T | C | rs7588830 | FN1 | 5.914303625 | 23119 |
| chr17:7116853 | C | A | rs446994 | ACADVL | 5.282274783 | −3591 |
| chrX:114705371 | T | C | rs112749728 | PLS3 | 6.404286064 | −90130 |
| chr21:27311354 | G | C | rs139643655 | APP | 5.306227803 | 201354 |
| chr1:192828509 | T | A | rs7542329 | RGS2 | 4.847499126 | 50340 |
| chr6:31246676 | C | T | rs67394384 | HLA-C | 5.289833103 | −6763 |
| chr21:47405598 | A | G | rs73159603 | COL6A1 | 4.038283229 | 3935 |
| chr2:105988915 | G | C | rs56120302 | FHL2 | 6.986215822 | 26593 |
| chr13:48746873 | T | C | rs150387987 | ITM2B | 5.893202117 | −60469 |
| chr6:56739387 | T | A | rs13216793 | DST | 5.49667122 | −22673 |
| chr11:70213182 | C | T | rs7941965 | CTTN | 5.197093162 | −31430 |
| chr7:99737164 | G | A | rs56733782 | LAMTOR4 | 6.203343328 | −9377 |
| chr7:116154783 | T | C | rs7801950 | CAV1 | 4.666453712 | −10280 |
| chr4:108909446 | A | G | rs72890536 | HADH | 5.484326649 | −1424 |
| chr2:20309589 | T | G | rs192893860 | LAPTM4A | 4.861317603 | −58200 |
| chr20:17555957 | G | C | rs117049909 | DSTN | 3.752252578 | 5237 |
| chr4:10199139 | T | G | rs11726996 | WDR1 | 6.059297062 | −80716 |
| chr22:33178745 | T | C | rs4359739 | TIMP3 | 4.647948444 | −18057 |
| chr10:44889134 | C | G | rs77456211 | CXCL12 | 3.996804622 | −8589 |
| chr1:40520019 | C | T | rs35017768 | CAP1 | 5.058982298 | 13496 |
| chr14:103943140 | T | C | rs17617832 | CKB | 5.358249955 | 46027 |
| chr19:39130846 | A | G | rs144345280 | ACTN4 | 5.052680634 | −7443 |
| chr2:189821670 | A | T | rs7585650 | COL3A1 | 3.672490676 | −17429 |
| chr6:29912564 | A | C | rs17885126 | HLA-A | 4.089801516 | 2255 |
| chr1:115291978 | C | T | rs4140445 | CSDE1 | 4.703514399 | 8627 |
| chr20:36800821 | G | A | rs56388206 | TGM2 | 6.300725155 | −5911 |
| chr11:102295477 | T | C | rs12293834 | TMEM123 | 6.521796305 | 28019 |
| chr10:79530263 | T | C | rs78464275 | KCNMA1 | 4.777148962 | −132697 |
| chr2:238781335 | C | G | rs142344557 | RAMP1 | 4.799806401 | 13069 |
| chr4:58004310 | C | A | rs11945795 | IGFBP7 | 4.113631188 | −27759 |
| chr1:203609879 | A | G | rs41447445 | ATP2B4 | 5.11934689 | 12814 |
| chr11:12439472 | T | C | rs144379817 | PARVA | 6.629435159 | 40354 |
| chr5:151073643 | T | A | rs6579890 | SPARC | 3.914434612 | −7167 |
| chr10:30162660 | G | A | rs10826718 | SVIL | 4.851647243 | −136795 |
| chr18:3263819 | G | A | rs1791061 | MYL12B | 4.811352752 | 1102 |
| chrX:153073319 | A | G | rs58306331 | SSR4 | 5.378379896 | 13196 |
| chr6:29921779 | C | T | rs9260557 | HLA-A | 4.875484556 | 11470 |
| chr12:9290862 | G | A | rs2377740 | A2M | 4.129127557 | −22037 |
| chr7:101459147 | T | C | rs190979222 | CUX1 | 6.727375165 | −140 |
| chr12:65802055 | G | A | rs11175753 | MSRB3 | 5.035038684 | 129288 |
| chr4:88471517 | A | G | rs74397971 | SPARCL1 | 4.433149397 | −20862 |
| chr21:27636112 | T | C | rs143501154 | APP | 4.506368281 | −92666 |
| chr6:31228460 | A | G | rs9264368 | HLA-C | 5.463274643 | 11409 |
| chr6:122791469 | C | T | rs75254937 | SERINC1 | 5.246874939 | 1483 |
| chr2:9823674 | C | T | rs4669416 | YWHAQ | 5.319089182 | −52548 |
| chr5:172222943 | G | T | rs115698579 | DUSP1 | 4.290238637 | −24745 |
| chr2:74135898 | A | G | rs756128 | ACTG2 | 3.35311614 | 15763 |
| chr6:169001953 | T | C | rs143667196 | SMOC2 | 10.22422291 | 160089 |
| chr21:27530922 | A | G | rs12233316 | APP | 4.649955332 | 12166 |
| chr5:130124134 | C | A | rs147509492 | HINT1 | 6.017828368 | 376815 |
| chr19:11310106 | C | T | rs78942291 | KANK2 | 5.842450373 | −1885 |
| chr2:238817063 | A | G | rs59720286 | RAMP1 | 7.30206118 | 48797 |
| chr8:109255206 | T | C | rs629996 | EIF3E | 5.023502124 | 5740 |
| chr3:41562478 | A | T | rs140973429 | CTNNB1 | 6.826321232 | 321482 |
| chr14:53369830 | C | A | rs75278020 | FERMT2 | 4.881839552 | 47938 |
| chr6:169001825 | G | A | rs146535396 | SMOC2 | 7.414687946 | 159961 |
| chr4:10284455 | C | G | rs74916829 | WDR1 | 4.536182173 | −166032 |
| chr6:44212496 | T | C | rs324129 | HSP90AB1 | 4.422130175 | −1407 |
| chr4:1178810 | T | A | rs150779200 | SPON2 | 4.554858916 | −11811 |
| chr19:11286705 | G | A | rs11880059 | KANK2 | 5.888550617 | 18561 |
| chr1:169176469 | G | A | rs1080267 | ATP1B1 | 4.968879372 | 100541 |
| chr4:58102553 | A | G | rs9999844 | IGFBP7 | 8.817228843 | −126002 |
| chr2:238787247 | C | T | rs13405506 | RAMP1 | 5.309814662 | 18981 |
| chr4:169809156 | G | A | rs114635829 | PALLD | 4.153305851 | 55910 |
| chr2:219084082 | T | C | rs1514132 | ARPC2 | 4.667672738 | 1962 |
| chr14:52005924 | C | T | rs11844403 | FRMD6 | 8.621776849 | 50078 |
| chr4:10235742 | C | T | rs184155711 | WDR1 | 5.908283889 | −117319 |
| chr14:103967894 | G | C | rs76665290 | CKB | 4.327520765 | 21273 |
| chr18:12316779 | C | T | rs12607321 | TUBB6 | 9.548217711 | 8539 |
| chr1:192788379 | G | C | rs17647548 | RGS2 | 5.037457359 | 10210 |
| chr5:130337870 | G | A | rs74576088 | HINT1 | 5.68723565 | 163079 |
| chr3:123580416 | T | A | rs146298511 | MYLK | 3.662729525 | 22763 |
| chr17:79461768 | C | G | rs7213703 | ACTG1 | 9.138789736 | 18057 |
| chr6:31337654 | G | A | rs2523530 | HLA-B | 5.611688204 | −12698 |
| chr2:20274446 | C | T | rs11685443 | LAPTM4A | 4.709017426 | −23057 |
| chr4:169630204 | T | G | rs58395080 | PALLD | 4.40101726 | 77475 |
| chr6:56516853 | A | T | rs74940960 | DST | 4.571589972 | −9166 |
| chr7:134448442 | A | G | rs111729270 | CALD1 | 3.94026693 | −15943 |
| chr5:151088967 | C | A | rs73794307 | SPARC | 4.501708001 | −22491 |
| chr6:29923314 | C | T | rs9260635 | HLA-A | 5.908670425 | 13005 |
| chr21:47500830 | A | T | rs61042748 | COL6A2 | 4.283882491 | −17196 |
| chr3:123564698 | C | T | rs7634556 | MYLK | 3.873222397 | 38481 |
| chr15:39751729 | A | G | rs7171447 | THBS1 | 4.941100331 | −121551 |
| chr4:169471808 | C | T | rs7678734 | PALLD | 5.129082433 | 53605 |
| chr4:88425107 | T | G | rs115272416 | SPARCL1 | 3.699372369 | 25548 |
| chr5:179255210 | C | T | rs144700075 | SQSTM1 | 5.43094415 | 7305 |
| chr4:119905430 | C | T | rs75585432 | SYNPO2 | 4.645557555 | 95421 |
| chr1:183583950 | C | T | rs78070601 | ARPC5 | 5.542105101 | 20968 |
| chr6:31325092 | A | G | rs9266217 | HLA-B | 5.616413765 | −136 |
| chr16:15939935 | G | A | rs215577 | MYH11 | 4.123689798 | 10950 |
| chr4:119915395 | G | A | rs6815775 | SYNPO2 | 4.819384217 | 105386 |
| chr12:21819702 | T | A | rs7301883 | LDHB | 5.397187774 | −8913 |
| chr4:82962211 | T | C | rs139548724 | HNRNPD | 6.889627427 | 332933 |
| chr1:95374403 | A | T | rs75703622 | CNN3 | 4.589185584 | 16961 |
| chr7:115877874 | G | A | rs60531573 | TES | 4.503999241 | 14867 |
| chr8:98891518 | C | T | rs80285745 | MATN2 | 7.277906984 | 10226 |
| chr1:59240624 | G | A | rs186636348 | JUN | 4.204241671 | 9095 |
| chr9:123941033 | G | C | rs2146837 | GSN | 4.446646259 | −22728 |
| chr14:103981601 | A | G | rs2023397 | CKB | 6.206693985 | 7566 |
| chr10:73640555 | T | A | rs11000046 | PSAP | 4.092758521 | −29547 |
| chr6:29922932 | T | C | rs9260610 | HLA-A | 4.393507355 | 12623 |
| chr15:63305332 | G | T | rs12909667 | TPM1 | 4.038616884 | −29614 |
| chr21:41326036 | C | T | rs111306421 | PCP4 | 4.137521684 | 86672 |
| chr1:202456278 | A | G | rs705739 | PPP1R12B | 5.054572418 | 24436 |
| chr21:27514373 | A | G | rs375369 | APP | 5.535105355 | −1665 |
| chr6:29907740 | G | A | rs9260043 | HLA-A | 5.196044743 | −2507 |
| chr6:29917881 | T | C | rs2517714 | HLA-A | 4.120626878 | 7572 |
| chr14:23058196 | C | A | rs5742730 | DAD1 | 5.014856234 | −66 |
| chr6:139662346 | A | G | rs76057840 | CITED2 | 4.587496306 | 33004 |
| chrX:12995343 | G | C | rs35474586 | TMSB4X | 3.523171749 | 2114 |
| chr1:156106982 | G | A | rs201583907 | LMNA | 5.392460488 | 11021 |
| chr2:238236921 | C | T | rs6720773 | COL6A3 | 6.212432161 | 85886 |
| chr10:97137105 | A | G | rs1536556 | SORBS1 | 7.055637436 | 63832 |
| chr5:85886752 | C | T | rs149671164 | COX7C | 4.896589778 | −27006 |
| chr4:88448026 | A | C | rs2011326 | SPARCL1 | 4.501120467 | 2629 |
| chr3:112387074 | G | A | rs61418949 | CCDC80 | 4.177924972 | −27084 |
| chr17:66373186 | G | A | rs12453579 | PRKAR1A | 5.738502145 | −36578 |
| chr10:45137149 | A | G | rs2046090 | CXCL12 | 4.598162957 | −256604 |
| chr19:39181778 | G | A | rs7258742 | ACTN4 | 5.690152645 | 43489 |
| chr21:41333849 | C | T | rs16999058 | PCP4 | 5.199766317 | 94485 |
| chr7:852039 | C | T | rs75971508 | SUN1 | 5.335368328 | −3155 |
| chr4:82905273 | A | G | rs138592360 | HNRNPD | 5.699786991 | 389871 |
| chr1:40507168 | A | G | rs34619898 | CAP1 | 4.869220778 | 645 |
| chr4:80942772 | T | C | rs10857135 | ANTXR2 | 6.369851352 | 51611 |
| chr12:91745885 | G | A | rs17251176 | DCN | 3.858414343 | −169291 |
| chr10:75809794 | C | G | rs11596000 | VCL | 5.1215374 | 51920 |
| chr9:124004471 | A | G | rs12343145 | GSN | 3.886588402 | −25969 |
| chr7:116547816 | C | G | rs187982195 | CAPZA2 | 8.6755255 | 45175 |
| chr1:19800116 | G | C | rs78136751 | CAPZB | 5.597017519 | 10673 |
| chr3:123345911 | T | C | rs41271431 | MYLK | 3.567428887 | −6516 |
| chr5:40979106 | T | C | rs111724414 | C7 | 5.286057219 | 69507 |
| chr20:4586382 | T | G | rs78812537 | PRNP | 5.88071987 | −80720 |
| chr9:124063630 | T | C | rs112532363 | GSN | 4.367966056 | 1521 |
| chr1:59241988 | C | T | rs34451092 | JUN | 4.535130337 | 7731 |
| chr21:41261453 | A | T | rs56209274 | PCP4 | 5.279741753 | 22089 |
| chr5:130411584 | C | T | rs114138966 | HINT1 | 4.449405894 | 89365 |
| chr3:50087947 | A | G | rs144406288 | RBM5 | 6.812204092 | −38405 |
| chr4:58005656 | G | A | rs73242662 | IGFBP7 | 3.768848716 | −29105 |
| chr21:47484153 | A | G | rs113930276 | COL6A2 | 4.224356636 | −33873 |
| chr10:30125270 | C | T | rs72806924 | SVIL | 8.377890624 | −99405 |
| chr1:192783297 | A | G | rs141129523 | RGS2 | 5.221367786 | 5128 |
| chr20:44551855 | C | T | rs6073958 | PLTP | 7.643800857 | −10852 |
| chr4:108938840 | A | G | rs10008953 | HADH | 5.18316444 | 13020 |
| chr22:33129556 | G | A | rs147811322 | TIMP3 | 5.868609324 | −67246 |
| chr3:152011385 | G | C | rs115539560 | MBNL1 | 6.250741908 | −5418 |
| chr12:91723761 | A | G | rs116196989 | DCN | 5.208566589 | −147167 |
| chr2:220288963 | G | A | rs6759920 | DES | 6.706252811 | 5864 |
| chr8:117759057 | C | T | rs80057702 | EIF3H | 4.653669972 | 9005 |
| chr1:202504183 | A | G | rs12127239 | PPP1R12B | 5.412946142 | −5059 |
| chr1:164375057 | A | G | rs147510697 | PBX1 | 6.074969654 | −153540 |
| chr11:100972889 | A | T | rs117732947 | PGR | 5.303328307 | 26905 |
| chr15:39726950 | A | G | rs139207500 | THBS1 | 7.039140575 | −146330 |
| chr10:30162275 | T | C | rs72808907 | SVIL | 4.405710176 | −136410 |
| chr7:154038006 | T | C | rs6464412 | DPP6 | 7.853591962 | 35762 |
| chr12:21818267 | T | A | rs7297042 | LDHB | 6.632933635 | −7478 |
| chr1:173474429 | T | C | rs12026637 | PRDX6 | 4.91047679 | 27955 |
| chr12:13379036 | G | C | rs139083777 | EMP1 | 6.454780996 | 29376 |
| chr11:332857 | A | C | rs7937786 | IFITM3 | 5.339585152 | −11997 |
| chr10:79086859 | G | A | rs10509385 | KCNMA1 | 4.538826579 | 23660 |
| chr6:31203779 | G | A | rs141444295 | HLA-C | 4.954389352 | 36090 |
| chr5:138275068 | G | A | rs189972151 | CTNNA1 | 5.854563825 | 63979 |
| chr2:106004255 | G | A | rs35920582 | FHL2 | 5.027368832 | 11253 |
| chr6:118905991 | A | G | rs12192306 | PLN | 5.295738876 | 36532 |
| chr11:102299343 | C | A | rs7951999 | TMEM123 | 5.276490711 | 24153 |
| chr14:69216525 | G | A | rs141491931 | ZFP36L1 | 4.782330878 | 44106 |
| chr7:5594385 | T | C | rs852536 | ACTB | 4.03522335 | −24153 |
| chr6:109598551 | T | C | rs185030778 | CD164 | 5.659364322 | 104391 |
| chr8:38633335 | A | T | rs59836717 | TACC1 | 7.275627996 | −11419 |
| chr6:31345064 | C | T | rs7754026 | HLA-B | 4.158528503 | −20108 |
| chr9:38298057 | A | G | rs10973735 | ALDH1B1 | 5.680077802 | −94642 |
| chr4:10134448 | G | C | rs1109472 | WDR1 | 5.155718029 | −16025 |
| chr4:80912555 | G | T | rs34859312 | ANTXR2 | 4.919177893 | 81828 |
| chr9:90987676 | G | A | rs187003331 | SPIN1 | 7.096481426 | −15683 |
| chr6:168926519 | C | T | rs73041929 | SMOC2 | 5.651264023 | 84655 |
| chr17:66371197 | A | G | rs2160724 | PRKAR1A | 6.35673847 | −38567 |
| chr12:91636476 | C | T | rs182253293 | DCN | 6.49913081 | −59882 |
| chr16:15898999 | G | A | rs7185312 | MYH11 | 3.44849919 | 51886 |
| chr10:97328145 | G | A | rs7072291 | SORBS1 | 4.422593415 | −7017 |
| chrX:81141693 | T | C | rs146133274 | SH3BGRL | 5.684190186 | 684092 |
| chr14:102547203 | A | G | rs189791627 | HSP90AA1 | 4.793060161 | 6178 |
| chr7:46080834 | T | G | rs80001023 | IGFBP3 | 7.585405 | −119963 |
| chr11:46402988 | C | T | rs111790356 | MDK | 5.819002092 | −231 |
| chr6:56509842 | T | C | rs139060735 | DST | 4.498778358 | −2155 |
| chr10:97348725 | T | C | rs11188382 | SORBS1 | 4.585455507 | −27597 |
| chr2:85124047 | C | T | rs13406430 | TMSB10 | 4.455665397 | −8733 |
| chr6:44214869 | T | A | rs35074133 | HSP90AB1 | 8.806155934 | 31 |
| chr11:100933412 | A | C | rs1042838 | PGR | 5.65219569 | 66382 |
| chr15:64471581 | A | C | rs61143016 | PPIB | 4.12134178 | −16227 |
| chr15:63240237 | C | A | rs72755564 | TPM1 | 4.64641219 | −94709 |
| chr15:64466437 | C | T | rs146632402 | PPIB | 4.857687764 | −11083 |
| chr10:79371139 | C | T | rs2720010 | KCNMA1 | 4.455916882 | 26427 |
| chr21:41206453 | A | G | rs111600775 | PCP4 | 5.344739705 | −32911 |
| chr1:19729217 | C | T | rs75715454 | CAPZB | 5.127918962 | 81572 |
| chr6:29910286 | T | C | rs2916801 | HLA-A | 5.94151983 | −23 |
| chr2:238781006 | G | A | rs74956598 | RAMP1 | 5.594214797 | 12740 |
| chr9:113057369 | A | C | rs13298015 | TXN | 5.239843884 | −38582 |
| chr6:132648726 | T | C | rs77286333 | MOXD1 | 6.240198333 | 73888 |
| chr3:112370669 | G | A | rs16859911 | CCDC80 | 4.889395647 | −10679 |
| chr12:91519477 | A | G | rs78076195 | LUM | 4.396824166 | −14206 |
| chr6:168927211 | G | T | rs112409103 | SMOC2 | 5.923485439 | 85347 |
| chr1:153479905 | A | G | rs28632016 | S100A6 | 5.40376693 | 28562 |
| chr12:21853012 | G | A | rs11046166 | LDHB | 4.731072838 | −42223 |
| chr3:112392281 | G | T | rs1249909 | CCDC80 | 4.141364357 | −32291 |
| chr9:75894367 | A | C | rs10869246 | ANXA1 | 5.698343969 | 127586 |
| chr1:173487793 | T | C | rs12035105 | PRDX6 | 4.804878244 | 41319 |
| chr19:16190912 | G | A | rs17641735 | TPM4 | 5.31769054 | 3086 |
| chr7:5574532 | C | G | rs73332425 | ACTB | 3.453926975 | −4300 |
| chr12:118576610 | C | T | rs7977921 | PEBP1 | 5.065791532 | 2681 |
| chr1:202356174 | C | T | rs4950848 | PPP1R12B | 4.973608316 | 38344 |
| chr21:46820936 | G | A | rs8128939 | COL18A1 | 5.519363135 | −4144 |
| chr7:46126597 | T | A | rs139314367 | IGFBP3 | 5.946275543 | −165726 |
| chr1:169165516 | G | A | rs9988604 | ATP1B1 | 5.456150925 | 89588 |
| chr8:101930496 | A | C | rs6983989 | YWHAZ | 4.955172874 | 32303 |
| chr21:41355428 | C | T | rs147693567 | PCP4 | 4.237292651 | 116064 |
| chr11:485552 | C | T | rs111965149 | RNH1 | 7.21775909 | 21216 |
| chr7:22869676 | A | C | rs71520387 | TOMM7 | 4.974691719 | −7208 |
| chr1:19809758 | C | A | rs10917461 | CAPZB | 5.169839198 | 1031 |
| chr14:69187206 | T | A | rs12147980 | ZFP36L1 | 4.297289029 | 73425 |
| chr8:38613641 | G | T | rs59960370 | TACC1 | 6.121070832 | −1152 |
| chr2:238264182 | C | A | rs72986130 | COL6A3 | 6.257415824 | 58625 |
| chr9:92178487 | C | T | rs2183298 | GADD45G | 7.546287253 | −41440 |
| chr10:45103362 | A | G | rs994179 | CXCL12 | 7.333185337 | 222817 |
| chr9:90994996 | C | T | rs188220469 | SPIN1 | 6.595707648 | −8363 |
| chr5:179048205 | C | T | rs4701144 | HNRNPH1 | 4.941778537 | 2197 |
| chr1:202489533 | C | T | rs3827480 | PPP1R12B | 5.097482987 | −19709 |
| chr2:189750376 | T | C | rs35379657 | COL3A1 | 6.003672052 | −88723 |
| chr10:30032160 | T | C | rs10826686 | SVIL | 4.324155152 | −6295 |
| chr17:38617958 | T | C | rs79010101 | IGFBP4 | 4.633372137 | 18256 |
| chr19:49470200 | A | G | rs55945473 | FTL | 3.640060984 | 1634 |
| chr4:169816369 | C | T | rs192083368 | PALLD | 5.453295074 | 63123 |
| chr12:125379948 | C | G | rs61944578 | UBC | 4.049789682 | 19248 |
| chr4:108945631 | C | T | rs3796992 | HADH | 5.032518434 | 19811 |
| chr12:91603198 | C | T | rs181337568 | DCN | 4.089786476 | −26604 |
| chr9:76048231 | G | A | rs116870084 | ANXA1 | 4.499762829 | 281450 |
| chr6:169067445 | T | C | rs111876323 | SMOC2 | 4.451996147 | 225581 |
| chr12:15019987 | C | T | rs142658192 | MGP | 4.03350452 | 18801 |
| chr1:192759702 | A | T | rs12755351 | RGS2 | 4.708839666 | −18467 |
| chr11:119376546 | G | C | rs113269705 | THY1 | 4.791644905 | −80851 |
| chr6:56404054 | C | T | rs184506614 | DST | 4.417966008 | 103633 |
| chr4:10277792 | C | G | rs6836916 | WDR1 | 4.684050432 | −159369 |
| chr2:47437224 | G | A | rs1874669 | CALM2 | 4.126903303 | −33149 |
| chr6:29926597 | C | T | rs1632994 | HLA-A | 4.723002464 | 16288 |
| chr7:46120720 | T | C | rs788714 | IGFBP3 | 5.535971443 | −159849 |
| chr4:169680623 | A | T | rs10023690 | PALLD | 4.154758188 | −72623 |
| chr8:101909258 | C | G | rs4734015 | YWHAZ | 4.627194926 | 53541 |
| chr9:92193148 | A | G | rs11265821 | GADD45G | 7.283469523 | −26779 |
| chr15:39782736 | G | C | rs16969104 | THBS1 | 4.421431739 | −90544 |
| chr1:150551295 | C | T | rs35661734 | MCL1 | 9.269023165 | 791 |
| chr9:102038460 | A | G | rs138503350 | SEC61B | 4.680241346 | 53896 |
| chr9:127992875 | C | T | rs35704262 | HSPA5 | 5.365229845 | 10747 |
| chr3:73309896 | A | G | rs1873006 | PDZRN3 | 7.865917608 | 173250 |
| chr12:15052121 | T | G | rs77528156 | MGP | 4.82393346 | −13333 |
| chr4:88448763 | G | A | rs1381630 | SPARCL1 | 3.626606752 | 1892 |
| chr7:46128249 | G | A | rs1723929 | IGFBP3 | 5.507968813 | −167378 |
| chr15:63277356 | T | C | rs719611 | TPM1 | 4.056814578 | −57590 |
| chr4:88416223 | A | G | rs8342 | SPARCL1 | 3.639781893 | 34432 |
| chr6:31334852 | G | T | rs6457401 | HLA-B | 4.053909928 | −9896 |
| chr4:10273549 | G | A | rs10489071 | WDR1 | 4.552322477 | −155126 |
| chr11:82537924 | A | G | rs12272170 | PRCP | 7.121075682 | 73548 |
| chr10:73607306 | A | G | rs76273335 | PSAP | 4.251245204 | 3702 |
| chr2:217608305 | G | A | rs6751211 | IGFBP5 | 6.894167981 | −48033 |
| chr5:169807717 | C | T | rs111894562 | KCNMB1 | 4.492598884 | 8654 |
| chr19:41115614 | C | T | rs55856492 | LTBP4 | 5.962897576 | 8339 |
| chr1:40530814 | A | G | rs16826849 | CAP1 | 4.711170644 | 24291 |
| chr5:130323746 | G | C | rs139201937 | HINT1 | 5.212673731 | 177203 |
| chr14:75702560 | T | C | rs11627227 | FOS | 4.697057195 | −42971 |
| chr11:32665084 | C | T | rs1324397 | EIF3M | 5.609532557 | 59707 |
| chr6:139688738 | G | A | rs144307071 | CITED2 | 4.541673956 | 6612 |
| chrX:80843232 | A | G | rs189068415 | SH3BGRL | 4.201141265 | 385631 |
| chr5:150558797 | A | G | rs74546280 | ANXA6 | 4.796823057 | −21457 |
| chr22:36777765 | T | G | rs111747416 | MYH9 | 4.453891739 | 6347 |
| chr16:15946778 | T | C | rs9922682 | MYH11 | 3.613839969 | 4107 |
| chr2:238256194 | C | T | rs72986103 | COL6A3 | 6.383623295 | 66613 |
| chr19:39152743 | C | G | rs41482151 | ACTN4 | 4.724595994 | 14454 |
| chr22:36815482 | G | T | rs77952465 | MYH9 | 4.614251256 | −31370 |
| chr6:168971045 | C | G | rs6455533 | SMOC2 | 4.34084028 | 129181 |
| chr4:186453271 | A | G | rs28723051 | PDLIM3 | 5.034151152 | 3441 |
| chr8:109240473 | A | T | rs57222183 | EIF3E | 6.016306043 | 20473 |
| chr10:97320288 | G | A | rs12768682 | SORBS1 | 4.353277003 | 840 |
| chr10:97224435 | A | T | rs7912439 | SORBS1 | 4.725085765 | −23498 |
| chr9:38394344 | C | G | rs12554439 | ALDH1B1 | 5.755774118 | 1645 |
| chr17:38603549 | G | A | rs4890114 | IGFBP4 | 5.931924768 | 3847 |
| chr11:62344743 | C | T | rs117162358 | EEF1G | 6.775425486 | −3380 |
| chr1:32169551 | A | G | rs55775435 | COL16A1 | 5.350018963 | 67 |
| chr11:62281156 | A | G | rs142486301 | AHNAK | 4.934326857 | 31980 |
| chr2:238628107 | A | T | rs56383116 | LRRFIP1 | 6.176249655 | 27259 |
| chr2:33197852 | A | G | rs116009295 | LTBP1 | 6.524929106 | 25832 |
| chr11:101088083 | G | A | rs145433867 | PGR | 5.113200042 | −87539 |
| chr7:134491188 | G | A | rs73724860 | CALD1 | 4.292054549 | 26803 |
| chr17:1246590 | G | A | rs181451348 | YWHAE | 4.66123715 | 56926 |
| chr2:216268902 | T | C | rs7575234 | FN1 | 6.674982519 | 31889 |
| chr4:6630195 | T | C | rs16839049 | MRFAP1 | 6.401377898 | −11623 |
| chr12:6973088 | T | C | rs12824102 | TPI1 | 7.065151259 | −3496 |
| chr5:130375599 | C | A | rs74506654 | HINT1 | 5.037512798 | 125350 |
| chrX:80731653 | G | A | rs186292867 | SH3BGRL | 7.509078029 | 274052 |
| chr6:29918454 | C | T | rs112973287 | HLA-A | 3.979172661 | 8145 |
| chr4:88445893 | C | T | rs61223410 | SPARCL1 | 3.523516357 | 4762 |
| chr12:21819903 | C | T | rs7316475 | LDHB | 5.069515146 | −9114 |
| chr9:38290095 | T | C | rs143961014 | ALDH1B1 | 6.366861016 | −102604 |
| chr6:168817381 | T | C | rs79528632 | SMOC2 | 4.824246503 | −24483 |
| chr10:70049310 | G | A | rs77221173 | HNRNPH3 | 6.747269388 | −42140 |
| chr6:29916468 | T | C | rs3016021 | HLA-A | 3.990616761 | 6159 |
| chr14:24700826 | C | T | rs180729918 | NEDD8 | 5.817703307 | 747 |
| chr12:56567326 | C | T | rs146448375 | MYL6 | 3.895170561 | 15183 |
| chr21:27324875 | G | A | rs2829995 | APP | 4.294944201 | 187833 |
| chr10:97136202 | G | T | rs4918921 | SORBS1 | 4.774687327 | 64735 |
| chr2:54693754 | T | G | rs10169422 | SPTBN1 | 5.846552365 | 10290 |
| chr8:101948681 | G | C | rs3134354 | YWHAZ | 5.429252894 | 14118 |
| chr21:47405449 | C | T | rs760437 | COL6A1 | 5.810316801 | 3786 |
| chr15:39919191 | A | G | rs56722485 | THBS1 | 5.7074768 | 45911 |
| chr12:15027862 | G | A | rs181268794 | MGP | 4.538913965 | 10926 |
| chr7:101519702 | C | T | rs147944713 | CUX1 | 5.465892935 | 58796 |
| chr9:75876977 | G | A | rs17595844 | ANXA1 | 5.547475881 | 110196 |
| chr6:29908789 | C | T | rs112519287 | HLA-A | 4.322248511 | −1458 |
| chr12:98983214 | C | T | rs7964472 | SLC25A3 | 4.556501731 | −4189 |
| chr16:11662869 | T | C | rs56094572 | LITAF | 5.288956165 | 17360 |
| chr2:54837377 | A | G | rs6724136 | SPTBN1 | 6.701083183 | 51846 |
| chr4:108951058 | C | G | rs6832702 | HADH | 4.947792031 | 25238 |
| chr10:30024556 | T | C | rs72631861 | SVIL | 5.848553665 | 174 |
| chr6:29920042 | T | C | rs9260472 | HLA-A | 6.216491137 | 9733 |
| chr22:33132620 | A | C | rs111458941 | TIMP3 | 5.17172778 | −64182 |
| chr15:90976106 | C | T | rs6496673 | IQGAP1 | 6.302995789 | 44632 |
| chr15:97038128 | C | G | rs75638628 | NR2F2 | 6.458758041 | 161134 |
| chr2:231672090 | T | G | rs41489545 | ITM2C | 5.249834019 | −56810 |
| chr11:102085181 | G | A | rs189143339 | YAP1 | 5.560830679 | 102002 |
| chr6:31343981 | C | T | rs2844548 | HLA-B | 5.292921676 | −19025 |
| chr2:47447796 | G | A | rs7559429 | CALM2 | 4.148356952 | −43721 |
| chr14:51966663 | C | T | rs12432710 | FRMD6 | 5.850149207 | 10817 |
| chr2:85137885 | G | A | rs184829440 | TMSB10 | 3.944970984 | 5105 |
| chr17:1695780 | T | A | rs4995138 | SERPINF1 | 4.71855849 | 20815 |
| chrX:23608856 | G | A | rs2136678 | PRDX4 | 7.209744565 | −76754 |
| chr9:76180917 | G | A | rs157122 | ANXA1 | 8.162156236 | 414136 |
| chr15:90805262 | T | C | rs56151832 | NGRN | 5.224816203 | −3633 |
| chr3:123597617 | C | T | rs187544076 | MYLK | 3.902104208 | 5562 |
| chr8:101988845 | G | C | rs2923775 | YWHAZ | 4.390441477 | −23235 |
| chrX:13021430 | C | T | rs12006825 | TMSB4X | 3.438812064 | 28201 |
| chr11:101062126 | C | G | rs186692622 | PGR | 5.76046966 | −61582 |
| chr3:187975926 | G | A | rs6764035 | LPP | 6.203592476 | 32733 |
| chr6:31239752 | C | T | rs9264671 | HLA-C | 4.62362444 | 117 |
| chr11:32610388 | T | A | rs201877 | EIF3M | 5.882666782 | 5011 |
| chr22:36815112 | A | G | rs142756790 | MYH9 | 4.611491486 | −31000 |
| chr1:169159402 | C | A | rs12564558 | ATP1B1 | 4.641573644 | 83474 |
| chr4:10254550 | C | A | rs4697984 | WDR1 | 4.967472274 | −136127 |
| chr1:21005201 | A | G | rs35563952 | DDOST | 6.086442098 | −17164 |
| chr21:41309823 | G | A | rs9978224 | PCP4 | 4.47542062 | 70459 |
| chr17:3569777 | T | C | rs2915545 | TAX1BP3 | 5.30150149 | 2095 |
| chr18:12308793 | C | G | rs11267036 | TUBB6 | 6.504353248 | 553 |
| chr7:134608691 | T | A | rs181840359 | CALD1 | 4.501472394 | 32482 |
| chr3:195294753 | G | T | rs9886989 | APOD | 5.08718729 | 16058 |
| chr8:41225456 | T | G | rs1374585 | SFRP1 | 6.243190025 | −58464 |
| chr6:32418416 | C | T | rs9268763 | HLA-DRA | 4.902090123 | 10752 |
| chr5:130153854 | A | C | rs6895238 | HINT1 | 4.578182995 | 347095 |
| chr9:76353087 | A | G | rs1387289 | ANXA1 | 5.024124143 | 586306 |
| chr11:101140768 | A | T | rs7934843 | PGR | 6.405323315 | −140224 |
| chr3:41420792 | A | C | rs1386601 | CTNNB1 | 5.099039872 | 179796 |
| chr14:52120466 | T | G | rs2357118 | FRMD6 | 6.027693525 | 1838 |
| chr9:123929770 | C | T | rs61745682 | GSN | 4.226955203 | −33991 |
| chr10:105209413 | T | C | rs2232659 | CALHM2 | 7.204723991 | 2714 |
| chr12:7053096 | T | C | rs74057227 | C12orf57 | 4.265436269 | −89 |
| chr11:65617324 | G | A | rs654638 | CFL1 | 5.669794194 | 8327 |
| chr12:50146950 | T | C | rs1012874 | TMBIM6 | 5.396235269 | 11358 |
| chr10:29999266 | A | G | rs4570475 | SVIL | 4.392615557 | 25464 |
| chr15:63276412 | G | A | rs149893785 | TPM1 | 5.376906833 | −58534 |
| chr6:127484959 | T | C | rs189553701 | RSPO3 | 5.467196179 | 45143 |
| chr6:168794380 | T | C | rs9346702 | SMOC2 | 7.01315389 | −47484 |
| chr5:130153723 | G | A | rs143746933 | HINT1 | 5.620303191 | 347226 |
| chr22:36774812 | C | T | rs5756168 | MYH9 | 5.015102093 | 9300 |
| chr5:130351583 | C | G | rs74419571 | HINT1 | 5.282248522 | 149366 |
| chr20:19199747 | T | G | rs138647231 | SLC24A3 | 8.048003505 | 6461 |
| chr4:80905851 | A | C | rs28688624 | ANTXR2 | 5.196823666 | 88532 |
| chr11:101087659 | A | G | rs4121761 | PGR | 5.810863626 | −87115 |
| chrX:23831012 | C | T | rs6653694 | SAT1 | 5.602521896 | 29737 |
| chr8:97866100 | C | T | rs148449390 | CPQ | 6.670843558 | 208630 |
| chr9:102021865 | C | T | rs58028871 | SEC61B | 4.653447643 | 37301 |
| chr1:156644729 | T | C | rs112099817 | NES | 6.931991638 | 2470 |
| chr15:86145311 | A | C | rs12324474 | AKAP13 | 9.549368753 | −17870 |
| chr2:37818236 | C | G | rs2215605 | CDC42EP3 | 4.767217656 | 81106 |
| chr10:45106589 | A | C | rs12242079 | CXCL12 | 3.842539452 | −226044 |
| chr12:50137281 | T | A | rs897056 | TMBIM6 | 5.435886122 | 1689 |
| chr2:217610770 | G | A | rs145990578 | IGFBP5 | 3.586103059 | −50498 |
| chr8:121097603 | T | C | rs10098186 | COL14A1 | 6.668962948 | −39738 |
| chr12:91748428 | C | T | rs114680186 | DCN | 3.54191415 | −171834 |
| chrX:131388718 | A | T | rs5975326 | RAP2C | 5.971897417 | −35210 |
| chr5:150544779 | A | G | rs17801687 | ANXA6 | 5.231501563 | −7439 |
| chr7:99736059 | G | A | rs11771139 | LAMTOR4 | 5.217910931 | −10482 |
| chr4:80904032 | A | G | rs13125598 | ANTXR2 | 4.703308999 | 90351 |
| chr5:150494802 | C | T | rs185738594 | ANXA6 | 5.233719936 | 26443 |
| chr5:169814432 | C | T | rs314109 | KCNMB1 | 4.598193534 | 1939 |
| chr12:109134980 | G | A | rs7959539 | CORO1C | 7.853278136 | −9654 |
| chr16:82489273 | G | A | rs4782644 | CDH13 | 5.996697741 | −171301 |
| chr7:94045576 | G | A | rs42526 | COL1A2 | 4.745425978 | 21703 |
| chr4:80930570 | T | C | rs6534703 | ANTXR2 | 4.594476949 | 63813 |
| chr3:186483598 | G | C | rs9813382 | EIF4A2 | 4.140373319 | −17768 |
| chr5:151093706 | T | C | rs188366049 | SPARC | 4.113998245 | −27230 |
| chr16:15918174 | A | G | rs216159 | MYH11 | 4.052775055 | 32711 |
| chr2:47462712 | G | T | rs6718897 | CALM2 | 4.994065873 | −58637 |
| chr17:39828666 | T | C | rs186038589 | EIF1 | 4.577578185 | −16471 |
| chr17:49263376 | A | G | rs75253709 | NME2 | 5.094893826 | 19293 |
| chr4:16876443 | C | T | rs73799335 | LDB2 | 6.820411682 | 23825 |
| chr12:15027901 | A | T | rs74433861 | MGP | 3.970625531 | 10887 |
| chr1:164361041 | T | A | rs567770 | PBX1 | 7.888325928 | −167556 |
| chr6:169033772 | A | T | rs16887232 | SMOC2 | 4.718759898 | 191908 |
| chr19:13434287 | C | T | rs75123044 | IER2 | 6.467522826 | 173062 |
| chr16:75617065 | G | T | rs73615016 | GABARAPL2 | 10.46002644 | 16788 |
| chr2:54823502 | C | G | rs6739140 | SPTBN1 | 5.676337332 | 37971 |
| chr9:79455225 | G | A | rs10781386 | PRUNE2 | 5.39061293 | 65778 |
| chr7:134499386 | C | A | rs73724864 | CALD1 | 4.069238082 | 35001 |
| chr9:76078277 | C | T | rs184826455 | ANXA1 | 5.944110495 | 311496 |
| chr1:113248365 | G | A | rs954679 | RHOC | 5.468066819 | 1313 |
| chr17:38622868 | C | T | rs72819636 | IGFBP4 | 5.801169463 | 23166 |
| chr19:13297858 | T | C | rs61462951 | IER2 | 6.717856033 | 36633 |
| chr2:64073887 | A | G | rs36099158 | UGP2 | 7.12922674 | 4873 |
| chr2:216298858 | C | T | rs149170829 | FN1 | 4.536524152 | 1933 |
| chr9:127997303 | A | G | rs12009 | HSPA5 | 5.222496279 | 6319 |
| chr20:46503427 | C | T | rs6018765 | SULF2 | 5.407594447 | −88067 |
| chr15:39758749 | T | C | rs11637421 | THBS1 | 4.697160581 | −114531 |
| chr19:13377865 | A | G | rs7249323 | IER2 | 5.341115072 | 116640 |
| chr2:10926819 | C | T | rs73170277 | PDIA6 | 5.3489599 | 26092 |
| chr12:119644265 | G | A | rs111555403 | HSPB8 | 4.972630814 | 27670 |
| chr4:88428298 | A | G | rs12501352 | SPARCL1 | 4.382750634 | 22357 |
| chr8:101943618 | A | G | rs3105452 | YWHAZ | 4.331332128 | 19181 |
| chr11:11982869 | T | C | rs189120268 | DKK3 | 6.431543408 | 47317 |
| chr1:115280782 | C | T | rs41274128 | CSDE1 | 4.904417085 | 19823 |
| chr8:109242803 | A | T | rs674391 | EIF3E | 5.627894457 | 18143 |
| chr20:17582342 | C | T | rs74579742 | DSTN | 7.729892321 | 31622 |
| chr8:125556478 | G | A | rs112168473 | NDUFB9 | 6.646636641 | 5114 |
| chr3:49390651 | C | G | rs183254266 | GPX1 | 6.063300679 | 5135 |
| chr1:39687873 | C | T | rs144971835 | MACF1 | 5.07737048 | 138034 |
| chr19:33056201 | T | C | rs34799080 | PDCD5 | 9.410055829 | −15895 |
| chr1:29526162 | C | T | rs181072609 | SRSF4 | 5.522564724 | −17750 |
| chr1:169087787 | C | T | rs754551 | ATP1B1 | 4.689250663 | 11859 |
| chr8:109347287 | C | T | rs60209465 | EIF3E | 4.582737461 | −86341 |
| chr20:17588916 | G | T | rs34316423 | DSTN | 4.733578954 | 38196 |
| chr10:79541022 | T | C | rs2812439 | KCNMA1 | 4.822707549 | −143456 |
| chr4:10147096 | G | A | rs62287542 | WDR1 | 4.498844668 | −28673 |
| chr1:8937703 | T | G | rs61784221 | ENO1 | 4.230412664 | 1042 |
| chr6:56734722 | A | G | rs6926742 | DST | 5.054601394 | −18008 |
| chr21:47538249 | A | G | rs12483283 | COL6A2 | 4.222095674 | 20216 |
| chr7:137192163 | C | T | rs75306260 | PTN | 5.831146578 | −163684 |
| chr1:202394199 | G | A | rs10158100 | PPP1R12B | 4.815370957 | −37643 |
| chr3:73330625 | C | A | rs492331 | PDZRN3 | 6.549078487 | 152521 |
| chr9:102030173 | G | A | rs78288157 | SEC61B | 4.538451126 | 45609 |
| chr4:186496755 | C | T | rs13109182 | PDLIM3 | 5.742309131 | −40043 |
| chr5:175831645 | C | T | rs7703742 | CLTB | 6.800308377 | 6179 |
| chr5:130176202 | T | G | rs4705896 | HINT1 | 5.246695541 | 324747 |
| chr4:169615229 | T | C | rs6852004 | PALLD | 3.791572491 | 62500 |
| chr9:124078809 | C | T | rs10985204 | GSN | 3.703248762 | 16700 |
| chr20:35131489 | G | C | rs142665323 | MYL9 | 4.757175148 | −38433 |
| chr16:66789125 | A | G | rs11075643 | DYNC1LI2 | 6.075848048 | −3394 |
| chr4:122479213 | C | T | rs139155583 | ANXA5 | 4.825256737 | 138922 |
| chr4:10200204 | G | T | rs55775442 | WDR1 | 4.972473178 | −81781 |
| chr3:120325167 | T | C | rs6789801 | NDUFB4 | 6.270139921 | 9984 |
| chr5:130059868 | G | A | rs113064282 | HINT1 | 4.304767353 | 441081 |
| chr14:103953501 | T | G | rs12878548 | CKB | 5.089226589 | 35666 |
| chr3:123350629 | T | C | rs115655522 | MYLK | 5.25739843 | −11234 |
| chr7:46081941 | T | C | rs148367379 | IGFBP3 | 5.896458334 | −121070 |
| chr14:21712385 | G | T | rs183478003 | HNRNPC | 6.005659625 | 25253 |
| chr6:118885569 | T | C | rs12199508 | PLN | 4.951641187 | 16110 |
| chr1:40518047 | T | C | rs181131334 | CAP1 | 5.032145537 | 11524 |
| chr4:169613806 | C | A | rs57498842 | PALLD | 3.889351803 | 61077 |
| chr7:46310621 | A | G | rs146703969 | IGFBP3 | 6.068028081 | −349750 |
| chr2:20650965 | C | T | rs342054 | RHOB | 4.004582046 | 4130 |
| chr8:98937119 | T | C | rs145996472 | MATN2 | 5.33290524 | 55827 |
| chr21:41269531 | G | T | rs74578577 | PCP4 | 5.248075798 | 30167 |
| chr4:108982375 | C | G | rs6533346 | HADH | 5.17141588 | 56555 |
| chr21:27439835 | A | G | rs9974885 | APP | 4.387628261 | 72873 |
| chr18:12337322 | C | T | rs117096851 | TUBB6 | 5.868983301 | 29082 |
| chr12:109897136 | C | T | rs11612500 | KCTD10 | 5.945751191 | 17969 |
| chr6:169049222 | G | A | rs189008825 | SMOC2 | 6.621274874 | 207358 |
| chr17:76929196 | G | A | rs56960228 | TIMP2 | 4.09880314 | −7727 |
| chr7:134559080 | C | T | rs28674970 | CALD1 | 3.911323249 | −17071 |
| chr17:34319442 | T | C | rs854686 | CCL14 | 4.423909895 | −5677 |
| chr21:47519535 | T | C | rs8133436 | COL6A2 | 4.030839652 | 1502 |
| chr10:30010941 | C | T | rs943692 | SVIL | 4.961824119 | 13789 |
| chr21:27481343 | A | C | rs2830057 | APP | 4.697518842 | 31365 |
| chr8:101827447 | G | A | rs192942840 | PABPC1 | 4.573880271 | −93131 |
| chr12:10852106 | T | C | rs17629 | YBX3 | 6.030915981 | 23816 |
| chr7:154173453 | G | A | rs73163046 | DPP6 | 5.600271197 | 171209 |
| chr10:44951929 | C | T | rs116102038 | CXCL12 | 5.393424481 | −71384 |
| chr6:168975780 | C | A | rs2180349 | SMOC2 | 4.256205708 | 133916 |
| chr1:164622360 | G | T | rs73026099 | PBX1 | 4.690652648 | 93401 |
| chr7:134467452 | C | T | rs3800702 | CALD1 | 3.877831027 | 3067 |
| chr11:85695606 | A | G | rs664050 | PICALM | 6.252558003 | 84520 |
| chr11:46402516 | G | A | rs73466027 | MDK | 4.906249144 | −102 |
| chr12:91508477 | A | T | rs186019016 | LUM | 3.94139352 | −3206 |
| chr4:10169977 | C | T | rs143044167 | WDR1 | 5.413612947 | −51554 |
| chr11:485075 | A | G | rs56088382 | RNH1 | 6.035189498 | 21693 |
| chr15:85945753 | G | A | rs59141118 | AKAP13 | 5.894707625 | 21919 |
| chr20:46474342 | G | A | rs181916116 | SULF2 | 7.13522777 | −58982 |
| chr2:20658784 | T | G | rs2348707 | RHOB | 5.514278002 | 11949 |
| chr16:75482860 | G | A | rs12927562 | CFDP1 | 5.809281431 | −15459 |
| chr15:65010075 | A | G | rs16948203 | OAZ2 | 9.861274734 | −14595 |
| chr20:36793966 | T | C | rs2076382 | TGM2 | 5.190175539 | −294 |
| chr9:37425491 | C | T | rs60473180 | GRHPR | 7.702419069 | 2798 |
| chr1:40463498 | G | A | rs61781337 | CAP1 | 4.467204064 | −42239 |
| chr12:120941379 | C | G | rs117337139 | DYNLL1 | 4.467433699 | 7463 |
| chr16:11662463 | G | A | rs77237901 | LITAF | 5.25853768 | 17766 |
| chr6:31232697 | A | G | rs9264493 | HLA-C | 4.662818518 | 7172 |
| chr12:50134247 | T | G | rs58756176 | TMBIM6 | 5.46685487 | −1093 |
| chr2:216304035 | A | G | rs1250228 | FN1 | 4.462743904 | −3242 |
| chr9:131309558 | G | A | rs138328532 | SPTAN1 | 4.598528296 | −5308 |
| chr6:56599297 | T | C | rs141517639 | DST | 4.189526248 | −91610 |
| chr8:109247691 | T | C | rs615524 | EIF3E | 4.512290657 | 13255 |
| chr20:33095745 | T | C | rs77883048 | DYNLRB1 | 4.733557515 | −8444 |
| chr7:30917714 | C | T | rs113846749 | AQP1 | 5.920745963 | −33595 |
| chrX:117950948 | G | A | rs261680 | ZCCHC12 | 4.988345206 | −6839 |
| chrX:64860974 | A | C | rs182321150 | MSN | 6.578384279 | −26547 |
| chr2:55150883 | C | T | rs4671995 | RTN4 | 7.329933219 | 86525 |
| chr6:31327624 | G | A | rs74762609 | HLA-B | 3.966963131 | −2668 |
| chr21:27620433 | A | G | rs73351357 | APP | 4.098033008 | −76987 |
| chr11:101065007 | C | T | rs4595509 | PGR | 6.285909031 | −64463 |
| chr6:31249553 | C | T | rs191706767 | HLA-C | 4.668607935 | −9640 |
| chr6:169029503 | C | T | rs16887223 | SMOC2 | 4.694470316 | 187639 |
| chr16:15915820 | G | A | rs216152 | MYH11 | 4.391511604 | 35065 |
| chr7:115767716 | G | A | rs56038916 | TES | 4.35737919 | −82877 |
| chr4:10210648 | A | C | rs185700151 | WDR1 | 4.882924047 | −92225 |
| chr5:72827129 | T | C | rs959179 | BTF3 | 4.355283277 | 32879 |
| chr6:31339844 | G | A | rs2523525 | HLA-B | 4.7687285 | −14888 |
| chr4:88929138 | C | T | i5047408 | PKD2 | 7.350904197 | 351 |
| chr20:48177341 | G | A | rs139610481 | PTGIS | 7.804044271 | 7333 |
| chr2:189823217 | A | G | rs34139968 | COL3A1 | 3.506566263 | −15882 |
| chr7:6531375 | G | A | rs111283052 | KDELR2 | 5.751402043 | −7592 |
| chr8:109235457 | A | G | rs635941 | EIF3E | 5.966691268 | 25489 |
| chr3:128404269 | A | G | rs11712115 | RPN1 | 6.927942894 | −34608 |
| chr2:238556404 | A | G | rs80315294 | LRRFIP1 | 7.380836643 | 20174 |
| chr11:74375488 | G | T | rs186792395 | CHRDL2 | 5.307426863 | 46924 |
| chr22:36803750 | T | A | rs113916029 | MYH9 | 4.303337014 | −19638 |
| chr9:76184436 | G | A | rs157117 | ANXA1 | 6.291622333 | 417655 |
| chr9:75902188 | C | A | rs10117461 | ANXA1 | 8.316284494 | 135407 |
| chr5:151077328 | A | G | rs9324706 | SPARC | 5.242047012 | −10852 |
| chr13:107214886 | C | T | rs9514550 | ARGLU1 | 6.112256963 | 5599 |
| chr7:134418334 | C | A | rs12707171 | CALD1 | 3.87707616 | −46051 |
| chr2:238281828 | T | C | rs79058172 | COL6A3 | 6.276173331 | 40979 |
| chr20:36798627 | G | A | rs11698249 | TGM2 | 5.196243115 | −3717 |
| chr14:55616937 | T | G | rs2340931 | LGALS3 | 5.621487265 | 13236 |
| chr14:69193230 | C | T | rs61984984 | ZFP36L1 | 4.115769542 | 67401 |
| chr7:137113526 | A | C | rs185072315 | PTN | 4.864379871 | −85047 |
| chr12:21841978 | A | T | rs10841880 | LDHB | 4.50923725 | −31189 |
| chr15:55436087 | C | G | rs185045872 | RSL24D1 | 6.569302846 | 53052 |
| chr2:20620275 | T | C | rs11673928 | RHOB | 4.370752085 | −26560 |
| chr4:103778091 | C | T | rs148956604 | UBE2D3 | 6.421051063 | 11959 |
| chr17:15197417 | G | T | rs4792586 | PMP22 | 4.518365855 | −28774 |
| chr9:38342834 | T | C | rs35230605 | ALDH1B1 | 5.050555132 | −49865 |
| chr4:122633402 | G | A | rs142654842 | ANXA5 | 5.971338823 | −15267 |
| chr4:122478727 | A | G | rs4833747 | ANXA5 | 5.811660811 | 139408 |
| chr15:30201173 | C | T | rs190618973 | TJP1 | 5.63558571 | 60079 |
| chr9:75686239 | G | A | rs187228020 | ANXA1 | 5.313767511 | −80542 |
| chr1:203625905 | C | G | rs188180667 | ATP2B4 | 4.007253039 | 28840 |
| chr4:57983838 | T | A | rs141474657 | IGFBP7 | 3.864960391 | −7287 |
| chr10:79531935 | C | G | rs16935355 | KCNMA1 | 4.346188977 | −134369 |
| chr1:154608418 | T | G | rs11264230 | ADAR | 7.848474905 | −7945 |
| chr11:325948 | C | T | rs10794309 | IFITM3 | 3.819691129 | −5088 |
| chr4:82900876 | A | C | rs111266598 | HNRNPD | 5.110494343 | 394268 |
| chr6:56538327 | T | C | rs73746929 | DST | 4.174361983 | −30640 |
| chr7:115782546 | T | C | rs4727825 | TES | 5.39011579 | −68047 |
| chr6:168924891 | G | A | rs12200380 | SMOC2 | 5.464089494 | 83027 |
| chr9:102006819 | C | T | rs17725679 | SEC61B | 4.922546409 | 22255 |
| chr11:32651024 | G | A | rs400655 | EIF3M | 5.181634687 | 45647 |
| chr2:9785377 | C | A | rs73913152 | YWHAQ | 4.74372521 | −14251 |
| chr19:9937271 | T | A | rs12461515 | UBL5 | 4.120369963 | −1323 |
| chr12:65808584 | A | G | rs11175755 | MSRB3 | 7.039056393 | 135817 |
| chr15:63270947 | C | A | rs10851719 | TPM1 | 3.825799748 | −63999 |
| chr4:95369673 | A | G | rs145894951 | PDLIM5 | 4.685390173 | −3335 |
| chr3:123564818 | C | T | rs7634655 | MYLK | 3.655772639 | 38361 |
| chr3:48100395 | A | G | rs140371849 | MAP4 | 5.101225683 | 29943 |
| chr1:192829056 | A | C | rs144859249 | RGS2 | 4.880140703 | 50887 |
| chr5:10944130 | G | A | rs1995363 | DAP | 5.90528816 | −182784 |
| chr12:98967005 | T | C | rs34261110 | SLC25A3 | 4.519070787 | −20398 |
| chr11:117061197 | C | T | rs143668212 | TAGLN | 3.289767094 | −8813 |
| chr13:48794101 | C | T | rs9595871 | ITM2B | 4.010573253 | −13241 |
| chr5:149807964 | A | G | rs79818596 | CD74 | 3.800288914 | −15465 |
| chr2:54844995 | G | A | rs7608414 | SPTBN1 | 5.34848363 | 59464 |
| chr2:231668177 | G | A | rs143781938 | ITM2C | 6.369923055 | −60723 |
| chr15:86132752 | T | C | rs62023931 | AKAP13 | 6.402389817 | −30429 |
| chr9:131303036 | T | C | rs79716800 | SPTAN1 | 4.644304477 | −11830 |
| chr6:7854284 | A | G | rs58106097 | TXNDC5 | 6.40997001 | 56026 |
| chr11:32682327 | T | C | rs79851266 | EIF3M | 5.132672042 | 76950 |
| chr5:129877472 | G | A | rs10074110 | HINT1 | 6.3163638 | 623477 |
| chr4:169635972 | G | A | rs72699852 | PALLD | 4.607567157 | 83243 |
| chr4:169702077 | A | G | rs7656866 | PALLD | 4.08997355 | −51169 |
| chr20:17556930 | C | T | rs73258639 | DSTN | 4.001498818 | 6210 |
| chr11:117764641 | G | A | rs11216607 | FXYD6 | 4.076263676 | −16496 |
| chr19:45983527 | T | C | rs73567081 | FOSB | 5.070022494 | 12273 |
| chr2:238241450 | C | T | rs56033985 | COL6A3 | 4.158008713 | 81357 |
| chr1:212740145 | C | G | rs685330 | ATF3 | 4.99866955 | 1469 |
| chr10:32423872 | G | T | rs2998025 | KIF5B | 7.614019662 | −78519 |
| chr12:124075921 | T | C | rs66494087 | TMED2 | 5.322067067 | 6822 |
| chr2:219097773 | A | G | rs13388024 | ARPC2 | 4.33130376 | 15653 |
| chr15:90949403 | C | T | rs7175440 | IQGAP1 | 5.417675211 | 17929 |
| chr2:238228356 | G | A | rs11679346 | COL6A3 | 5.432521999 | 94451 |
| chr19:33039726 | A | G | rs12980000 | PDCD5 | 6.330904886 | −32370 |
| chr6:36944891 | G | A | rs150513025 | MTCH1 | 5.144294277 | 9436 |
| chr10:45087000 | G | A | rs140314021 | CXCL12 | 3.755082674 | −206455 |
| chr3:123336982 | G | A | rs848145 | MYLK | 6.75380487 | 2413 |
| chr19:48831264 | T | C | rs45570234 | EMP3 | 6.290421486 | 2008 |
| chr8:101865648 | C | T | rs146288431 | YWHAZ | 4.393726408 | 97151 |
| chr8:95313019 | G | A | rs2445723 | GEM | 5.851737626 | −38472 |
| chr2:20310877 | C | A | rs192202930 | LAPTM4A | 4.053381751 | −59488 |
| chr7:134398093 | A | G | rs73724834 | CALD1 | 5.258573536 | −66292 |
| chr4:10130567 | A | G | rs59368604 | WDR1 | 4.50313136 | −12144 |
| chr3:187796234 | C | T | rs28464172 | LPP | 6.209576838 | −75452 |
| chr11:100940403 | C | T | rs11600372 | PGR | 4.952841235 | 59391 |
| chr2:64118877 | C | T | rs6546039 | UGP2 | 6.261400133 | 49863 |
| chr5:130253689 | G | C | rs115453900 | HINT1 | 4.97793823 | 247260 |
| chr12:98952797 | G | A | rs12300113 | SLC25A3 | 4.504795009 | −34606 |
| chr6:109701218 | T | C | rs1358998 | CD164 | 4.986165119 | 1724 |
| chr9:89498025 | T | G | rs17052300 | GAS1 | 5.490031987 | 64396 |
| chr4:108934215 | C | A | rs6837149 | HADH | 4.666678868 | 8395 |
| chr10:45105329 | C | T | rs11239170 | CXCL12 | 5.279829651 | −224784 |
| chr2:216307134 | G | A | rs55942719 | FN1 | 5.302667692 | −6341 |
| chr19:16194504 | G | A | rs17708984 | TPM4 | 3.919468038 | 6678 |
| chr10:73608385 | T | C | rs148998541 | PSAP | 3.870699406 | 2623 |
| chr10:79261429 | A | G | rs111613954 | KCNMA1 | 6.443343088 | 136137 |
| chr1:155971372 | A | T | rs141188347 | SSR2 | 4.602028807 | 19370 |

TABLE 2
The enriched biological functions for the genes
identified in spontaneous preterm labor.

| Cluster | Description | Type | FDR |
|---|---|---|---|
| C1 | actin cytoskeleton | CC | 2.51E−27 |
| C1 | supramolecular fiber organization | BP | 1.15E−20 |
| C1 | supramolecular fiber | CC | 2.96E−20 |
| C1 | supramolecular polymer | CC | 4.58E−20 |
| C1 | contractile fiber | CC | 1.83E−19 |
| C1 | myofibril | CC | 7.29E−18 |
| C1 | actin filament-based process | BP | 1.22E−17 |
| C1 | supramolecular complex | CC | 2.12E−17 |
| C1 | actin cytoskeleton organization | BP | 1.38E−16 |
| C1 | sarcomere | CC | 4.22E−14 |
| C1 | cytoskeleton organization | BP | 1.11E−11 |
| C1 | actin filament bundle | CC | 4.16E−11 |
| C1 | cell cortex | CC | 4.78E−11 |
| C1 | actin filament organization | BP | 5.30E−10 |
| C1 | contractile actin filament bundle | CC | 2.07E−09 |
| C1 | stress fiber | CC | 2.07E−09 |
| C1 | actin filament | CC | 2.96E−09 |
| C1 | I band | CC | 5.81E−09 |
| C1 | actomyosin | CC | 1.17E−08 |
| C1 | Z disc | CC | 1.27E−08 |
| C1 | regulation of actin filament-based | BP | 3.07E−08 |
| | process | | |
| C1 | cortical cytoskeleton | CC | 4.57E−08 |
| C1 | regulation of supramolecular fiber | BP | 3.57E−07 |
| | organization | | |
| C1 | regulation of actin cytoskeleton | BP | 3.75E−07 |
| | organization | | |
| C1 | cortical actin cytoskeleton | CC | 1.08E−06 |
| C1 | regulation of actin filament organization | BP | 1.37E−06 |
| C1 | podosome | CC | 6.47E−06 |
| C1 | regulation of cellular component size | BP | 6.84E−06 |
| C1 | regulation of anatomical structure size | BP | 1.27E−05 |
| C1 | sarcolemma | CC | 1.87E−04 |
| C1 | regulation of cellular component biogenesis | BP | 2.78E−04 |
| C1 | filamentous actin | CC | 2.79E−04 |
| C1 | regulation of organelle organization | BP | 3.04E−04 |
| C1 | regulation of cytoskeleton organization | BP | 3.41E−04 |
| C1 | regulation of actin polymerization or | BP | 4.25E−04 |
| | depolymerization | | |
| C1 | regulation of actin filament length | BP | 4.57E−04 |
| C1 | establishment or maintenance of cell | BP | 5.59E−04 |
| | polarity | | |
| C1 | actin polymerization or depolymerization | BP | 5.97E−04 |
| C1 | negative regulation of cellular component | BP | 6.30E−04 |
| | organization | | |
| C1 | actomyosin structure organization | BP | 7.45E−04 |
| C1 | polymeric cytoskeletal fiber | CC | 1.21E−03 |
| C1 | actin filament depolymerization | BP | 2.36E−03 |
| C1 | brush border | CC | 3.93E−03 |
| C1 | fascia adherens | CC | 4.31E−03 |
| C1 | muscle thin filament tropomyosin | CC | 5.38E−03 |
| C1 | positive regulation of supramolecular fiber | BP | 1.04E−02 |
| | organization | | |
| C1 | regulation of actin filament depolymerization | BP | 1.24E−02 |
| C1 | A band | CC | 1.40E−02 |
| C1 | protein depolymerization | BP | 1.43E−02 |
| C1 | actin filament severing | BP | 1.77E−02 |
| C1 | regulation of actin filament polymerization | BP | 2.32E−02 |
| C1 | cluster of actin-based cell projections | CC | 2.73E−02 |
| C1 | negative regulation of actin filament | BP | 4.73E−02 |
| | polymerization | | |
| C1 | actin filament fragmentation | BP | 4.75E−02 |
| C1 | actin filament bundle assembly | BP | 4.95E−02 |
| C1 | muscle contraction | BP | 8.71E−12 |
| C1 | muscle system process | BP | 1.77E−11 |
| C1 | homeostatic process | BP | 1.50E−03 |
| C1 | regulation of cation transmembrane | BP | 2.96E−03 |
| | transport | | |
| C1 | regulation of muscle contraction | BP | 3.33E−03 |
| C1 | regulation of muscle system process | BP | 4.91E−03 |
| C1 | perinuclear region of cytoplasm | CC | 5.23E−03 |
| C1 | smooth muscle contraction | BP | 6.05E−03 |
| C1 | regulation of transmembrane transport | BP | 6.42E−03 |
| C1 | G protein-coupled receptor signaling | BP | 1.26E−02 |
| | pathway involved in heart process | | |
| C1 | regulation of transferase activity | BP | 1.52E−02 |
| C1 | regulation of ion transport | BP | 1.73E−02 |
| C1 | regulation of protein kinase activity | BP | 1.95E−02 |
| C1 | regulation of ion transmembrane transport | BP | 2.39E−02 |
| C1 | actin filament-based movement | BP | 2.59E−02 |
| C1 | regulation of transmembrane transporter | BP | 3.08E−02 |
| | activity | | |
| C1 | regulation of kinase activity | BP | 4.19E−02 |
| C1 | positive regulation of catalytic activity | BP | 4.37E−02 |
| C1 | muscle structure development | BP | 3.24E−09 |
| C1 | muscle tissue development | BP | 4.62E−06 |
| C1 | muscle organ development | BP | 3.32E−05 |
| C1 | striated muscle tissue development | BP | 5.92E−04 |
| C1 | skeletal muscle tissue development | BP | 1.49E−03 |
| C1 | skeletal muscle organ development | BP | 3.33E−03 |
| C1 | muscle cell development | BP | 2.32E−02 |
| C2 | focal adhesion | CC | 3.95E−32 |
| C2 | cell-substrate junction | CC | 9.69E−32 |
| C2 | anchoring junction | CC | 2.71E−25 |
| C2 | biological adhesion | BP | 6.98E−14 |
| C2 | cell adhesion | BP | 1.97E−13 |
| C2 | localization of cell | BP | 8.42E−12 |
| C2 | cell motility | BP | 8.42E−12 |
| C2 | cell leading edge | CC | 4.76E−11 |
| C2 | cell migration | BP | 1.23E−10 |
| C2 | blood vessel development | BP | 1.66E−10 |
| C2 | cell-substrate junction assembly | BP | 2.11E−10 |
| C2 | regulation of cell migration | BP | 3.47E−10 |
| C2 | cell-substrate junction organization | BP | 5.98E−10 |
| C2 | vasculature development | BP | 8.68E−10 |
| C2 | lamellipodium | CC | 9.17E−10 |
| C2 | regulation of cell motility | BP | 1.02E−09 |
| C2 | blood vessel morphogenesis | BP | 1.14E−09 |
| C2 | circulatory system development | BP | 1.23E−09 |
| C2 | regulation of cellular component movement | BP | 1.44E−09 |
| C2 | regulation of locomotion | BP | 4.82E−09 |
| C2 | regulation of cell morphogenesis | BP | 6.06E−08 |
| C2 | angiogenesis | BP | 7.45E−08 |
| C2 | tube morphogenesis | BP | 1.04E−07 |
| C2 | anatomical structure formation involved | BP | 3.55E−07 |
| | in morphogenesis | | |
| C2 | cell-substrate adhesion | BP | 4.50E−07 |
| C2 | focal adhesion assembly | BP | 4.52E−07 |
| C2 | cell junction assembly | BP | 1.30E−06 |
| C2 | cell junction organization | BP | 1.72E−06 |
| C2 | regulation of anatomical structure | BP | 2.21E−06 |
| | morphogenesis | | |
| C2 | regulation of cell-substrate adhesion | BP | 3.64E−06 |
| C2 | regulation of cell adhesion | BP | 5.10E−06 |
| C2 | tube development | BP | 6.39E−06 |
| C2 | developmental growth | BP | 1.68E−05 |
| C2 | negative regulation of cellular component | BP | 2.50E−05 |
| | movement | | |
| C2 | positive regulation of cell migration | BP | 2.67E−05 |
| C2 | regulation of cell-substrate junction assembly | BP | 2.74E−05 |
| C2 | regulation of focal adhesion assembly | BP | 2.74E−05 |
| C2 | cell-matrix adhesion | BP | 5.56E−05 |
| C2 | growth | BP | 5.84E−05 |
| C2 | regulation of cell-substrate junction | BP | 6.13E−05 |
| | organization | | |
| C2 | positive regulation of cell motility | BP | 6.92E−05 |
| C2 | negative regulation of cell motility | BP | 7.27E−05 |
| C2 | positive regulation of cellular component | BP | 1.13E−04 |
| | movement | | |
| C2 | positive regulation of locomotion | BP | 1.22E−04 |
| C2 | cell-cell junction | CC | 2.41E−04 |
| C2 | negative regulation of locomotion | BP | 3.36E−04 |
| C2 | positive regulation of cell adhesion | BP | 3.87E−04 |
| C2 | positive regulation of cell-substrate | BP | 5.14E−04 |
| | adhesion | | |
| C2 | negative regulation of cell migration | BP | 5.94E−04 |
| C2 | regulation of cell shape | BP | 1.18E−03 |
| C2 | cell-cell contact zone | CC | 1.32E−03 |
| C2 | regulation of cell junction assembly | BP | 1.36E−03 |
| C2 | cell-cell adhesion | BP | 1.36E−03 |
| C2 | tissue migration | BP | 1.49E−03 |
| C2 | tissue regeneration | BP | 2.43E−03 |
| C2 | caveola | CC | 2.75E−03 |
| C2 | regulation of cell-matrix adhesion | BP | 4.63E−03 |
| C2 | regeneration | BP | 7.31E−03 |
| C2 | integrin-mediated signaling pathway | BP | 7.92E−03 |
| C2 | epithelium development | BP | 8.30E−03 |
| C2 | adherens junction | CC | 8.40E−03 |
| C2 | intercalated disc | CC | 8.59E−03 |
| C2 | epithelial cell proliferation | BP | 1.00E−02 |
| C2 | plasma membrane region | CC | 1.07E−02 |
| C2 | ameboidal-type cell migration | BP | 1.08E−02 |
| C2 | regulation of hepatocyte proliferation | BP | 1.26E−02 |
| C2 | epithelial cell migration | BP | 1.44E−02 |
| C2 | epithelium migration | BP | 1.62E−02 |
| C2 | gland morphogenesis | BP | 1.68E−02 |
| C2 | gland development | BP | 1.82E−02 |
| C2 | tissue morphogenesis | BP | 3.15E−02 |
| C2 | animal organ morphogenesis | BP | 3.82E−02 |
| C2 | regulation of leukocyte migration | BP | 4.05E−02 |
| C2 | regulation of epithelial cell proliferation | BP | 4.48E−02 |
| C2 | apical junction complex | CC | 4.78E−02 |
| C3 | secretory granule lumen | CC | 1.19E−09 |
| C3 | cytoplasmic vesicle lumen | CC | 1.59E−09 |
| C3 | vesicle lumen | CC | 1.84E−09 |
| C3 | secretory granule | CC | 2.12E−08 |
| C3 | secretory vesicle | CC | 1.80E−07 |
| C3 | regulated exocytosis | BP | 4.20E−07 |
| C3 | response to cytokine | BP | 2.76E−06 |
| C3 | exocytosis | BP | 7.45E−06 |
| C3 | platelet degranulation | BP | 1.02E−05 |
| C3 | cell activation | BP | 4.19E−05 |
| C3 | immune effector process | BP | 7.83E−05 |
| C3 | endoplasmic reticulum | CC | 8.27E−05 |
| C3 | neutrophil mediated immunity | BP | 8.31E−05 |
| C3 | neutrophil degranulation | BP | 1.53E−04 |
| C3 | neutrophil activation involved in immune | BP | 1.89E−04 |
| | response | | |
| C3 | myeloid leukocyte mediated immunity | BP | 2.34E−04 |
| C3 | lumenal side of membrane | CC | 2.79E−04 |
| C3 | cellular response to cytokine stimulus | BP | 2.97E−04 |
| C3 | neutrophil activation | BP | 3.35E−04 |
| C3 | platelet alpha granule lumen | CC | 3.95E−04 |
| C3 | granulocyte activation | BP | 4.42E−04 |
| C3 | MHC protein complex | CC | 6.12E−04 |
| C3 | cell activation involved in immune | BP | 6.90E−04 |
| | response | | |
| C3 | ficolin-1-rich granule | CC | 7.04E−04 |
| C3 | lumenal side of endoplasmic reticulum | CC | 7.94E−04 |
| | membrane | | |
| C3 | integral component of lumenal side of | CC | 7.94E−04 |
| | endoplasmic | | |
| | reticulum membrane | | |
| C3 | leukocyte degranulation | BP | 1.32E−03 |
| C3 | MHC class I protein complex | CC | 1.47E−03 |
| C3 | leukocyte activation involved in immune | BP | 1.84E−03 |
| | response | | |
| C3 | ficolin-1-rich granule lumen | CC | 2.01E−03 |
| C3 | myeloid cell activation involved in immune | BP | 2.17E−03 |
| | response | | |
| C3 | leukocyte mediated immunity | BP | 2.87E−03 |
| C3 | export from cell | BP | 2.95E−03 |
| C3 | myeloid leukocyte activation | BP | 3.40E−03 |
| C3 | leukocyte activation | BP | 3.77E−03 |
| C3 | secretion by cell | BP | 4.62E−03 |
| C3 | platelet alpha granule | CC | 5.34E−03 |
| C3 | secretion | BP | 6.05E−03 |
| C3 | vesicle membrane | CC | 8.42E−03 |
| C3 | antigen processing and presentation of | BP | 9.73E−03 |
| | endogenous antigen | | |
| C3 | cell surface | CC | 1.04E−02 |
| C3 | antigen processing and presentation of | BP | 1.20E−02 |
| | endogenous peptide antigen via MHC | | |
| | class I via ER pathway, | | |
| C3 | antigen processing and presentation of | BP | 1.20E−02 |
| | endogenous peptide antigen via MHC | | |
| | class I via ER pathway, TAP-independent | | |
| C3 | ER to Golgi transport vesicle membrane | CC | 1.23E−02 |
| C3 | antigen processing and presentation of | BP | 1.34E−02 |
| | exogenous peptide antigen via MHC | | |
| | class I, TAP-independent | | |
| C3 | endocytic vesicle | CC | 1.34E−02 |
| C3 | endosome membrane | CC | 1.68E−02 |
| C3 | response to interferon-gamma | BP | 1.86E−02 |
| C3 | viral process | BP | 2.15E−02 |
| C3 | antigen processing and presentation of | BP | 3.23E−02 |
| | endogenous peptide antigen | | |
| C3 | COPII-coated ER to Golgi transport vesicle | CC | 3.65E−02 |
| C3 | vacuolar lumen | CC | 4.61E−02 |
| C4 | regulation of cell death | BP | 1.34E−11 |
| C4 | regulation of apoptotic process | BP | 5.73E−11 |
| C4 | regulation of programmed cell death | BP | 6.81E−11 |
| C4 | apoptotic process | BP | 7.22E−10 |
| C4 | response to abiotic stimulus | BP | 2.03E−08 |
| C4 | negative regulation of cell death | BP | 2.12E−07 |
| C4 | negative regulation of programmed cell | BP | 3.07E−07 |
| | death | | |
| C4 | negative regulation of apoptotic process | BP | 4.47E−07 |
| C4 | apoptotic signaling pathway | BP | 1.31E−04 |
| C4 | positive regulation of cell death | BP | 2.14E−04 |
| C4 | positive regulation of apoptotic process | BP | 4.77E−04 |
| C4 | negative regulation of response to stimulus | BP | 6.48E−04 |
| C4 | regulation of apoptotic signaling pathway | BP | 7.52E−04 |
| C4 | negative regulation of signal transduction | BP | 8.29E−04 |
| C4 | positive regulation of programmed cell death | BP | 8.56E−04 |
| C4 | negative regulation of cell communication | BP | 3.03E−03 |
| C4 | negative regulation of signaling | BP | 3.21E−03 |
| C4 | response to hypoxia | BP | 3.26E−03 |
| C4 | response to decreased oxygen levels | BP | 6.15E−03 |
| C4 | negative regulation of apoptotic signaling | BP | 7.58E−03 |
| | pathway | | |
| C4 | regulation of extrinsic apoptotic signaling | BP | 8.09E−03 |
| | pathway | | |
| C4 | cellular response to hypoxia | BP | 9.78E−03 |
| C4 | biological process involved in symbiotic | BP | 1.00E−02 |
| | interaction | | |
| C4 | cellular response to oxygen levels | BP | 1.05E−02 |
| C4 | positive regulation of multicellular | BP | 1.49E−02 |
| | organismal process | | |
| C4 | positive regulation of cell communication | BP | 1.55E−02 |
| C4 | positive regulation of signaling | BP | 1.69E−02 |
| C4 | cellular response to decreased oxygen levels | BP | 1.71E−02 |
| C4 | response to oxygen levels | BP | 1.84E−02 |
| C4 | positive regulation of developmental process | BP | 2.57E−02 |
| C4 | negative regulation of intracellular signal | BP | 2.58E−02 |
| | transduction | | |
| C4 | aggrephagy | BP | 2.90E−02 |
| C4 | regulation of response to stress | BP | 3.05E−02 |
| C4 | positive regulation of signal transduction | BP | 3.05E−02 |
| C4 | response to unfolded protein | BP | 3.75E−02 |
| C4 | positive regulation of extrinsic apoptotic | BP | 3.79E−02 |
| | signaling pathway | | |
| C4 | response to endogenous stimulus | BP | 2.78E−09 |
| C4 | response to oxygen-containing compound | BP | 3.00E−07 |
| C4 | response to growth factor | BP | 2.38E−06 |
| C4 | cellular response to endogenous stimulus | BP | 6.09E−06 |
| C4 | regulation of cell population proliferation | BP | 9.95E−06 |
| C4 | cellular response to growth factor stimulus | BP | 1.01E−05 |
| C4 | response to organonitrogen compound | BP | 2.43E−05 |
| C4 | response to inorganic substance | BP | 4.87E−05 |
| C4 | membrane microdomain | CC | 6.96E−05 |
| C4 | membrane raft | CC | 6.96E−05 |
| C4 | response to nitrogen compound | BP | 8.75E−05 |
| C4 | response to metal ion | BP | 3.23E−04 |
| C4 | negative regulation of cell population | BP | 7.91E−04 |
| | proliferation | | |
| C4 | response to calcium ion | BP | 7.96E−04 |
| C4 | response to organic cyclic compound | BP | 1.08E−03 |
| C4 | cellular response to transforming growth | BP | 1.49E−03 |
| | factor beta stimulus | | |
| C4 | response to CAMP | BP | 1.69E−03 |
| C4 | response to transforming growth factor | BP | 2.07E−03 |
| | beta | | |
| C4 | response to mechanical stimulus | BP | 2.39E−03 |
| C4 | response to oxidative stress | BP | 2.61E−03 |
| C4 | response to drug | BP | 4.47E−03 |
| C4 | enzyme linked receptor protein signaling | BP | 4.94E−03 |
| | pathway | | |
| C4 | response to ketone | BP | 5.09E−03 |
| C4 | plasma membrane raft | CC | 6.30E−03 |
| C4 | response to hormone | BP | 6.30E−03 |
| C4 | transforming growth factor beta receptor | BP | 8.72E−03 |
| | signaling pathway | | |
| C4 | basal part of cell | CC | 1.25E−02 |
| C4 | cellular response to oxygen-containing | BP | 1.37E−02 |
| | compound | | |
| C4 | response to progesterone | BP | 1.78E−02 |
| C4 | basal plasma membrane | CC | 2.24E−02 |
| C4 | regulation of cellular response to growth | BP | 4.08E−02 |
| | factor stimulus | | |
| C4 | reproductive structure development | BP | 4.20E−02 |
| C4 | reproductive system development | BP | 4.65E−02 |
| C5 | cell morphogenesis | BP | 1.77E−08 |
| C5 | cell projection organization | BP | 1.87E−05 |
| C5 | plasma membrane bounded cell projection | BP | 1.92E−05 |
| | organization | | |
| C5 | positive regulation of cellular component | BP | 4.24E−05 |
| | organization | | |
| C5 | cellular component morphogenesis | BP | 6.89E−05 |
| C5 | cell morphogenesis involved in | BP | 1.70E−04 |
| | differentiation | | |
| C5 | ruffle | CC | 2.86E−03 |
| C5 | regulation of plasma membrane bounded | BP | 4.19E−03 |
| | cell projection organization | | |
| C5 | regulation of cell projection organization | BP | 7.24E−03 |
| C5 | neurogenesis | BP | 1.07E−02 |
| C5 | neuron projection development | BP | 1.09E−02 |
| C5 | neuron development | BP | 1.53E−02 |
| C5 | cell projection morphogenesis | BP | 1.62E−02 |
| C5 | distal axon | CC | 1.64E−02 |
| C5 | growth cone | CC | 1.82E−02 |
| C5 | site of polarized growth | CC | 2.47E−02 |
| C5 | cell part morphogenesis | BP | 2.57E−02 |
| C5 | cell growth | BP | 3.53E−02 |
| C5 | bleb assembly | BP | 3.91E−02 |
| C6 | regulation of cellular localization | BP | 2.57E−08 |
| C6 | regulation of cellular protein localization | BP | 8.32E−08 |
| C6 | positive regulation of cellular protein | BP | 1.35E−06 |
| | localization | | |
| C6 | regulation of protein localization | BP | 9.04E−06 |
| C6 | cellular macromolecule localization | BP | 2.35E−05 |
| C6 | cellular protein localization | BP | 4.28E−05 |
| C6 | positive regulation of protein localization | BP | 1.36E−04 |
| | to membrane | | |
| C6 | regulation of protein localization to | BP | 1.75E−04 |
| | membrane | | |
| C6 | regulation of transport | BP | 5.25E−04 |
| C6 | intracellular transport | BP | 6.87E−04 |
| C6 | protein localization to membrane | BP | 8.51E−04 |
| C6 | localization within membrane | BP | 9.90E−04 |
| C6 | positive regulation of transport | BP | 1.37E−02 |
| C6 | maintenance of protein location | BP | 1.58E−02 |
| C6 | positive regulation of intracellular protein | BP | 3.97E−02 |
| | transport | | |
| C6 | regulation of intracellular transport | BP | 4.03E−02 |
| C6 | maintenance of protein location in cell | BP | 4.24E−02 |
| C7 | negative regulation of molecular function | BP | 2.07E−08 |
| C7 | negative regulation of catalytic activity | BP | 6.38E−08 |
| C7 | negative regulation of protein metabolic | BP | 1.32E−06 |
| | process | | |
| C7 | negative regulation of cellular protein | BP | 1.15E−05 |
| | metabolic process | | |
| C7 | regulation of proteolysis | BP | 1.66E−03 |
| C7 | negative regulation of proteolysis | BP | 2.00E−03 |
| C7 | negative regulation of hydrolase activity | BP | 2.51E−03 |
| C7 | aging | BP | 2.78E−03 |
| C7 | regulation of hydrolase activity | BP | 3.32E−03 |
| C7 | negative regulation of endopeptidase | BP | 3.75E−03 |
| | activity | | |
| C7 | regulation of peptidase activity | BP | 3.82E−03 |
| C7 | negative regulation of peptidase | BP | 7.45E−03 |
| | activity | | |
| C7 | modulation of age-related behavioral | BP | 3.91E−02 |
| | decline | | |
| C7 | regulation of endopeptidase activity | BP | 4.81E−02 |
| C8 | response to wounding | BP | 9.79E−12 |
| C8 | wound healing | BP | 5.79E−10 |
| C8 | positive regulation of gene expression | BP | 8.46E−06 |
| C8 | blood coagulation | BP | 1.59E−03 |
| C8 | hemostasis | BP | 1.91E−03 |
| C8 | coagulation | BP | 2.19E−03 |
| C8 | blood microparticle | CC | 5.96E−03 |
| C8 | platelet aggregation | BP | 3.01E−02 |
| C9 | collagen-containing extracellular matrix | CC | 2.36E−20 |
| C9 | extracellular matrix | CC | 3.24E−16 |
| C9 | external encapsulating structure | CC | 3.47E−16 |
| C9 | extracellular matrix organization | BP | 1.50E−08 |
| C9 | extracellular structure organization | BP | 1.60E−08 |
| C9 | external encapsulating structure | BF | 1.81E−08 |
| | organization | | |
| C9 | endoplasmic reticulum lumen | CC | 2.51E−08 |
| C9 | basement membrane | CC | 1.75E−04 |
| C9 | collagen fibril organization | BP | 8.15E−04 |
| C9 | collagen trimer | CC | 1.14E−03 |
| C9 | collagen beaded filament | CC | 1.36E−03 |
| C9 | collagen type VI trimer | CC | 1.36E−03 |
| C9 | lytic vacuole | CC | 8.05E−03 |
| C9 | lysosome | CC | 8.05E−03 |
| C9 | banded collagen fibril | CC | 9.89E−03 |
| C9 | fibrillar collagen trimer | CC | 9.89E−03 |
| C9 | vacuole | CC | 2.80E−02 |

TABLE 3
The lists of G1 and G2 genes.

| Gene Symbol | Group |
|---|---|
| ACTG1 | G2 |
| AHNAK | G1 |
| ANTXR2 | G1 |
| ATF3 | G2 |
| ATP1B1 | G1 |
| ATP2B4 | G1 |
| CALM2 | G1 |
| CAPZA2 | G1 |
| CAV1 | G1 |
| CDC42EP3 | G1 |
| CFL1 | G2 |
| CHRDL2 | G2 |
| CITED2 | G1 |
| CNN1 | G1 |
| COL18A1 | G2 |
| CORO1C | G1 |
| CPQ | G1 |
| CSDE1 | G1 |
| DCN | G1 |
| DPP6 | G1 |
| DPYSL3 | G1 |
| DST | G1 |
| DSTN | G1 |
| DUSP1 | G2 |
| DYNC1LI2 | G1 |
| EGR1 | G2 |
| ENO1 | G2 |
| FHL1 | G1 |
| FILIP1L | G1 |
| FOS | G2 |
| FOSB | G2 |
| GSN | G1 |
| HADH | G1 |
| HNRNPH1 | G2 |
| HSPA5 | G2 |
| HSPB8 | G1 |
| IER2 | G2 |
| IFITM3 | G2 |
| IGFBP4 | G2 |
| IGFBP7 | G1 |
| ITM2B | G1 |
| JUN | G2 |
| KANK2 | G1 |
| KCNMA1 | G1 |
| KDELR2 | G2 |
| LDB2 | G1 |
| MAP4 | G1 |
| MBNL1 | G1 |
| MCL1 | G2 |
| MFAP5 | G1 |
| MGP | G1 |
| MSRB3 | G1 |
| MYH11 | G1 |
| MYLK | G1 |
| MYO1C | G1 |
| NME2 | G2 |
| NR2F2 | G1 |
| PALLD | G1 |
| PARVA | G1 |
| PBX1 | G1 |
| PGR | G1 |
| PKD2 | G1 |
| PLN | G1 |
| PLS3 | G1 |
| PPIB | G2 |
| PPP1R12B | G1 |
| PRUNE2 | G1 |
| PTMA | G2 |
| PTN | G1 |
| RAP2C | G1 |
| RGS2 | G2 |
| RHOB | G2 |
| RSPO3 | G1 |
| SEC61B | G2 |
| SERINC1 | G1 |
| SH3BGRL | G1 |
| SLMAP | G1 |
| SORBS1 | G1 |
| SPARCL1 | G1 |
| SSR2 | G2 |
| SUN1 | G1 |
| SVIL | G1 |
| SYNPO2 | G1 |
| TACC1 | G1 |
| TBC1D1 | G1 |
| TCEAL4 | G1 |
| TES | G1 |
| TGM2 | G2 |
| THBS1 | G2 |
| TIMP2 | G1 |
| TJP1 | G1 |
| TMEM123 | G1 |
| TNS1 | G1 |
| TPI1 | G2 |
| TPM1 | G1 |
| YAP1 | G1 |
| YWHAZ | G1 |
| ZFP36 | G2 |
| ZFP36L1 | G2 |

TABLE 4
The information for the small molecules tested in the experiments.

| Reagents | CAS # | Lot # | Manufacturers |
|---|---|---|---|
| RKI-1447 | 1342278-01-6 | 50-136-4509 | Selleck |
| | | | Chemical |
| U-0126 | 1173097-76-1 | 50-187-2573 | Medchemexpress |
| 4,5,6,7- | 17374-26-4 | 50-194-8333 | Selleck |
| tetrabromobenzotriazole | | | Chemical |
| LY294002 | 154447-36-6 | 50-187-1783 | Medchemexpress |
| URMC-099 | 1229582-33-5 | 50-187-2702 | Medchemexpress |
| SB203580 | 152121-47-6 | 50-797-4 | Selleck |
| | | | Chemical |
| Bosutinib | 380843-75-4 | 50-193-2174 | Medchemexpress |
| Smooth Muscle Cell | CC-3181 | | Lonza |
| Growth Basal Medium | | | |
| collagen | 5074 | | Sigma-Aldrich |

TABLE 5
The cohort information for individuals with recurrent preterm birth.

| | | | G1 | G2 | HCNDD |
|---|---|---|---|---|---|
| | | Age | High Fi | High Fi | High |
| | | of | Mutation | Mutation | Fi Mutation |
| SampleID | Cohort | Mom | Burden | Burden | Burden |
| S1 | TERM | 27 | 2343 | 314 | 1154 |
| S2 | TERM | 23 | 2352 | 394 | 1171 |
| S3 | PRETERM | 22 | 2517 | 425 | 1242 |
| S4 | PRETERM | 31 | 2324 | 382 | 1240 |
| S5 | TERM | 24 | 2327 | 438 | 1350 |
| S6 | TERM | 19 | 2405 | 395 | 1148 |
| S7 | TERM | 30 | 2278 | 388 | 1305 |
| S8 | TERM | 19 | 2426 | 379 | 1232 |
| S9 | PRETERM | 20 | 2477 | 407 | 1328 |
| S10 | PRETERM | 20 | 2458 | 402 | 1209 |
| S11 | TERM | 21 | 2027 | 262 | 1095 |
| S12 | TERM | 21 | 2326 | 354 | 1100 |
| S13 | TERM | 22 | 2384 | 382 | 1259 |
| S14 | TERM | 38 | 2374 | 341 | 1122 |
| S15 | PRETERM | 25 | 2404 | 428 | 1181 |
| S16 | PRETERM | | 2455 | 414 | 1262 |
| S17 | TERM | 23 | 2322 | 410 | 1272 |
| S18 | PRETERM | 27 | 2497 | 402 | 1268 |
| S19 | PRETERM | 24 | 2360 | 345 | 1287 |
| S20 | TERM | 25 | 2357 | 404 | 1283 |
| S21 | TERM | 24 | 2379 | 416 | 1124 |
| S22 | TERM | 30 | 1428 | 234 | 704 |
| S23 | TERM | 28 | 2248 | 434 | 1261 |
| S24 | PRETERM | 34 | 2408 | 364 | 1207 |
| S26 | TERM | 22 | 2445 | 395 | 1206 |
| S27 | TERM | 31 | 2284 | 352 | 1200 |
| S28 | PRETERM | 30 | 2371 | 364 | 1213 |
| S29 | TERM | 17 | 2298 | 406 | 1144 |
| S30 | PRETERM | 23 | 2521 | 392 | 1293 |
| S31 | PRETERM | 23 | 2487 | 385 | 1191 |
| S32 | PRETERM | 25 | 2465 | 393 | 1242 |
| S33 | PRETERM | 33 | 2336 | 452 | 1256 |
| S34 | TERM | 25 | 2379 | 396 | 1208 |
| S35 | TERM | 29 | 2410 | 335 | 1307 |
| S36 | TERM | 28 | 2367 | 409 | 1278 |
| S37 | TERM | 26 | 2281 | 384 | 1198 |
| S38 | PRETERM | 24 | 2402 | 361 | 1255 |
| S39 | TERM | 25 | 2344 | 340 | 1278 |
| S40 | TERM | 34 | 2352 | 423 | 1220 |
| S41 | PRETERM | 22 | 2360 | 382 | 1202 |
| S42 | TERM | 25 | 2325 | 365 | 1117 |
| S43 | TERM | 29 | 2382 | 357 | 1224 |
| S44 | PRETERM | 22 | 2349 | 384 | 1274 |
| S45 | PRETERM | 32 | 1855 | 306 | 805 |
| S46 | PRETERM | 22 | 2487 | 439 | 1237 |
| S47 | TERM | 37 | 2452 | 401 | 1311 |
| S48 | PRETERM | 27 | 2343 | 409 | 1278 |
| S49 | TERM | 21 | 2299 | 389 | 1205 |

What is claimed is:

1. A method for genome-wide identification of non-coding somatic mutations associated with preterm birth, the method comprising:
a) providing a database comprising epigenomic correlation data for associations between non-coding somatic mutations and chromatin structural changes associated with myometrial transition to preterm labor based on genome-wide epigenomic screening of a population of patients experiencing preterm birth;
b) generating a deep learning model to compute the probability that a given genomic sequence has an open chromatin structure; and
c) using the deep learning model to identify non-coding somatic mutations associated with the myometrial transition to preterm labor, wherein a non-coding somatic mutation is considered to contribute to risk of preterm birth if an allelic change from its corresponding reference wild-type allele to the somatic mutation results in an alteration in predicted chromatin openness based on the deep learning model.

2. The method of claim 1, wherein the deep learning model uses a deep residual neural network or deep convolutional neural network.

3. The method of claim 2, further comprising calculating deep estimation from epigenome prediction plus (DEEP+) scores for each non-coding somatic mutation that is identified as contributing to the risk of preterm birth for an individual.

4. The method of claim 3, wherein the DEEP+ scores are used in combination with genome-wide association study (GWAS) risk scores for each non-coding somatic mutation to determine the risk of preterm birth for an individual.

5. The method of claim 3 or 4, wherein the DEEP+ scores are used in combination with haploinsufficiency scores for each non-coding somatic mutation to determine the risk of preterm birth for an individual.

6. The method of any one of claims 3 to 5, wherein the DEEP+ scores are used in combination with myometrial transcriptomic profiling data to determine the risk of preterm birth for an individual.

7. The method of claim 6, further comprising using a Bayesian estimation for altered regulation (BEAR) model to calculate a BEAR composite risk score for each non-coding somatic mutation that is identified as contributing to risk of preterm birth, wherein the BEAR model uses a mixture Gaussian model of the distribution of the DEEP+ scores, the GWAS risk scores, the gene haploinsufficiency scores, and the myometrial transcriptomic profiling data to calculate the BEAR composite risk score, wherein the BEAR composite risk score is used to determine the risk of preterm birth for an individual.

8. The method of any one of claims 1 to 7, wherein the patient is European or African American.

9. The method of any one of claims 1 to 8, wherein one or more of the non-coding somatic mutations are in genes that regulate myometrial muscle relaxation or inflammatory responses.

10. The method of any one of claims 1 to 9, wherein the non-coding somatic mutations are in an intronic genomic region, a promoter, a 5′ untranslated region (5′ UTR), a 3′ untranslated region (3′ UTR), an exonic genomic region, an intergenic genomic region, or a genomic region encoding a non-coding RNA.

11. The method of any one of claims 1 to 10, wherein the non-coding somatic mutations comprise at least one insertion, deletion, or single-nucleotide variant.

12. The method of any one of claims 1 to 11, wherein the epigenomic correlation data comprises assay for transposase-accessible chromatin sequencing (ATAC-Seq) data.

13. A method of predicting risk of preterm birth for an individual, the method comprising:
a) obtaining a biological sample from the individual;
b) genotyping one or more cells in the biological sample to determine if the individual has one or more non-coding somatic mutations associated with risk of preterm birth; and
c) calculating a composite DEEP+ score for the one or more non-coding somatic mutations associated with risk of preterm birth detected by genotyping, wherein the composite DEEP+ score indicates the risk of preterm birth.

14. The method of claim 13, wherein the composite DEEP+ score is used in combination with genome-wide association study (GWAS) risk scores for each non-coding somatic mutation associated with risk of preterm birth detected by genotyping to determine the risk of preterm birth.

15. The method of claim 13 or 14, wherein the composite DEEP+ score is used in combination with haploinsufficiency scores for each non-coding somatic mutation associated with risk of preterm birth detected by genotyping to determine the risk of preterm birth.

16. The method of any one of claims 13 to 15, wherein the composite DEEP+ score is used in combination with myometrial transcriptomic profiling data to determine the risk of preterm birth.

17. The method of claim 16, further comprising using a Bayesian estimation for altered regulation (BEAR) model to calculate a composite BEAR risk score for each non-coding somatic mutation associated with risk of preterm birth detected by genotyping, wherein the BEAR model uses a mixture Gaussian model of the distribution of the DEEP+ scores, the GWAS risk scores, the gene haploinsufficiency scores, and the myometrial transcriptomic profiling data to calculate the composite BEAR risk score, wherein the composite BEAR risk score indicates the risk of preterm birth.

18. The method of any one of claims 13 to 17, wherein the non-coding somatic mutations are in an intronic genomic region, a promoter, a 5′ untranslated region (5′ UTR), a 3′ untranslated region (3′ UTR), an exonic genomic region, an intergenic genomic region, or a genomic region encoding a non-coding RNA.

19. The method of any one of claims 13 to 18, wherein the non-coding somatic mutations comprise at least one insertion, deletion, or single-nucleotide variant.

20. The method of any one of claims 13 to 19, wherein the one or more non-coding somatic mutations associated with risk of preterm birth comprise one or more non-coding somatic mutations selected from Table 1.

21. The method of any one of claims 13 to 20, wherein said genotyping comprises sequencing at least part of a genome of a cell from the biological sample.

22. The method of claim 21, wherein said genotyping comprises sequencing the whole genome of a cell from the biological sample.

23. The method of any one of claims 13 to 22, wherein the biological sample is a myometrium sample.

24. The method of any one of claims 13 to 23, further comprising treating the individual to reduce risk of preterm birth if the composite DEEP+ score indicates the individual is at risk of preterm birth.

25. The method of any one of claims 13 to 23, further comprising treating the individual to reduce risk of preterm birth if the composite BEAR risk score indicates the individual is at risk of preterm birth.

26. The method of claim 24 or 25, wherein said treating comprises administering progestin to the individual.

27. The method of any one of claims 13 to 26, further comprising predicting non-responsiveness of the individual to treatment with progestin based on identifying one or more non-coding somatic mutations in one or more genes selected from the group consisting of AHNAK, ANTXR2, ATP1B1, ATP2B4, CALM2, CAPZA2, CAV1, CDC42EP3, CITED2, CNN1, CORO1C, CPQ, CSDE1, DCN, DPP6, DPYSL3, DST, DSTN, DYNC1LI2, FHL1, FILIP1L, GSN, HADH, HSPB8, IGFBP7, ITM2B, KANK2, KCNMA1, LDB2, MAP4, MBNL1, MFAP5, MGP, MSRB3, MYH11, MYLK, MYO1C, NR2F2, PALLD, PARVA, PBX1, PGR, PKD2, PLN, PLS3, PPP1R12B, PRUNE2, PTN, RAP2C, RSPO3, SERINC1, SH3BGRL, SLMAP, SORBS1, SPARCL1, SUN1, SVIL, SYNPO2, TACC1, TBC1D1, TCEAL4, TES, TIMP2, TJP1, TMEM123, TNS1, TPM1, YAP1, and YWHAZ.

28. A database comprising deep estimation from epigenome prediction plus (DEEP+) scores for a plurality of non-coding somatic mutations associated with preterm birth.

29. The database of claim 28, wherein the database comprises or consists of DEEP+ scores for non-coding somatic mutations selected from Table 1.

30. The database of claim 28 or 29, wherein the database further comprises Bayesian estimation for altered regulation (BEAR) risk scores for the plurality of non-coding somatic mutations associated with preterm birth.

31. A computer implemented method for predicting risk of preterm birth for an individual, the computer performing steps comprising:
a) receiving genome sequencing data for an individual;
b) identifying non-coding somatic mutations associated with preterm birth present in the individual from the genome sequencing data, wherein the individual has a plurality of non-coding somatic mutations selected from Table 1;
c) calculating a composite deep estimation from epigenome prediction plus (DEEP+) score for the non-coding somatic mutations detected in the individual by genotyping using the database of any one of claims 28 to 30, wherein the composite DEEP+ score indicates the risk of preterm birth for the individual; and
d) displaying information regarding the risk of preterm birth for the individual.

32. The computer implemented method of claim 31, further comprising calculating a composite Bayesian estimation for altered regulation (BEAR) risk score for the non-coding somatic mutations detected in the individual by genotyping using the database, wherein the composite BEAR risk score indicates the risk of preterm birth for the individual.

33. The computer implemented method of claim 31 or 32, further comprising storing the information regarding the risk of preterm birth for the individual in a database.

34. A system for predicting the risk of preterm birth for an individual using the computer implemented method of any one of claims 31 to 33, the system comprising:
a) a storage component for storing data, wherein the storage component has instructions for predicting the risk of preterm birth for an individual based on analysis of the genome sequencing data stored therein;
b) a computer processor for processing the genome sequencing data using one or more algorithms, wherein the computer processor is coupled to the storage component and configured to execute the instructions stored in the storage component in order to receive the inputted genome sequencing data and analyze the data according to the computer implemented method of any one of claims 31 to 33; and
c) a display component for displaying the information regarding the risk of preterm birth for the individual.

35. A non-transitory computer-readable medium comprising program instructions that, when executed by a processor in a computer, causes the processor to perform the computer implemented method of any one of claims 31 to 33.

36. A kit comprising the non-transitory computer-readable medium of claim 35 and instructions for predicting the risk of preterm birth for an individual.

37. A method of predicting therapeutic responsiveness of an individual to treatment with progestin for preterm labor, the method comprising:
a) obtaining a biological sample from the individual;
b) genotyping one or more cells in the biological sample to determine if the individual has one or more non-coding somatic mutations associated with risk of preterm birth in one or more genes selected from the group consisting of AHNAK, ANTXR2, ATP1B1, ATP2B4, CALM2, CAPZA2, CAV1, CDC42EP3, CITED2, CNN1, CORO1C, CPQ, CSDE1, DCN, DPP6, DPYSL3, DST, DSTN, DYNC1LI2, FHL1, FILIP1L, GSN, HADH, HSPB8, IGFBP7, ITM2B, KANK2, KCNMA1, LDB2, MAP4, MBNL1, MFAP5, MGP, MSRB3, MYH11, MYLK, MYO1C, NR2F2, PALLD, PARVA, PBX1, PGR, PKD2, PLN, PLS3, PPP1R12B, PRUNE2, PTN, RAP2C, RSPO3, SERINC1, SH3BGRL, SLMAP, SORBS1, SPARCL1, SUN1, SVIL, SYNPO2, TACC1, TBC1D1, TCEAL4, TES, TIMP2, TJP1, TMEM123, TNS1, TPM1, YAP1, and YWHAZ; and
c) calculating a composite DEEP+ score for the one or more non-coding somatic mutations detected in the individual by genotyping using the database of any one of claims 28 to 30, wherein if the composite DEEP+ score is above a reference threshold value, the individual is identified as a non-responder who will not benefit from the treatment with the progestin, and wherein if the composite DEEP+ score is below a reference threshold value, the individual is identified as a responder who will benefit from the treatment with the progestin.

38. The method of claim 37, further comprising calculating a composite Bayesian estimation for altered regulation (BEAR) risk score for the non-coding somatic mutations detected in the individual by genotyping using the database, wherein if the composite BEAR risk score is above a reference threshold value, the individual is identified as a non-responder who will not benefit from the treatment with the progestin, and wherein if the composite BEAR risk score is below a reference threshold value, the individual is identified as a responder who will benefit from the treatment with the progestin.

39. The method of claim 37 or 38, further comprising administering progestin to the individual if the individual is identified as a responder.

40. The method of any one of claims 37 to 39, wherein said genotyping comprises sequencing at least part of a genome of a cell from the biological sample.

41. The method of claim 40, wherein said genotyping comprises sequencing the whole genome of a cell from the biological sample.

42. The method of any one of claims 37 to 41, wherein the biological sample is a myometrium sample.

43. A computer implemented method for predicting therapeutic responsiveness of an individual to treatment with progestin for preterm labor, the computer performing steps comprising:
a) receiving genome sequencing data for an individual;
b) identifying one or more non-coding somatic mutations associated with risk of preterm birth in one or more genes selected from the group consisting of AHNAK, ANTXR2, ATP1B1, ATP2B4, CALM2, CAPZA2, CAV1, CDC42EP3, CITED2, CNN1, CORO1C, CPQ, CSDE1, DCN, DPP6, DPYSL3, DST, DSTN, DYNC1LI2, FHL1, FILIP1L, GSN, HADH, HSPB8, IGFBP7, ITM2B, KANK2, KCNMA1, LDB2, MAP4, MBNL1, MFAP5, MGP, MSRB3, MYH11, MYLK, MYO1C, NR2F2, PALLD, PARVA, PBX1, PGR, PKD2, PLN, PLS3, PPP1R12B, PRUNE2, PTN, RAP2C, RSPO3, SERINC1, SH3BGRL, SLMAP, SORBS1, SPARCL1, SUN1, SVIL, SYNPO2, TACC1, TBC1D1, TCEAL4, TES, TIMP2, TJP1, TMEM123, TNS1, TPM1, YAP1, and YWHAZ;
c) calculating a composite DEEP+ score for the one or more non-coding somatic mutations detected in the individual by genotyping using the database of any one of claims 28 to 30, wherein if the composite DEEP+ score is above a reference threshold value, the individual is identified as a non-responder who will not benefit from the treatment with the progestin, and wherein if the composite DEEP+ score is below a reference threshold value, the individual is identified as a responder who will benefit from the treatment with the progestin; and
d) displaying information regarding whether the individual is identified as a non-responder or a responder.

44. The computer implemented method of claim 43, further comprising calculating a composite Bayesian estimation for altered regulation (BEAR) risk score for the non-coding somatic mutations detected in the individual by genotyping using the database, wherein if the composite BEAR risk score is above a reference threshold value, the individual is identified as a non-responder who will not benefit from the treatment with the progestin, and wherein if the composite BEAR risk score is below a reference threshold value, the individual is identified as a responder who will benefit from the treatment with the progestin.

45. The computer implemented method of claim 43 or 44, further comprising storing the information regarding whether the individual is identified as a non-responder or a responder in a database.

46. A system for predicting therapeutic responsiveness of an individual to treatment with progestin for preterm labor using the computer implemented method of any one of claims 43 to 45, the system comprising:
a) a storage component for storing data, wherein the storage component has instructions for predicting the therapeutic responsiveness of an individual to treatment with progestin based on analysis of the genome sequencing data stored therein;
b) a computer processor for processing the genome sequencing data using one or more algorithms, wherein the computer processor is coupled to the storage component and configured to execute the instructions stored in the storage component in order to receive the inputted genome sequencing data and analyze the data according to the computer implemented method of any one of claims 43 to 45; and
c) a display component for displaying the information regarding whether the individual is identified as a responder or a non-responder.

47. A non-transitory computer-readable medium comprising program instructions that, when executed by a processor in a computer, causes the processor to perform the computer implemented method of any one of claims 43 to 45.

48. A kit comprising the non-transitory computer-readable medium of claim 47 and instructions for predicting the therapeutic responsiveness of an individual to treatment with progestin.

49. A method of treating preterm labor in a pregnant female subject, the method comprising administering a therapeutically effective amount of a composition comprising RKI-1447, bisindolylmaleimide-ix, TBBt, LY294002, URMC099, Bosutinib, or SB-203580 to the pregnant female subject.

50. The method of claim 49, wherein the composition is administered orally, intravenously, intramuscularly, or vaginally.

51. The method of claim 49, wherein the composition is administered locally to the myometrium.

52. The method of any one of claims 49 to 51, wherein the pregnant female subject is having preterm labor or identified as having a risk of preterm labor.

53. The method of any one of claims 49 to 52, wherein multiple cycles of treatment are administered to the pregnant female subject.

54. The method of claim 53, wherein the composition is administered daily or intermittently.

55. The method of claim 53 or 54, wherein the composition is administered to the pregnant female subject during pregnancy beginning at 16 to 20 weeks of gestation.

56. The method of any one of claims 53 to 55, wherein the composition is administered to the pregnant female subject until delivery.

57. A composition comprising RKI-1447, bisindolylmaleimide-ix, TBBt, LY294002, URMC099, Bosutinib, or SB-203580 for use in a method of treating preterm labor.

58. The composition of claim 57, further comprising a pharmaceutically acceptable excipient.
